(19) United States
(12) Patent Application Publication
(10) Pub. No.: US 2007/0026520 A1
(43) Pub. Date: Feb. 1, 2007

(54) Novel cells, compositions, and methods

(76) Inventor: James H. Kelly, Houston, TX (US)

Correspondence Address: NEEDLE & ROSENBERG, P.C., SUITE 1000, 999 PEACHTREE STREET, ATLANTA, GA, 30309-3915, US

(21) Appl. No.: 11/496,179

(22) Filed: Jul. 31, 2006

Related U.S. Application Data
(63) Continuation-in-part of application No. 11/194,143, filed on Jul. 29, 2005.
(60) Provisional application No. 60/763,823, filed on Jan. 30, 2006.
(60) Provisional application No. 60/764,485, filed on Feb. 1, 2006.
(60) Provisional application No. 60/787,203, filed on Mar. 29, 2006.
(60) Provisional application No. 60/786,865, filed on Mar. 29, 2006.

(51) Int. Cl. C12N 5/08

(52) U.S. Cl. 435/366

(57) ABSTRACT

Disclosed are compositions and methods for producing cells and stem cells.

### I. CROSS-REFERENCE TO RELATED APPLICATIONS

This application is a continuation-in-part of U.S. patent application Ser. No. 11/194,143, filed Jul. 29, 2005. This application also claims the benefit of Application No. 60/786,865, filed Mar. 29, 2006, Application No. 60/787,203, filed Mar. 29, 2006, Application No. 60/764,485, filed Feb. 1, 2006, and Application No. 60/763,823, filed Jan. 30, 2006. application Ser. No. 11/194,143, filed Jul. 29, 2005, Application No. 60/786,865, filed Mar. 29, 2006, Application No. 60/787,203, filed Mar. 29, 2006, Application No. 60/764,485, filed Feb. 1, 2006, and Application No. 60/763,823, filed Jan. 30, 2006 are hereby incorporated herein by reference in their entirety.

### II. BACKGROUND

Pluripotent stem cells, such as human pluripotent stem cells, promise to dramatically alter and extend our ability to both understand and treat many of the chronic illnesses that define modern medicine. From drug discovery, to the generation of monoclonal antibodies, to the production of cell therapies, much of human cell biology expects to be transformed by the ability to generate specific cell types, such as human cell types at will. The medical and industrial application of pluripotent stem cells requires the ability to generate large numbers of a single cell type in vitro. The production of monoclonal antibodies through in vitro immune systems, the production of islets for diabetes treatment, and the production of neural precursors for neural related dysfunction are just a few of the human disease areas needing a steady reliable production of specific cell types. The economic significance of this project is dramatic. The monoclonal antibody application alone is a multibillion dollar industry. The National Institutes of Health estimates that the annual cost of diabetes to the United States is $132 billion (http://diabetes.niddk.nih.gov/dm/pubs/statistics/index.htm# 14). Estimates for the annual national cost of neurodegenerative disease is over $ 100 billion (http://www.alzheimers.org/pubs/prog00.htm#The%20Impact%20of%Alzheimer%92s%20Disease).

Current methods of culturing undifferentiated pluripotential stem cells require the use of a feeder cell layer. Moreover, systems that employ feeder cells (or conditioned media from feeder cell cultures) often use cells from a different species than that of the stem cells being cultivated. For instance, growth-arrested mouse embryonic fibroblasts (MEF) have traditionally been used as the feeder layer to maintain a long-term undifferentiated growth of human embryonic stem cells. Though there has been a report of a feeder-free system for cultivating human embryonic stem cells, it requires the use of conditioned medium from MEF cultures in order to maintain the stem cells in an undifferentiated state. The requirement for components such as serum, feeder cells, and/or conditioned medium complicates the process of cultivating pluripotential stem cells. Moreover, the use of cells, especially xenogeneic cells (or cell products), increases the risk that the resulting pluripotential stem cell populations produced by such methods may be contaminated with unwanted components (e.g., aberrant cells, viruses, cells that may induce an immune response in a recipient of the stem cell population, heterogeneous fusion cells, etc.), thereby compromising, for example, the therapeutic potential of human embryonic stem cells cultured by such methods. To attempt to address the limitations imposed by using xenogeneic feeder cells or conditioned medium from xeno cultures, techniques have recently been developed for culturing human embryonic stem cells that use feeder cell layers made from human fetal and adult fibroblasts, human foreskin fibroblasts, and human adult marrow stromal cells. However, like other conventional human embryonic stem cell culturing techniques, those that use human feeder cells still suffer from the drawback of exposing the undifferentiated cells to undefined culture conditions, serum, and/or conditioned medium. As such, the conditions cannot be optimized, and unwanted differentiation-inducing, pathogenic, or toxic factors may be present.

Disclosed herein are compositions and methods for culturing pluripotent stem cells directly on a solid substrate, such as plastic, without the need for a feeder layer. The availability of these cells can enable the realization of many of the potential applications currently envisioned for human stem cells. Also disclosed herein are novel pluripotent and other cell compositions as well as methods for generating more differentiated cells from pluripotent stem cells in vitro, as well as compositions used in the methods or derived from the methods. The cells that are generated can be cloned, characterized, frozen, and used in any quantity necessary while, for example, maintaining the advantages of a normal karyotype. The availability of these cells can enable the realization of many of the potential applications currently envisioned for human stem cells.

### III. SUMMARY

Disclosed are methods and compositions related to production of cells and cell lines.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the Hay1D human PC cell line. This cell line was cloned from a single Hay1 EG cell. The cells are stained for alkaline phosphatase (AP).

FIG. 2 shows the PC1 cell line as viewed by phase microscopy.

FIG. 3 shows the PC9 cell line as viewed by phase microscopy.

FIG. 4 shows the PC10 cell line as viewed by phase microscopy.

FIG. 5 shows the Hay1D cell line as viewed by phase microscopy.

FIG. 6 shows PCs exhibit standard markers for pluripotent cells. SSEA-1 is a lower magnification than the others to demonstrate that the entire population displays the markers. PCs are uniformly negative for SSEA-4.

FIG. 7 shows massive proliferation of PCs between the day 1 and day 5 after explant.

FIG. 8 shows PCs stain positively for alkaline phosphatase.

FIG. 9 shows PCs express Oct4 and Nanog mRNA. Both Oct4 and Nanog were measured using gene specific primers by quantitative RT-PCR.

FIG. 10 shows oncostatin M supports growth of PCs on plastic, but LIF does not. Hay1 cells were plated in multiwell plates and growth was monitored over the course of 12 days in the presence of either 10 ng/ml oncostatin M plus 25 ng/ml FGF-2 or 10 ng/ml human leukemia inhibitory factor (LIF) plus 25 ng/ml FGF-2. Medium was replaced at two day intervals.

FIG. 11 shows human PCs express high levels of the oncostatin M receptor but very low levels of LIF receptor. FIGS. 11A,B shows PC1 cells in phase contrast (panel A) and the same field examining immunofluorescence for the human LIF receptor (panel B). FIGS. 11C,D shows PC1 cells in phase contrast (panel C) and the same field examining immunofluorescence for the human oncostatin M receptor (panel D).

FIG. 12 shows PCs arrest in the absence of oncostatin M. PC1 cells were plated in multiwell dishes in medium with no growth factors (no factors), in medium with all four of the factors (Control=10 ng/ml Oncostatin M, 10 ng/ml SCF, 25 ng/ml FGF-2, 10 μM forskolin), minus forskolin, minus FGF-2, minus Oncostatin or minus SCF. Removal of oncostatin M alone was the same as adding no factors whatsoever.

FIG. 13 shows FGF-2 induces Oct4 in Hay1 cells. Hay1 cells were cultured in the presence of increasing concentrations of FGF-2 for seven days. Cells were lysed and assayed for Oct4 mRNA using QRTPCR.

FIG. 14 shows Zeocin kills Hay1. Hay1 cells were incubated with the indicated concentration of Zeocin. Then cell number was assayed using an MTT based cell proliferation assay.

FIG. 15 shows Oct4 and Nanog expression in PC cultures maintained in the presence of FGF-2 after oncostatin M and SCF were removed.

FIG. 16 shows Nurr1 and tyrosine hydroxylase expression by RT-PCR in the cells of FIG. 15 after being cultured in FGF-2 plus retinoic acid.

FIG. 17 shows alpha-actinin immunolabeling of PC culture maintained in the presence of FGF-2, forskolin and bromo-cyclic AMP after oncostatin M and SCF were removed.

FIG. 18 shows efficient introduction of plasmids into PCs using nucloeporation. The figure shows cells 24 hours after introduction of a CMV promoted GFP plasmid.

FIG. 19 shows differentiating PCs.

FIG. 20 shows embryoid like bodies formed from PCs.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Numerous authors have written about the possible applications of human pluripotent stem cells (for example, Gearhart, J (1998) Science 282, 1061-1062; Pera, M F, et al., (2000) J. Cell Sci. 113, 5-10; Trounson, A (2001) Reprod Fertil Dev. 2001; 13(7-8):523-32; Sussman, N L, Kelly, J H. (1994) U.S. Pat. No. 5,368,555). These range from target evaluation and toxicity testing in drug discovery to attempting to cure type I diabetes by implanting new beta cells into the pancreas. Each of these applications requires large quantities of typically differentiated cells from a controlled and renewable source.

Stem cells, such as multipotent stem cells (e.g., adult stem cells, MAPCs, MSCs, and HSCs), pluripotential stem cells (e.g., ES cells, EG cells, PC cells, and EC cells), and OISCs can be isolated directly on a solid substrate, such as plastic or glass or the like, and can be maintained without the need of a feeder layer. Other advantages for these cells and the methods of derivation and maintenance and the uses thereof are disclosed herein.

Also disclosed are pluripotent stem cells that are dependent on oncostatin M for derivation and/or maintenance but are LIF-independent.

Also disclosed are compositions and methods for the derivation of a nestin-positive stem cell. The nestin-positive stem cell can be an oncostatin-independent stem cell (OISC). As disclosed herein, nestin-positive stem cells and/or OISCs can be maintained without the need of expensive factors such as oncostatin M and stem cell factor (SCF). Other advantages for these cells and the methods of derivation and maintenance and the uses thereof are disclosed herein.

Also disclosed are compositions and methods for directing the differentiation of stem cells, such as multipotent stem cells (e.g., adult stem cells, MAPCs, MSCs, and HSCs), pluripotential stem cells (e.g., ES cells, EG cells, PC cells, and EC cells), and OISCs. Thus, also disclosed are compositions and methods for producing homogenous or substantially homogenous populations of a desired cell type in vitro. Substantially homogenous means at least 80% homogenous. For example, provided herein are compositions and methods for producing a homogenous or substantially homogenous population of neural progenitor cells (NPCs). Also provided are compositions and methods for producing a homogenous or substantially homogenous population of motor neurons (TUJ1 positive). As another example, provided herein are compositions and methods for producing a homogenous or substantially homogenous population of muscle progenitor cells (myoblasts). Also provided are compositions and methods for producing a homogenous or substantially homogenous population of smooth muscle cells (α-actinin positive).

Also disclosed are cells and/or cell types produced by the disclosed methods. Thus, disclosed is homogenous population of tissue-specific progenitors derived from pluripotent stem cells. For example, provided herein is a homogenous population of neural progenitor cells (NPCs) produced using the compositions and methods provided herein. Thus, also provided is a homogenous population of neurons, astrocytes, motor neurons and/or oligodendrocytes produced using the compositions and methods provided herein. As another example, provided herein is a homogenous population of muscle progenitor cells (myoblasts) produced using the compositions and methods provided herein. Also provided is a homogenous population of skeletal, cardiac, and/or smooth muscle cells produced using the compositions and methods provided herein.

### A. COMPOSITIONS AND METHODS

1. Stem Cells

The herein provided compositions and methods involve the production, maintenance and directed differentiation of stem cells. Stem cells are defined (Gilbert, (1994) DEVELOPMENTAL BIOLOGY, 4th Ed. Sinauer Associates, Inc. Sunderland, M A., p. 354) as cells that are “capable of extensive proliferation, creating more stem cells (self-renewal) as well as more differentiated cellular progeny.” These characteristics can be referred to as stem cell capabilities. Pluripotential stem cells, adult stem cells, blastocyst-derived stem cells, gonadal ridge-derived stem cells, teratoma-derived stem cells, totipotent stem cells, multipotent stem cells, oncostatin-independent stem cell (OISCs), embryonic stem cells (ES), embryonic germ cells (EG), PC cells, and embryonic carcinoma cells (EC) are all examples of stem cells.

Stem cells can have a variety of different properties and categories of these properties. For example in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express Oct 4, Sox2, and Nanog (Rodda et al., J. Biol. Chem. 280, 24731-24737 (2005); Chambers et al., Cell 113, 643-655 (2003)). It is understood that some stem cells will express these at the mRNA level, and still others will also express them at the protein level, on for example, the cell surface or within the cell.

It is understood that stem cells can have any combination of any stem cell property or category or categories and properties discussed herein. For example, some stem cells can express alkaline phosphatase, not express SSEA-1 or in certain embodiments not express SSEA-4, proliferate for at least 20 passages, and be capable of differentiating into any cell type. Another set of stem cells, for example, can express SSEA-1 on the cell surface, and be capable of forming endoderm, mesoderm, and ectoderm tissue and be cultured for over a year without differentiation. Another set of stem cells, for example, could be pluripotent stem cells that express SSEA-1. Another set of stem cells, for example, could be blastocyst-derived stem cells that express alkaline phosphatase.

Stem cells can be cultured using any culture means which promotes the properties of the desired type of stem cell. For example, stem cells can be cultured in the presence of fibroblast growth factor (FGF), leukemia inhibitory factor (LIF), membrane associated steel factor (stem cell factor), and soluble steel factor which will produce pluripotential embryonic stem cells. See U.S. Pat. Nos. 5,690,926; 5,670,372, and 5,453,357, which are all incorporated herein by reference for material at least related to deriving and maintaining pluripotential embryonic stem cells in culture. Stem cells can also be cultured on feeder cells, e.g. embryonic fibroblasts, and dissociated cells can be re-plated on embryonic feeder cells. See for example, U.S. Pat. Nos. 6,200,806 and 5,843,780 which are herein incorporated by reference at least for material related to deriving and maintaining stem cells. Stem cells can also be cultured on a solid substrate, e.g. plastic, glass or the like, absent a feeder layer and/or conditioned media.

One category of stem cells is a pluripotent embryonic stem cell. A “pluripotent stem cell” as used herein means a cell which can give rise to many differentiated cell types in an embryo or adult, including the germ cells (sperm and eggs). Pluripotent stem cells are also capable of self-renewal. Thus, these cells not only populate the germ line and give rise to a plurality of terminally differentiated cells which comprise the adult specialized organs, but also are able to regenerate themselves.

One category of stem cells are cells which are capable of self renewal and which can differentiate into cell types of the mesoderm, ectoderm, and endoderm, but which do not give rise to germ cells, sperm or egg.

Another category of stem cells are stem cells which are capable of self renewal and which can differentiate into cell types of the mesoderm, ectoderm, and endoderm, but which do not give rise to placenta cells.

Another category of stem cells is an adult stem cell which is any type of stem cell that is not derived from an embryo/fetus. For example, recent studies have indicated the presence of a more primitive cell population in the bone marrow capable of self-renewal as well as differentiation into a number of different tissue types other than blood cells. These multi-potential cells were discovered as a minor component in the CD34-plastic-adherent cell population of adult bone marrow, and are variously referred to as mesenchymal stem cells (MSC) (Pittenger, et al., Science 284:143-147 (1999)) or multi-potent adult progenitor cells (MAPC) cells (Furcht, L. T., et al., U.S. patent publication 20040107453 A1). MSC cells do not have a single specific identifying marker, but have been shown to be positive for a number of markers, including CD29, CD90, CD105, and CD73, and negative for other markers, including CD14, CD3, and CD34. Various groups have reported to differentiate MSC cells into myocytes, neurons, pancreatic beta-cells, liver cells, bone cells, and connective tissue. Another group (Wernet et al., U.S. patent publication 20020164794 A1) has described an unrestricted somatic stem cell (USSC) with multi-potential capacity that is derived from a CD45/CD34 population within cord blood. Typically, these stem cells have a limited capacity to generate new cell types and are committed to a particular lineage, although adult stem cells capable of generating all three cell types have been described (for example, United States Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are often referred to as “pluripotent hematopoietic stem cell” for its pluripotency within the hematopoietic lineage. A pluripotent adult stem cell is an adult stem cell having pluripotential capabilities (See for example, United States Patent Publication no. 20040107453, which is U.S. patent application Ser. No. 10/467963).

Another category of stem cells is a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Another category of stem cells is a fetal gonadal derived stem cell which is a pluripotent stem cell which was derived from a cell which was obtained from, for example, a human embryo or fetus at or after the 6, 7, 8, 9, or 10 week, post ovulation, developmental stage. Alkaline phosphatase staining occurs at the 5-6 week stage. Fetal gonadal derived stem cell can be derived, for example, from the gonadal ridge of, for example, a 6-10 week human embryo or fetus.

Another category of stem cells are embryo derived stem cells which are derived from embryos of 150 cells or more up to 6 weeks of gestation. Typically embryo derived stem cells will be derived from cells that arose from the inner cell mass cells of the blastocyst or cells which will be come gonadal ridge cells, which can arise from the inner cell mass cells, such as cells which migrate to the gonadal ridge during development. Another category of stem cells are Morula derived stem cells which are stem cells derived from a Morula stage embryo. Other sets of stem cells are embryonic stem cells, (ES cells), embryonic germ cells (EG cells), PCs, and embryonic carcinoma cells (EC cells).

Also disclosed is another category of stem cells called teratoma-derived stem cells which are stem cells which was derived from a teratocarcinoma and can be characterized by the lack of a normal karyotype. Teratocarcinomas are unusual tumors that, unlike most tumors, are comprised of a wide variety of different tissue types. Studies of teratocarcinoma suggested that they arose from primitive gonadal tissue that had escaped the usual control mechanisms. Such properties and manipulations are generally applicable to teratoma-derived stem cells.

Stem cells can also be classified by their potential for development. One category of stem cells are stem cells that can grow into an entire organism. Another category of stem cells are stem cells (which have pluripotent capabilities as defined above) that cannot grow into a whole organism, but can become any other type of cell in the body. Another category of stem cells are stem cells that can only become particular types of cells: e.g. blood cells, or bone cells. Other categories of stem cells include totipotent, pluripotent, and multipotent stem cells.

Provided herein are compositions and methods for the derivation of a pluripotential stem cell (e.g., ES cell, EG cell, PC cell, and EC cell). As disclosed herein, pluripotential stem cells can be alkaline phosphatase (AP) positive, SSEA-1 positive, and SSEA-4 negative. Pluripotential stem cells can also be nanog positive, Sox2 positive, and Oct-4 positive. Pluripotential stem cells can also be Tcl1 positive, and Tbx3 positive. Pluripotential stem cells can also be Cripto positive, Stellar positive and Daz1 positive. Pluripotential stem cells can express cell surface antigens that bind with antibodies having the binding specificity of monoclonal antibodies TRA-1-60 (ATCC HB-4783) and TRA-1-81 (ATCC HB-4784). Pluripotential stem cells are capable of differentiating into derivatives of endodermal, mesodermal, and ectodermal cells throughout the culture. Further, as disclosed herein, these properties of Pluripotential stem cells can be maintained without a feeder layer for at least 10, 11, 12, 13, 14, 15, 16, 17, 18, 19, 20 passages or for over a year. Pluripotential stem cells can be human or other animal. For example, Pluripotential stem cells can be mouse, guinea pig, rat. cattle, horses, pigs, sheep, goats, etc. Pluripotential stem cells can also be from non-human primates.

Also as provided herein, pluripotential stem cells can be differentiated into multipotent cells (e.g., progenitors) or into more terminally differentiated cells such as heart, liver, neural, pancreatic islet, or virtually any cell of the body.

Pluripotential stem cells can be isolated from fetal material, for example, from gonadal tissues, genital ridges, mesenteries or embryonic yolk sacs of embryos or fetal material. For example, such cells can be derived from primordial germ cells (PGCs)

Pluripotential stem cells can be derived and maintained using standard methods for pluripotent stem cells except as provided herein. Methods for producing pluripotent cells, including EG cells, are disclosed in U.S. Pat. No. 5,690,926 by Hogan and methods for producing EG cells are disclosed in U.S. Pat. No. 6,562,619 by Gearhart et al, which are hereby incorporated by reference herein in their entirety.

Pluripotential stem cells can also be derived from early embryos, such as blastocysts, testes (fetal and adult), and from other pluripotent stem cells such as ES and EG cells following the methods and using the compositions described herein.

Pluripotential stem cells can be produced from the fetal material from any animal, such as any mammal. However, in one aspect, the mammal is a rodent, such as a mouse, guinea pig, or rat. The fetal material can be from livestock, such as cattle, horses, pigs, sheep, goats, etc. The fetal material can be from primates, including humans. The methods and compositions described herein are utilized but non-human animal, e.g. mouse, guinea pig, or rat cattle, horses, pigs, sheep, goats, monkeys, apes, non-human primates, is substituted for the human embryonic material. The non-human material can specifically not be mouse or other rodents. Thus, non-human pluripotent stem cells, e.g., mouse, guinea pig, or rat cattle, horses, pigs, sheep, goats, monkeys, apes, non-human primates, are provided which are SSEA4 negative, positive for nonog, positive for Sox2, and positive for Oct4. These non-human pluripotent cells can also be positive for alkaline phosphatase, positive for TRA-1-60, positive for TRA-1-81, negative for nestin, and/or positive for SSEA3. The cells can maintain the potential to differentiate into derivatives if endodermal, mesodermal, and ectodermal cells. The cells can also maintain a normal karyotype through prolonged culture.

Pluripotent stem cell lines have also been reported for example in chicken (Pain, B., Clark, M. E., Shen, M., Nakazawa, H., Sakurai, M., Samarut, J. & Etches, R. J. (1996) Development (Cambridge, U.K.) 122, 2339-2348), mink (Sukoyan, M. A., Vatolin, S. Y., Golubitsa, A. N., Zhelezova, A. I., Semenova, L. A. & Serov, O. L. (1993) Mol. Reprod. Dev. 36, 148-158), hamster (Doetschman, T., Williams, P. & Maeda, N. (1988) Dev. Biol. 127, 224-227), pig (Wheeler, M. B. (1994) Reprod. Fertil. Dev. 6, 563-568; Shim, H., Gutierrez-Adan, A., Chen, L., BonDurant, R., Behboodi, E. & Anderson, G. (1997) Biol. Reprod. 57, 1089-1095), rhesus monkey (Thomson, J. A., Kalishman, J., Golos, T. G., Durning, M., Harris, C. P., Becker, R. A. & Hearn, J. P. (1995) Proc. Natl. Acad. Sci. USA 92, 7844-7848), and common marmoset (Thomson, J. A., Kalishman, J., Golos, T. G., Durning, M., Harris, C. P. & Hearn, J. P. (1996) Biol. Reprod. 55, 254-259); all of the preceding references are hereby incorporated by reference in their entirety for the teachings relating to the derivation of stem cell lines.

2. PCs™ (Pluricells)

Provided herein are compositions and methods for the derivation of a pluripotent stem cell, which is herein referred to as a PC. As disclosed herein, PCs are alkaline phosphatase (AP) positive, SSEA-1 positive, and SSEA-4 negative. PCs can also be nanog positive, Sox2 positive, and Oct-4 positive. PCs can also be Tcl1 positive, and Tbx3 positive. PCs can also be Cripto positive, Stellar positive and Daz1 positive. PCs also can express cell surface antigens that bind with antibodies having the binding specificity of monoclonal antibodies. TRA-1-60 (ATCC HB-4783) and TRA-1-81 (ATCC HB-4784). PCs are capable of differentiating into derivatives of endodermal, mesodermal, and ectodermal cells throughout the culture. Further, as disclosed herein, these properties of PCs can be maintained without a feeder layer for at least 10, 11, 12, 13, 14, 15, 16, 17, 18, 19, 20 passages or for over a year. PCs can be human or other animal. For example, PCs can be mouse, guinea pig, rat. cattle, horses, pigs, sheep, goats, etc. PCs can also be from non-human primates.

Also as provided herein, PCs can be differentiated into multipotent cells (e.g., progenitors) or into more terminally differentiated cells such as heart, liver, neural, pancreatic islet, or virtually any cell of the body.

PCs can be isolated from fetal material, for example, from gonadal tissues, genital ridges, mesenteries or embryonic yolk sacs of embryos or fetal material. For example, such cells can be derived from primordial germ cells (PGCs)

PCs can be derived and maintained using standard methods for pluripotent stem cells except as provided herein. Methods for producing pluripotent cells, including EG cells, are disclosed in U.S. Pat. No. 5,690,926 by Hogan and methods for producing EG cells are disclosed in U.S. Pat. No. 6,562,619 by Gearhart et al, which are hereby incorporated by reference herein in their entirety.

PCs can also be derived from early embryos, such as blastocysts, testes (fetal and adult), and from other pluripotent stem cells such as ES and EG cells following the methods and using the compositions described herein.

PCs can be produced from the fetal material from any animal, such as any mammal. However, in one aspect, the mammal is a rodent, such as a mouse, guinea pig, or rat. The fetal material can be from livestock, such as cattle, horses, pigs, sheep, goats, etc. The fetal material can be from primates, including humans. The methods and compositions described herein are utilized but non-human animal, e.g. mouse, guinea pig, or rat cattle, horses, pigs, sheep, goats, monkeys, apes, non-human primates, is substituted for the human embryonic material. The non-human material can specifically not be mouse or other rodents. Thus, non-human pluripotent stem cells, e.g., mouse, guinea pig, or rat cattle, horses, pigs, sheep, goats, monkeys, apes, non-human primates, are provided which are SSEA4 negative, positive for nonog, positive for Sox2, and positive for Oct4. These non-human pluripotent cells can also be positive for alkaline phosphatase, positive for TRA-1-60, positive for TRA-1-81, negative for nestin, and/or positive for SSEA3. The cells can maintain the potential to differentiate into derivatives if endodermal, mesodermal, and ectodermal cells. The cells can also maintain a normal karyotype through prolonged culture.

Pluripotent stem cell lines have also been reported for example in chicken (Pain, B., Clark, M. E., Shen, M., Nakazawa, H., Sakurai, M., Samarut, J. & Etches, R. J. (1996) Development (Cambridge, U.K.) 122, 2339-2348), mink (Sukoyan, M. A., Vatolin, S. Y., Golubitsa, A. N., Zhelezova, A. I., Semenova, L. A. & Serov, O. L. (1993) Mol. Reprod. Dev. 36, 148-158), hamster (Doetschman, T., Williams, P. & Maeda, N. (1988) Dev. Biol. 127, 224-227), pig (Wheeler, M. B. (1994) Reprod. Fertil. Dev. 6, 563-568; Shim, H., Gutierrez-Adan, A., Chen, L., BonDurant, R., Behboodi, E. & Anderson, G. (1997) Biol. Reprod. 57, 1089-1095), rhesus monkey (Thomson, J. A., Kalishman, J., Golos, T. G., Durning, M., Harris, C. P., Becker, R. A. & Hearn, J. P. (1995) Proc. Natl. Acad. Sci. USA 92, 7814-7848), and common marmoset (Thomson, J. A., Kalishman, J., Golos, T. G., Durning, M., Harris, C. P. & Hearn, J. P. (1996) Biol. Reprod. 55, 254-259); all of the preceding references are hereby incorporated by reference in their entirety for the teachings relating to the derivation of stem cell lines.

Also disclosed herein are PCs derived without the use of a feeder layer and a method of producing such cell. PCs can be isolated directly on a solid substrate, e.g. plastic, glass or the like, and can be maintained without the need of a feeder layer. As disclosed herein, PCs are alkaline phosphatase (AP) positive, SSEA-1 positive, and SSEA-4 negative. PCs can also be nanog positive and Oct-4 positive. PCs can also be Tcl1 positive, and Tbx3 positive. PCs can also be Cripto positive, Stellar positive and Daz1 positive. PCs also can express cell surface antigens that bind with antibodies having the binding specificity of monoclonal antibodies TRA-1-60 (ATCC HB-4783) and TRA-1-81 (ATCC HB-4784). PCs are capable of differentiating into derivatives of endodermal, mesodermal, and ectodermal cells throughout the culture. Further, as disclosed herein, these properties of PCs can be maintained without a feeder layer for at least 10, 11, 12, 13, 14, 15, 16, 17, 18, 19, 20 passages. Also as provided herein, the cells can be differentiated into more differentiated cell types, e.g. multipotent cells such as hematopoietic stem cells or more terminally differentiated cells such as heart, liver, neural, pancreatic islet, or virtually any cell of the body. As disclosed herein, PCs can be grown on either a feeder layer or directly on a solid substrate without the use of a feeder layer or medium conditioned by a feeder layer.

The disclosed stem cells, such as PCs, that were derived and maintained on a solid substrate such as plastic and have subsequently never been exposed to a feeder layer, are distinct from stem cells that were isolated and grown on feeder layers. For example, the PCs can be negative for Neu5Gc sialic acid and not elicit an immune reponse of antibodies specific for Neu5Gc. Sialic acids are a family of acidic sugars displayed on the surfaces of all cell types, and on many secreted proteins. The two most common mammalian sialic acids are N-glycolylneuraminic acid (Neu5Gc) and N-acetylneuraminic acid (Neu5Ac), with Neu5Ac being the metabolic precursor of Neu5Gc. Humans are genetically unable to produce Neu5Gc from Neu5Ac. Thus, although human cells have no overall loss of sialic acids, they express primarily Neu5Ac. But they can potentially take Neu5Gc up from media containing animal products, activate it into CMP-Neu5Gc, and metabolically incorporate it using the same Golgi transporter and sialyltransferases as CMP-Neu5Ac. Most normal healthy humans have circulating antibodies specific for Neu5Gc. Thus, xenogenic culture methodology can compromise transplantation success, resulting from uptake and expression of Neu5Gc on the surface of any tissue developed from HESC. Such incorporation can induce an immune response upon transplantation. Thus, as disclosed herein, PCs can be isolated and maintained in medium not containing Neu5Gc. For example, the medium can lack non-human, animal products. Such medium and cells are provided herein.

Provided herein are PCs that can be maintained without the need for a feeder layer. Also provided herein are PCs that were isolated directly on a solid substrate such as plastic, glass or the like and can be maintained without the need of a feeder layer. Also provided herein is a composition comprising PCs contacting a solid substrate without a feeder layer, wherein the cells can be maintained on the substrate for at least 10, 11, 12, 13, 14, 15, 16, 17, 18, 19, 20 passages or passaged for over a year.

The solid substrate can be plastic, such as tissue culture plastic. As used herein, “tissue culture plastic” includes polystyrene that has been rendered wettable by oxidation, a treatment that increases its adhesiveness for cells from animal tissues and without which anchorage dependent cells will not grow. The solid substrate can be dishes, flasks, multiwell plates, etc. Other suitable substrates for growing cells in culture are known in the art and can be used to grown PCs as described herein. In one aspect, the solid substrate has a charged surface to allow adhesion of the cells. The surface charge can be produced by coating a solid substrate with certain proteins known in the art. For example, solid substrates such as glass can be coated with a Poly-D-Lysine, gelatin, or with a matrix protein, such as, for example, fibronectin, laminin, or Matrigel®. However, substrate coatings are not required to derive or maintain the growth of PCs without differentiation. Thus, the herein disclosed PCs can be isolated and/or maintained on a solid substrate that is not coated with a matrix protein.

The undifferentiated growth of PCs can be dependent upon stem cell factor (SCF). The undifferentiated growth of PCs can be dependent upon oncostatin M. The undifferentiated growth of PCs can be independent of IL-6, ciliary neurotrophic factor, and/or LIF.

The disclosed PCs can be produced by a method comprising culturing pluripotent stem cells in a culture medium, wherein the culture medium comprises a base medium suitable for growing stem cells and amounts of oncostatin M and stem cell factor (SCF) sufficient to maintain the stem cell without a feeder layer for at least 20 passages or for over a year.

PCs can also be produced by a method comprising providing primordial germ cells (PGCs) from a human embryo; culturing the primordial germ cells on a solid substrate in a culture medium; selecting cells that exhibit the following characteristics: maintains a normal karyotype for at least 20 passages and maintains the potential to differentiate into derivatives of endodermal, mesodermal, and ectodermal cells throughout the culture; and isolating said pluripotent human stem cells, wherein the culture medium comprises a base medium suitable for growing stem cells and oncostatin M sufficient to maintain the stem cell without a feeder layer for at least 20 passages.

As disclosed herein, PCs are capable of differentiating into derivatives of endodermal, mesodermal, and ectodermal cells throughout the culture. Further, as disclosed herein, these properties of PCs can be maintained without a feeder layer for at least 10, 11, 12, 13, 14, 15, 16, 17, 18, 19, 20 passages or for over a year. Also as provided herein, the cells can be differentiated into more differentiated cell types, e.g. multipotent cells such as hematopoietic stem cells or more terminally differentiated cells such as heart, liver, neural, pancreatic islet, or virtually any cell of the body.

3. Feeder Layer-Independent Stem Cells

Disclosed herein are stem cells that can be derived without use of and/or contact with a feeder layer. The disclosed stem cells can be maintained without use of and/or contact with a feeder layer. The disclosed stem cells can be derived and maintained without use of and/or contact with a feeder layer. The disclosed stem cells can be maintained and/or grown on a solid substrate such as plastic, glass and the like without a feeder layer. The disclosed stem cells can be derived without use of and/or contact with conditioned media. The disclosed stem cells can be maintained without use of and/or contact with conditioned media. The disclosed stem cells can be derived and maintained without use of and/or contact with conditioned media. The disclosed stem cells can be derived without use of and/or contact with a feeder layer or conditioned media. The disclosed stem cells can be maintained without use of and/or contact with a feeder layer or conditioned media. The disclosed stem cells can be derived and maintained without use of and/or contact with a feeder layer or conditioned media. The disclosed stem cells can be negative for N-glycolylneuraminic acid (Neu5Gc). The disclosed stem cells can be derived without use of and/or contact with N-glycolylneuraminic acid (Neu5Gc). The disclosed stem cells can be maintained without use of and/or contact with N-glycolylneuraminic acid (Neu5Gc). The disclosed stem cells can be derived and maintained without use of and/or contact with N-glycolylneuraminic acid (Neu5Gc). The disclosed stem cells can be negative for carbohydrates not produced in humans or by human cells. The disclosed stem cells can be derived without use of and/or contact with carbohydrates not produced in humans or by human cells. The disclosed stem cells can be maintained without use of and/or contact with carbohydrates not produced in humans or by human cells. The disclosed stem cells can be derived and maintained without use of and/or contact with carbohydrates not produced in humans or by human cells.

The disclosed stem cells can be derived, for example, from a primordial germ cell (PGC), blastocyst, epiblast, gonadal ridge, teste, or embryo. The disclosed stem cells can be also be derived from other stem cells, such as multipotent stem cells (e.g., adult stem cells, MAPCs, MSCs, and HSCs), pluripotential stem cells (e.g., ES cells, EG cells, PC cells, and EC cells), and OISCs. The stem cell can stain positive for the SSEA-1 antigen, stain negative for SSEA-4 antigen, and/or stain positive for alkaline phosphatase. The disclosed stem cells can be positive for Oct-4, positive for nanog, positive for Tcl1, positive for Tbx3, positive for Cripto, positive for Stellar, positive for Daz1, positive for SSEA3, positive for TRA-1-60, and/or positive for TRA-1-81. The stem cell can be in contact with a solid substrate such as plastic, glass, or the like. The stem cell can be a clone. The solid substrate can be plastic.

The disclosed stem cells can stain negative for the SSEA-4 antigen. Thus, at least 50, 60, 70, 80, 90, 91, 92, 93, 94, 95, 96, 97, 98, 99% of the disclosed stem cells can stain negative for the SSEA-4 antigen. The disclosed stem cells can stain positive for the SSEA-1 antigen. Thus, at least 50, 60, 70, 80, 90, 91, 92, 93, 94, 95, 96, 97, 98, 99% of the disclosed stem cells can stain positive for the SSEA-1 antigen. The disclosed stem cells can maintain a normal karyotype. The cell can maintain the potential to differentiate into derivatives of endodermal, mesodermal, and ectodermal cells throughout the culture. The disclosed stem cells can stain positive for alkaline phosphatase. Thus, at least 50, 60, 70, 80, 90, 91, 92, 93, 94, 95, 96, 97, 98, 99% of the disclosed stem cells can stain positive for alkaline phosphatase. The disclosed stem cells can be derived from a primordial germ cell (PGC). The disclosed stem cells can stain negative for Neu5Gc. A composition is provided comprising an isolated pluripotent stem cell which stains negative for the SSEA-4 antigen and at least 1 uM of oncostatin M and can have one or more of the above characteristics.

For example, disclosed is an isolated pluripotent stem cell that can be maintained without a feeder layer for at least 20 passages, wherein the cell maintains the potential to differentiate into derivatives of endodermal, mesodermal, and ectodermal cells throughout the culture, stains negative for SSEA-4 antigen, and maintains a normal karyotype.

Also disclosed is an isolated pluripotent stem cell which stains negative for the SSEA-4 antigen.

Also disclosed is an isolated pluripotent stem cell that maintains the potential to differentiate into derivatives of endodermal, mesodermal, and ectodermal cells throughout the culture; stains negative for SSEA-4 antigen; stains positive for the SSEA-1 antigen; stains positive for alkaline phosphatase; stains positive for Oct-4; and stains negative for nestin.

Also disclosed is an isolated pluripotent stem cell that maintains the potential to differentiate into derivatives of endodermal, mesodermal, and ectodermal cells throughout the culture; stains negative for SSEA-4 antigen; stains positive for the SSEA-1 antigen; stains positive for alkaline phosphatase; stains positive for Oct-4; stains negative for nestin; and can maintain a normal karyotype in prolonged culture.

Also disclosed herein are stem cells, such as multipotent stem cells (e.g., adult stem cells, MAPCs, MSCs, and HSCs), pluripotential stem cells (e.g., ES cells, EG cells, PC cells, and EC cells), and OISCs, derived without the use of a feeder layer and a method of producing such cell. The disclosed stem cells, such as multipotent stem cells (e.g., adult stem cells, MAPCs, MSCs, and HSCs), pluripotential stem cells (e.g., ES cells, EG cells, PC cells, and EC cells), and OISCs, that were derived and maintained on a solid substrate such as plastic and have subsequently never been exposed to a feeder layer, are distinct from stem cells that were isolated and grown on feeder layers. For example, the stem cells, such as multipotent stem cells (e.g., adult stem cells, MAPCs, MSCs, and HSCs), pluripotential stem cells (e.g., ES cells, EG cells, PC cells, and EC cells), and OISCs, can be negative for Neu5Gc sialic acid and not elicit an immune reponse of antibodies specific for Neu5Gc. Sialic acids are a family of acidic sugars displayed on the surfaces of all cell types, and on many secreted proteins. The two most common mammalian sialic acids are N-glycolylneuraminic acid (Neu5Gc) and N-acetylneuraminic acid (NeuSAc), with NeuSAc being the metabolic precursor of Neu5Gc. Humans are genetically unable to produce Neu5Gc from Neu5Ac. Thus, although human cells have no overall loss of sialic acids, they express primarily Neu5Ac. But they can potentially take Neu5Gc up from media containing animal products, activate it into CMP-Neu5Gc, and metabolically incorporate it using the same Golgi transporter and sialyltransferases as CMP-Neu5Ac. Most normal healthy humans have circulating antibodies specific for Neu5Gc. Thus, xenogenic culture methodology can compromise transplantation success, resulting from uptake and expression of Neu5Gc on the surface of any tissue developed from HESC. Such incorporation can induce an immune response upon transplantation. Thus, as disclosed herein, stem cells, such as multipotent stem cells (e.g., adult stem cells, MAPCs, MSCs, and HSCs), pluripotential stem cells (e.g., ES cells, EG cells, PC cells, and EC cells), and OISCs, can be isolated and maintained in medium not containing Neu5Gc. For example, the medium can lack non-human, animal products. Such medium and cells are provided herein.

Provided herein are stem cells, such as multipotent stem cells (e.g., adult stem cells, MAPCs, MSCs, and HSCs), pluripotential stem cells (e.g., ES cells, EG cells, PC cells, and EC cells), and OISCs, that can be maintained without the need for a feeder layer. Also provided herein are stem cells, such as multipotent stem cells (e.g., adult stem cells, MAPCs, MSCs, and HSCs), pluripotential stem cells (e.g., ES cells, EG cells, PC cells, and EC cells), and OISCs, that were isolated directly on a solid substrate such as plastic, glass or the like and can be maintained without the need of a feeder layer. Also provided herein is a composition comprising stem cells, such as multipotent stem cells (e.g., adult stem cells, MAPCs, MSCs, and HSCs), pluripotential stem cells (e.g., ES cells, EG cells, PC cells, and EC cells), and OISCs, contacting a solid substrate without a feeder layer, wherein the cells can be maintained on the substrate for at least 10, 11, 12, 13, 14, 15, 16, 17, 18, 19, 20 passages or passaged for over a year.

The solid substrate can be plastic, such as tissue culture plastic. As used herein, “tissue culture plastic” includes polystyrene that has been rendered wettable by oxidation, a treatment that increases its adhesiveness for cells from animal tissues and without which anchorage dependent cells will not grow. The solid substrate can be dishes, flasks, multiwell plates, etc. Other suitable substrates for growing cells in culture are known in the art and can be used to grow stem cells as described herein. In one aspect, the solid substrate has a charged surface to allow adhesion of the cells. The surface charge can be produced by coating a solid substrate with certain proteins known in the art. For example, solid substrates such as glass can be coated with a Poly-D-Lysine, gelatin, or with a matrix protein, such as, for example, fibronectin, laminin, or Matrigel®. However, substrate coatings are not required to derive or maintain the growth of stem cells, such as multipotent stem cells (e.g., adult stem cells, MAPCs, MSCs, and HSCs), pluripotential stem cells (e.g., ES cells, EG cells, PC cells, and EC cells), and OISCs. Thus, the herein disclosed stem cells, such as multipotent stem cells (e.g., adult stem cells, MAPCs, MSCs, and HSCs), pluripotential stem cells (e.g., ES cells, EG cells, PC cells, and EC cells), and OISCs, can be isolated and/or maintained on a solid substrate that is not coated with a matrix protein.

The undifferentiated growth of stem cells, such as multipotent stem cells (e.g., adult stem cells, MAPCs, MSCs, and HSCs), pluripotential stem cells (e.g., ES cells, EG cells, PC cells, and EC cells), and OISCs, can be dependent upon stem cell factor (SCF). The undifferentiated growth of the stem cells, such as multipotent stem cells (e.g., adult stem cells, MAPCs, MSCs, and HSCs), pluripotential stem cells (e.g., ES cells, EG cells, PC cells, and EC cells), and OISCs, can be dependent upon oncostatin M. The undifferentiated growth of the stem cells, such as multipotent stem cells (e.g., adult stem cells, MAPCs, MSCs, and HSCs), pluripotential stem cells (e.g., ES cells, EG cells, PC cells, and EC cells), and OISCs, can be independent of IL-6, ciliary neurotrophic factor, amd/or LIF.

The disclosed stem-cells, such as multipotent stem cells (e.g., adult stem cells, MAPCs, MSCs, and HSCs), pluripotential stem cells (e.g., ES cells, EG cells, PC cells, and EC cells), and OISCs, can be produced by a method comprising culturing pluripotent stem cells in a culture medium, wherein the culture medium comprises a base medium suitable for growing stem cells and amounts of oncostatin M and stem cell factor (SCF) sufficient to maintain the stem cell without a feeder layer for at least 20 passages or for over a year.

4. Stem Cell Culture Medium

The disclosed stem cell culture medium comprises a suitable amount of oncostatin M and stem cell factor (SCF) sufficient to maintain the stem cell without a feeder layer for at least 20 passages. The stem cell culture medium can also comprise a suitable amount of foreskolin, or a factor that elevates intracellular cAMP, sufficient to maintain the stem cell without a feeder layer for at least 20 passages. The stem cell culture medium can comprise an amount of a suitable FGF (e.g. FGF-2) sufficient to maintain the stem cell without a feeder layer for at least 20 passages. Thus, the stem cell culture medium can comprise at least 5 uM forskolin. The stem cell culture medium can comprise at least 5 ng per ml FGF (e.g. FGF-2). The stem cell culture medium can comprise at least 5 ng per ml stem cell factor (SCF). The stem cell culture medium can comprise at least 1 uM of oncostatin M.

Thus, the provided stem cell culture medium can be any base medium further comprising oncostatin M. Oncostatin M can be produced by lymphoid cells. There are two oncostatin M-related proteins (Mr 36,000 and 32,000) secreted by COS cells transfected with oncostatin M cDNA. The two proteins are described in detail in Martin M J, Nat Med. 2005 February; 11(2):228-32, which is incorporated herein in its entirety for the teaching of oncostatin M proteins. The smaller of these forms lacked a hydrophilic C-terminal domain comprising predominantly basic amino acids. The 32,000-Mr (short) form of oncostatin M is derived from the 227-amino-acid propeptide by proteolytic cleavage at or near the paired basic residues at positions 195 and 196. Propeptide processing of oncostatin M may be important for regulating in vivo activities of this cytokine. The provided stem cell culture medium can comprise the long (Mr 36,000) and/or short (Mr 32,000) oncostatin M. The nucleic acid sequence for oncostatin M can be found at GenBank Accession No. NM_{—}020530. Sequences and vectors comprising same are described in Malik N, et al. Mol Cell Biol. 1989 July; 9(7):2847-53, which is incorporated herein in its entirety for the teaching of oncostatin M proteins.

The base medium can be any medium suitable for growing stem cells. For example, the base medium can be Dulbecco's modified Eagle's medium (DMEM) or Knockout DMEM (Invitrogen). The medium can contain retinoic acid and essential vitamins. The medium can contain about 5%, 10%, 15%, 20% serum or serum replacements (e.g. knockout serum replacement; Invitrogen). In one aspect, the serum does not contain non-human animal products. In another aspect, the serum is human serum. In another aspect, the medium can be a serum-free defined medium. An example of the ingredients of a defined medium are provided in Table 1.

TABLE 1
Defined Medium

| Ingredient | (mg/L) | Ingredient | (mg/L) |
|---|---|---|---|
| Ammonium Molybdate | 0.008 | Tyrosine | 28 |
| Calcium chloride | 121 | Valine | 42 |
| Cobalt chloride | 0.007 | ascorbic acid | 25 |
| Copper sulfate | 0.018 | biotin | 0.17 |
| Ferrous nitrate | 0.427 | pantothenate | 0.53 |
| Magnesium sulfate | 354 | choline chloride | 13.5 |
| Manganese sulfate | 0.005 | ergocalciferol | 0.03 |
| Potassium chloride | 257 | folic acid | 0.75 |
| Sodium chloride | 6400 | forskolin | 4.1 |
| Sodium bicarbonate | 1872 | inositol | 6.7 |
| Sodium phosphate | 306 | Linoleic acid | 10 |
| Sodium selenite | 0.017 | Menadione | 0.01 |
| Zinc Sulfate | 0.297 | Nicotinamide | 0.4 |
| alanine | 9 | Pyridoxal | 0.4 |
| Arginine | 50 | Riboflavin | 0.1 |
| asparagine | 40 | Tocopherol | 0.003 |
| aspartic | 50 | Thiamine | 0.8 |
| cysteine | 25 | Vitamin A | 0.03 |
| cystine | 12 | Vitamin B12 | 0.1 |
| glutamic | 15 | Glutathione | 8 |
| glutamine | 87 | Hypoxanthine | 2.2 |
| glycine | 36 | Lipoic | 0.07 |
| histidine | 15 | Putrescine | 0.05 |
| isoleucine | 26 | Pyruvate | 45 |
| leucine | 46 | Thymidine | 0.24 |
| lysine | 122 | Glucose | 2000 |
| methionine | 23 | Human Insulin | 1 |
| phenylalanine | 27 | Human transferrin | 100 |
| proline | 21.5 | Human serum albumin | 5000 |
| Serine | 55 | Human oncostatin M | 0.01 |
| Threonine | 42 | Human stem cell | 0.01 |
| Tryptophan | 17 | factor | |
| | | Human FGF-2 | 0.025 |

The provided stem cell culture medium can comprise forskolin or factor that elevates intracellular cAMP sufficient to culture and maintain stem cells. For example, the disclosed culture medium can comprise at least about 1, 2, 3, 4, 5, 6, 7, 8, 9, 10 uM or more forskolin. As disclosed herein, absent forskolin, stem cells grown directly on plastic grow as clusters.

It should be recognized that FGF, SCF, and oncostatin M are all proteins and as such certain modifications can be made to the proteins which are silent and do not remove the activity of the proteins as described herein. Such modifications include additions, substitutions and deletions. Methods modifying proteins are well established in the art (Sambrook et al., Molecular Cloning: A Laboratory Manual, 2nd Ed., Cold Spring Harbor Laboratory, Cold Spring Harbor, N.Y., 1989).

For example, 1 liter of the stem cell culture medium can comprise DMEM (e.g. Knockout DMEM; Invitrogen), about 15% serum or serum replacement, about 10 ng per ml oncostatin M, about 10 ng/ml human stem cell factor, about 10-25 ng/ml human FGF (e.g. FGF-2), about 10 uM forskolin, about 1 mM glutamine, about 0.1 M mercaptoethanol, and about 0.1 mM non-essential amino acids. Other ingredients and modifications that can be made to the provided medium that are suitable for culturing stem cells are known in the art and are contemplated herein.

a) Oncostatin

For example, the stem cell culture medium can comprise at least about 1, 2, 3, 4, 5, 6, 7, 8, 9, 10 ng per ml oncostatin M. As disclosed herein, absent oncostatin M, stem cells grown directly on plastic stop dividing and die within about a day. Oncostatin M and mutants thereof, as well as methods for their preparation, are described in detail in U.S. Pat. Nos. 5,120,535, 5,428,012, and 5,874,536, which are hereby incorporated herein by reference in their entirety for these teachings. Oncostatin M, as used herein, includes natural forms, including such forms produced in mammals, such as humans, as well as homologues and mutants thereof. Oncostatin M can be obtained by any method, and includes the use of modified or truncated Oncostatin molecules and Oncostatin M analogs which retain the desired activity.

The nucleic acid sequence for human oncostatin M can be found at GenBank Accession No. NM_{—}020530 and the corresponding amino acid sequence can be found at Accession No. NP_{—}065391. For example, oncostatin M for use in the herein disclosed compositions and methods can comprise a polypeptide having at least 70, 75, 80, 85, 90, 95, 100% sequence identity to the amino acid sequence set forth in Accession No. NP_{—}065391.

Oncostatin M may be obtained by techniques well known in the art from a variety of cell sources which synthesize bioactive Oncostatin M including, for example, cells which naturally produce Oncostatin M and cells transfected with recombinant DNA molecules capable of directing the synthesis and/or secretion of Oncostatin M. Alternatively, Oncostatin M may be synthesized by chemical synthetic methods including but not limited to solid phase peptide synthesis.

b) SCF

The provided stem cell culture medium can comprise stem cell factor (SCF), including human SCF sufficient to culture and maintain stem cells. For example, the culture medium can comprise at least about 1, 2, 3, 4, 5, 6, 7, 8, 9, 10 ng or more per ml SCF. As disclosed herein, absent SCF, stem cells grown directly on plastic can fail to attach to the dish. Stem cell factor (SCF) is also called steel factor, mast cell growth factor and c-kit ligand in the art. SCF is a transmembrane protein with a cytoplasmic domain and an extracellular domain. SCF is well known in the art; see European Patent Publication No. 0 423 980 A1, corresponding to European Application No. 90310889.1.

Stem cell factor (SCF) is an early acting hematopoietic factor. The purification, cloning and use of SCF have been reported in U.S. Pat. No. 6,204,363, which is incorporated herein by reference in its entirety for this teaching. SCF, as used herein, includes natural forms, including such forms produced in mammals, such as humans, as well as homologues and mutants thereof. SCF can be obtained by any method, and includes the use of modified or truncated SCF molecules and SCF analogs which retain the desired activity.

The nucleic acid sequence for human stem cell factor (SCF) can be found at GenBank Accession No. NM_{—}000899 and the corresponding amino acid sequence can be found at Accession No. NP_{—}000890. For example, SCF for use in the herein disclosed compositions and methods can comprise a polypeptide having at least 70, 75, 80, 85, 90, 95, 100% sequence identity to the amino acid sequence set forth in Accession No. NP_{—}000890.

SCF may be obtained by techniques well known in the art from a variety of cell sources which synthesize bioactive SCF including, for example, cells which naturally produce SCF and cells transfected with recombinant DNA molecules capable of directing the synthesis and/or secretion of SCF. Alternatively, SCF may be synthesized by chemical synthetic methods including but not limited to solid phase peptide synthesis.

c) FGF

The provided stem cell culture medium can comprise a growth factor, such as fibroblast growth factor (e.g. FGF-2), sufficient to culture and maintain stem cells. For example, the disclosed culture medium can comprise at least about 1, 2, 3, 4, 5, 6, 7, 8, 9, 10 ng or more per ml FGF-2. As disclosed herein, absent FGF, stem cells grown directly on plastic can stop dividing.

A “fibroblast growth factor” (FGF) as used herein means any FGF suitable for culturing pluripotent stem cells. There are presently at least 23 known FGFs (Yamaguchi et al. (1992)). These FGFs include FGF-1 (acidic fibroblast growth factor), FGF-2 (basic fibroblast growth factor), FGF-3 (int-2), FGF-4 (hst/K-FGF), FGF-5, FGF-6, FGF-7 and FGF-8, and so on. Each of the suitable factors can be utilized directly in the methods taught herein to produce or maintain stem cells. Each FGF can be screened in the methods described herein to determine if the FGF is suitable to enhance the growth of or allow continued proliferation of stem cells or their progenitors. Various examples of FGF and methods of producing an FGF are well known; see, for example, U.S. Pat. Nos. 4,994,559; 4,956,455; 4,785,079; 4,444,760; 5,026,839; 5,136,025; 5,126,323; and 5,155,214.

d) Cells Grown in Medium

Also provided herein are cells grown in the disclosed culture medium. Thus, provided are stem cells grown in a culture medium comprising Oncostatin. Also disclosed are stem cells grown in a culture medium comprising Oncostatin and Stem Cell Factor (SCF). The disclosed stem cells can be derived, for example, from a primordial germ cell (PGC), blastocyst, epiblast, gonadal ridge, teste, or embryo. Alternatively, the disclosed stem cells can be derived from an adult cell, such as, for example, an multipotential adult progenitor cell (MAPC), Mesenchymal Stem Cell (MSC), or Hematopoietic Stem Cell (HSC).

Thus, disclosed herein is a stem cell produced by the method comprising culturing a PGC, gonadal ridge, teste, or embryo in a culture medium comprising at least about 1, 2, 3, 4, 5, 6, 7, 8, 9, 10 ng per ml. oncostatin M. Also provided is a stem cell produced by the method comprising culturing a PGC, gonadal ridge, teste, or embryo in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF.

Also disclosed herein is a stem cell produced by the method comprising culturing a blastocyst or epiblast in a culture medium comprising at least about 1, 2, 3, 4, 5, 6, 7, 8, 9, 10 ng per ml oncostatin M. Also provided is a stem cell produced by the method comprising culturing a blastocyst or epiblast in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF.

Also disclosed herein is a stem cell produced by the method comprising culturing a MAPC, MSC, or HSC in a culture medium comprising at least about 1, 2, 3, 4, 5, 6, 7, 8, 9, 10 ng per ml oncostatin M. Also provided is a stem cell produced by the method comprising culturing a MAPC, MSC, or HSC in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF.

5. Nestin-Positive Stem Cells

As disclosed herein, the removal of oncostatin and stem cell factor (SCF) from stem cells grown in the disclosed stem cell culture medium results in the formation of a substantially homogenous population of stem cells. By substantially homogenous is meant the cells are at least 90% of the cell type. The cells can also be that at least 50, 60, 70, 80, 90, 91, 92, 93, 94, 95, 96, 97, 98, 99%, 100% having the disclosed properties. As disclosed herein, the stem cells are nestin positive, Oct4 positive, and alkaline phosphatase (AP) negative. The stem cells can also be Sox2 positive, Nanog positive, alkaline phosphatase (AP) negative, SSEA-1 positive, and SSEA-4 negative. The stem cells can also be oncostatin independent. By “oncostatin independent” is meant the cells are cultured in the substantial functional absence of oncostatin. The stem cells can also be LIF independent. Thus, the removal of oncostatin and SCF from pluripotent stem cells grown in the disclosed stem cell culture medium can result in a population of cells wherein at least 50, 60, 70, 80, 90, 91, 92, 93, 94, 95, 96, 97, 98, 99, 100% of the cells are nestin positive, Oct4 positive, and alkaline phosphatase (AP) negative. The stem cells can also be oncostatin independent stem cells (OISC). As disclosed herein, OISCs are nestin positive, Oct4 positive, and alkaline phosphatase (AP) negative. OISCs can also be Nanog positive, Sox2 positive, alkaline phosphatase (AP) negative, SSEA-1 positive, and SSEA-4 negative.

Thus, disclosed herein is a stem cell produced by the method comprising:
(a) culturing a PGC, gonadal ridge, teste, or embryo in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF for at least 1, 2, 3, 4, 5, 6, 7, 8, 9, 10 passages;
(b) culturing said cells in medium comprising at least 1, 2, 3, 4, 5, 6, 7, 8, 9, 10 ng per ml FGF and less than 1 ng per ml oncostatin M and SCF;
(c) selecting cells that stains positive for alkaline phosphatase, SSEA-1, Oct-4, and Nestin; and
(d) isolating said stem cell.

Thus, disclosed herein is a stem cell produced by the method comprising:
(a) culturing a blastocyst or epiblast in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF for at least 1, 2, 3, 4, 5, 6, 7, 8, 9, 10 passages;
(b) culturing said cells in medium comprising at least 1, 2, 3, 4, 5, 6, 7, 8, 9, 10 ng per ml FGF and less than 1 ng per ml oncostatin M and SCF;
(c) selecting cells that stain positive for alkaline phosphatase, SSEA-1, Oct-4, and Nestin; and
(d) isolating said stem cell.

Thus, disclosed herein is a stem cell produced by the method comprising:
(a) culturing a MAPC, MSC, or HSC in a culture medium comprising at least about 1, 2, 3, 4, 5, 6, 7, 8, 9, 10 ng per ml oncostatin M and at least about 1, 2, 3, 4, 5, 6, 7, 8, 9, 10 ng or more per ml SCF for at least 1, 2, 3, 4, 5, 6, 7, 8, 9, 10 passages;
(b) culturing said cells in medium comprising at least 1, 2, 3, 4, 5, 6, 7, 8, 9, 10 ng per ml FGF and less than 1 ng per ml oncostatin M and SCF;
(c) selecting cells that stains positive for alkaline phosphatase, SSEA-1, Oct-4, and Nestin; and
(d) isolating said stem cell.

The stem cells can be produced and maintained without using a medium conditioned by a cell line or feeder layer. Thus, in one aspect, the stem cells are not cultured in a conditioned medium. For example, the stem cells can be produced and maintained without using a medium conditioned by exposure to a hepatocellular carcinoma cell line, such as HepG2. Thus, in one aspect, The stem cells are not cultured in a medium conditioned by a hepatocellular carcinoma cell line.

The stem cells can be capable of differentiating into derivatives of endodermal, mesodermal, and ectodermal cells throughout the culture. For example, the stem cells can be directed to become progenitors such as, for example, myoblasts, hemangioblasts, or neural progenitor cells (NPCs). Said cells can also be directed to become more terminally differentiated cells such as muscle (cardiac, smooth, or skeletal), neural (neuron, oligodendrocyte, astrocyte), hematopoeitic, vascular, hepatic, pancreatic, or virtually any cell of the body. Further, as disclosed herein, these properties of the stem cells can be maintained without a feeder layer for at least 10, 11, 12, 13, 14, 15, 16, 17, 18, 19, 20 passages. Each of these cells can comprise at least 50, 60, 70, 80, 90, 91, 92, 93, 94, 95, 96, 97, 98, 99, 100% of the cells in culture. In addition, the cells, e.g. muscle (cardiac, smooth, or skeletal), neural (neuron, oligodendrocyte, astrocyte), hematopoeitic, vascular, hepatic, pancreatic, or virtually any cell type of the body can comprise at least 50, 60, 70, 80, 90, 91, 92, 93, 94, 95, 96, 97, 98, 99, 100% of the cells in culture in the absence of cell sorting.

Nestin is a class VI intermediate filament protein (Hockfield, S. et al. 1985; Lendahl, U. et al. 1990). Although it is expressed predominantly in stem cells of the central nervous system (CNS) (Frederiksen, K. et al. 1988), its expression is absent from nearly all mature CNS cells (Tohyama, T. et al. 1992). Nestin has been the most extensively used marker to identify CNS stem cells within various areas of the developing nervous system and in cultured cells in vitro (Uchida, N. et al. 2000; Frederiksen, K. et al. 1988; Cattaneo, C. et al. 1990). The role of nestin in CNS stem cell biology, however, remains undefined. Although nestin does not form intermediate filaments by itself in vitro a-internexin to form homo- and heterodimer, coiled-coil complexes that may then form intermediate filaments (Steinert, P. M. et al. 1999). Its transient expression has been suggested to be a major step in the neural differentiation pathway (Lendahl, U. et al. 1990). Nestin expression has also been discovered in non-neural stem cell populations, such as pancreatic islet progenitors (Zulewski, H. et al. 2001; Lumelsky, N. et al. 2001) as well as hematopoietic progenitors (Shih, C. C. et al. 2001). Nestin expression has also been detected in neuronal precursor cells, radial glia cells, Schwann cells, neural crest cells, oligodendrocyte precursors, developing skeletal muscle cells, developing cardiomyocytes, presomitic mesoderm, myotome, dermatome, mesonephric mesenchyme, myoid cells, dental lamina, dental epithelium, ectomesenchyme in dental papilla, enamel organ, dental follicle, stratum intermedium, pulp, endothelial cells of developing blood vessels, vascular endothelium of developing pancreas, pancreatic epithelial progenitor cells, epithelium of lens vesicle, retina (Müller cells), and hepatic oval cells.

6. Differentiation of Stem Cells in Vitro

In contrast to existing methods, the herein disclosed method of producing a homogenous population of progenitor cells from pluripotent stem cells does not require the formation of EBs. Instead, the removal of oncostatin M and stem cell factor (SCF) from the disclosed culture medium results in a homogenous population of nestin-positive stem cells that can be directed to a specific cell type using standard methods known in the art or disclosed herein. Thus, provided herein are compositions and methods for directed differentiation of stem cells into progenitor cells and/or terminally differentiated cells.

The term “directed differentiation” can refer to the manipulation of stem cell culture conditions to induce differentiation into a particular cell type. For example, pluripotent stem cells can be directed towards a specific lineage by 1) activating endogenous transcription factors; 2) transfection with ubiquitously expressing transcription factors; 3) exposure to selected growth factors; or 4) coculture of stem cells with cell types capable of lineage induction. Stem cells can be induced to form the lineage of interest by a combination of growth factors and/or their antagonists. These instructors of lineage formation accelerate differentiation in vitro and mimic the markers of natural developmental pathways. Thus, any composition or method known in the art or disclosed herein for directing differentiation can be used to produce cells from the disclosed stem cells, such as multipotent stem cells (e.g., adult stem cells, MAPCs, MSCs, and HSCs), pluripotential stem cells (e.g., ES cells, EG cells, PC cells, and EC cells), and OISCs.

a) Tissue Specific Reversible Transformation

Compositions and method for producing differentiated stem cells from pluripotent stem cells using reversible transformation is provided, for example, in U.S. patent application Ser. No. 11/194,143, which is hereby incorporated herein by reference in its entirety for the teaching of said compositions and methods. As used herein, “tissue specific reversible transformation” and “conditional immortalization” refer to the same method of combining tissue specific promoter/enhancers with reversible transforming genes to direct differentiation of cells.

Transformation is the process whereby a cell loses its ability to respond to the signals that would normally regulate its growth. This can take the form of a loss of function mutation, such as results in loss of a repressor of cell growth such as PTEN, or a gain of function mutation whereby a gene becomes permanently activated such as occurs in many RAS mutations. Many laboratories have shown that insertion of one or more of these transforming genes into a normal cell can free it of the usual constraints on its growth and allow it to proliferate (Downward, J. (2002) Nat. Rev. Cancer 3, 11-22). Reversible transformation activates the transforming gene in one instance, then shuts it off in another. There are several means to accomplish this reversal.

The combination of tissue specific promoter/enhancers with reversible transforming genes allows the identification and culture of any specific cell type from differentiating stem cells. This system provides the dual advantages referred to above in that it is general and can be used to generate large quantities of specific cell types. In fact, it allows the establishment of permanent, clonal or semi-purified, differentiated cell lines that can be characterized and frozen. Upon reversal, the entire population reverts, providing an unlimited source of characterized, differentiated, normal cells.

(1) Dominant Negative Reversal

Many transforming genes, such as RAS, have another known mutant that is a dominant negative. For example, dominant negative RAS sequesters RAF, another protein necessary for propagation of the RAS signal, such that RAS signaling is turned off (Fiordalisi, (2002) J Biol. Chem. 29, 10813-23). Using such activated/dominant negative pairs of genes provides a reversible system. Such pairs are known for RAS, SRC and p53, for example (Barone and Courtneidge, (1995) Nature. 1995 Nov. 30; 378(6556):509-12; Willis A, et al., Oncogene. 2004 Mar. 25; 23(13):2330-8).

(2) Temperature Sensitive Mutant Reversal

Another mechanism to effect reversible transformation is with temperature sensitive mutants (Jat, P S, et al., (1991) Proc. Natl. Acad. Sci. 88, 5096-5100). Temperature sensitive (ts) proteins are stable at the permissive temperature but unstable at the restrictive temperature. T antigen (TAg), the well known transforming gene of the SV40 virus, has several ts mutants. When tsTAg is inserted into a normal cell, the cell is transformed and proliferates at 32° C. but arrests and reverts to normal at 39° C. Several such temperature sensitive mutants are known for SV40 T antigen and adenovirus E1A, for example (Fahnestock, M L, Lewis, J B. (1989) J. Virol. 63, 2348-2351).

(3) Recombinase Reversal

A third mechanism for reversible transformation is to, in fact, reversibly insert the transforming gene. Cre/lox and flp/frt are two such mechanisms for reversible insertion (Sauer. B. (2002) Endocrine 19, 221-228; Schaft, J, et al., (2001) Genesis 31, 6-10). If a gene is transfected into a target cell capped on each end by lox recombination sites, treatment of the cell with CRE recombinase will excise the inserted sequence, leaving only a single lox sequence. Likewise, if a gene is transfected into a target call capped on each end by frt treatment with flp will excise the inserted sequence, leaving only the flp sequence.

Disclosed are compositions including cells that comprise one or more of the sequences disclosed herein, such as a cell comprising a transformation sequence driven by the insulin promoter, such as a purified or semi-purified or clonal population of cells comprising the recombinase sequence, such as a 10× or flp sequence, remaining after a recombination event, for example, wherein the cell was a cell previously containing one or more of the nucleic acids disclosed herein.

b) Molecule Directed Differentiation

The stem cells disclosed herein can also be directed to specific cell fates using molecules, such as, for example, drugs, prodrugs, peptides, and nucleic acids. Examples of molecules and methods for directing differentiation of stem cells to specific cell types are disclosed. However, other known or newly discovered molecules and strategies for directing cell fate can be applied to the stem cells provided herein.

The formation of ectodermal derivatives is very common in spontaneously differentiating stem cells and is commonly considered a developmental default pathway. The neural differentiating pathway can be enhanced in cultures to generate neural progenitors. These stem cell derived neurons can respond to neurotransmitters, generate action potentials, and make functional synapses (Carpenter M K, et al. 2001).

Oligodendrocytes can also be produced from stem cell culture using FGF (e.g. FGF-2) and epidermal growth factor (EGF), followed by the additional supplementation of retinoic acid (RA). The oligodendrocyte precursors produced are able to mature and remylinate neurons (Nistor G I, et al. 2005). Dopaminergic neurons can also be formed from stem cells (Park S, et al. 2004; Perrier A L, et al. 2004). Motor neurons can also be produced using the multistep method used for this differentiation pathway that utilises RA and FGF-2, then RA and sonic hedgehog (SHH), and finally brain-derived neurotrophic factor (BDNF), glial-derived neurotrophic factor (GDNF), insulin-like growth factor-1 (IGF1) and low levels of SHH.

The directed differentiation of stem cells into neuroectoderm can be efficiently achieved using Noggin, which is an antagonist to BMP signaling that is involved in the paracrine loop that drives stem cells into flattened epithelial which express genes characteristic of extra-embryonic endoderm. These cells are a human yolk sac cell type that proliferates in spontaneously differentiating cultures under the influence of BMP2 produced by stem cells. The “noggin cultures” are capable of renewal in culture as relatively homogeneous colonies of neuroectoderm and show facile conversion to neurons or glia in the appropriate culture systems (Pera M F, et al. 2004). On the other hand, prolonged culture of stem cells in serum-free medium with BMP4 will induce flat epithelial cells that express genes (e.g., MSX2), and proteins (e.g., human chorionic gonadotrophin) associated with trophoblast or placental development.

Coculture of stem cells with the mouse bone marrow mesenchymal PA6 cell line that produces stromal cell derived inducing activity (SDIA) will produce midbrain neuronal cells that are tyrosine hydrolase positive (TH+) and express nurr1 and LMX1b genes (Kawasaki H, et al. 2002; Mizuseki K, et al. 2003). In these differentiating cultures, pigmented retinal epithelium can also be recognized. Manipulation of culture conditions with BMP4 induces epidermogenesis or neural crest cells and dorsal-most central nervous system cells. Suppression of SHH promotes motor neuron formation (Trounson A. 2004). Stem cells can also be directed into midbrain dopamine neurons when grown with mouse bone marrow mesenchyme (MS5 and S2 cell lines), where there is sequential expression of the key transcription factors Pax2, Pax5 and engrailed-1 in response to a series of growth factors and patterning molecules (FGF-8, SHH, ascorpic acid and brain-derived neurotrophic factor-BDNF) (Perrier A L, et al. 2004).

Exposure of the FGF-2-expanded neuroepithelial cells of stem cell derivation, to FGF-8 and SHH, promotes differentiation of dopaminergic neurons with a forebrain phenotype, but early exposure to FGF-8 during neuroepithelial specification promotes the midbrain phenotype and subsequent midbrain dopaminergic neurons. Hence, the sequence of instruction by FGF-8 and SHH can determine the neuronal subtype.

Coculture methodologies have also been used to produce differentiated cardiomyocytes from stem cells. 15-20% of cultures of stem cells grown with the mouse visceral endoderm cell type END-2, form beating heart muscle colonies (Mummery C, et al. 2002; Mummery C, et al. 2003). Beating heart muscle cells derived from stem cells express cardiomyocyte markers including alpha-myosin heavy chain, cardiac troponins and atrial natriuretic factor as well as transcription factors typical of cardiomyocytes, eg. Nk×2.5, GATA4 and MEF3 (Kehat I, et al. 2001; Xu C, et al. 2002). These cells respond to pharmacological drugs and the action potentials of cardiomyocytes produced in this system most commonly resemble that for human fetal left ventricular cardiomyocytes, but are distinctly different to those of mouse cardiomyocytes (Mummery C, et al. 2003; He J Q, et al. 2003). Atrial- and pacemaker-like cells can also be formed in the differentiating stem cell cultures. The stem cell derived cardiomyocytes are capable of integrating apparently normally when transplanted into rodent and porcine heart muscle, forming gap junction connections between stem cell myocytes and the recipient mouse adult cardiomyocytes (Xue T, et al. 2005; Kehat I, et al. 2004; Hassink R J, et al. 2003).

Type II pneumocytes that express Surfactant Protein C(SPC) (respiratory specific marker) can be generated by coculture of stem cells with mouse embryonic foregut mesenchyme (Denham M, et al. 2002). Stem cells can also be induced to form airway epithelial tissue when differentiated as embryoid bodies or grown on type 1 collagen, and then the resulting Clara cells grown in an air-fluid interface form a pseudostratified surface epithelium (Coraux C, et al. 2005).

Keratinocytes can be derived from stem cells by replating embryoid bodies (Green H, et al. 2003). Cells expressing the transcription factor p63 in the periphery of the secondary cultures identify the keratinocyte progenitors that produce more mature cell types in which cytokeratin 14 and basonuclin are detected. These cells can form terminally differentiated stratifying epithelium but are not the same as keratinocyte epithelium isolated from neonatal or adult skin.

The hematopoietic lineage can be induced to form from differentiating stem cells (Kaufman D S, 2001). Initiating spontaneous differentiation by forming embryoid body cultures and by using a cocktail of hematopoietic cytokines and BMP-4, hematopoietic progenitors that could produce both erythroid and myeloid derivatives can be formed (Chadwick K, et al. 2003). The progenitors are immunologically similar to hematopoietic progenitors of the dorsal aorta. The growth factors that can be used include stem cell factor (SCF), interleukins-3 and -6 (IL-3, IL-6), granulocyte colony-stimulating factor (GCSF) and Flt-3 ligand. A further enhancement of erythroid colonies can be obtained with the addition of vascular endothelial growth factor-A (VEGF-A) (Cerdan C, et al. 2004; Ng E S, et al. 2005). Ng et al. (Ng E S, et al. 2005b) have developed a novel stem cell aggregation system that permits the sequential expression of primitive streak (MLXL1 and Brachyury) and mesoderm markers (Flk1/KDR). Around 1 in 500 stem cells will produce hematopoietic precursors using this system.

Definitive endoderm can be induced in stem cells by restricting culture in serum or by exposure to Activin A (Kubo A, et al. 2004). Some cells of human embryoid bodies will stain positive to insulin antibodies (116), but while they weakly express insulin-2, they do not express insulin-1, do not stain for C-peptide and insulin positive cells are likely to be a result of uptake up of insulin from the culture medium (Rajagopal J, et al. 2003). Some insulin-producing β-like cells can be found in spontaneously differentiating overgrowth conditions of stem cells on MEFs (Brolen G K, et al. 2005).

Insulin producing cells can also be formed from differentiating neuroectoderm (119). Using a modified method of Lumelsky et al. (Lumelsky N, et al. 2001), Segev et al. (Segev H, et al. 2004) have produced islet-like clusters from spontaneously differentiating stem cells. Embryoid bodies were grown for 7 days followed by plating for another week in insulin-transferrin-selenium-fibronectin medium. Disaggregated cultures were allowed to form clusters in medium containing FGF-2 and then exposed to nicotinamide with low glucose in suspension culture. A high percentage of insulin and glucagon or somatostatin coexpressing cells were observed in the cell clusters formed, which were considered to be similar to immature pancreatic cells. Responsiveness to glucose and antagonists was lower than expected and may be due to the immaturity of the pancreatic like cell clusters produced, similar to the poor responsiveness of fetal pancreatic β islet cells.

Rambhatla et al. (2003) reported differentiation of stem cells into cells expressing markers of hepatocytes (albumin, alpha-1-antitrypsin, cytokeratin 8 and 18) and accumulate glycogen, by treatment of differentiating embryoid bodies with sodium butyrate or adherent stem cell cultures with dimethyl sulfoxide followed by sodium butyrate. Others have reported hepatic-like endodermal cells in embryoid bodies (Lavon N, et al. 2004). The selection of cells with particular morphology in adherent stem cell cultures differentiating in vitro can also favor endodermal populations that express markers of fetal liver (Stamp L A, et al. 2003). These data indicate that with the appropriate markers, it will be possible to select cells capable of forming liver, gut and other endodermal tissues.

TABLE 2
Directed Differentiation of stem cells

| Lineage | Primary Inducers | Tissue Type |
|---|---|---|
| Trophectoderm | BMP4 | Trophectoderm |
| Extraembryonic Endoderm | BMP2 | Yolk Sac |
| Germ Cells | NR | Gametes |
| Embryonic Germ Layers | | |
| Ectoderm | Noggin | Neuroectoderm |
| | SDIA + GFs | Midbrain neural |
| | | cells |
| | FGF2-FGF8, SHH | Forebrain and |
| | | midbrain |
| | | TH+ neurons |
| | FGF2 | TH+ neurons |
| | SDIA-BMP4/SHH | Neural crest |
| | FGF-2, EGF, RA | Oligodendrocytes |
| | RA, FGF2-RA, SHH- | Motor neurons |
| | BDNF, GDNF, IGF1 | |
| | p63 expression in EBs | Keratinocytes |
| Mesoderm | END-2 coculture | Cardiomyocytes |
| | BMP4, SCF, IL3, IL6, | Blood |
| | GCSF | |
| | VEGF | Blood |
| | BMP4 or Activin A | Blood |
| Endoderm | Sodium butyrate, DMSO | Hepatocytes |
| | FGF-2, nicotinamide | Pancreatic β cells |

NR—not reported
GFs—growth factors

c) Neural Progenitor Cells

Provided herein are compositions and methods for producing a homogenous population of neural progenitor cells (NPCs) from pluripotent stem cells. Thus, also disclosed is a substantially homogenous population of neural progenitor cells (NPCs) produced using the compositions and methods provided herein. As disclosed herein, NPCs are nestin positive, Oct4 negative, Nanog negative, Sox2 negative, and alkaline phosphatase (AP) negative. NPCs are also oncostatin independent.

Also provided are neurons, astrocytes, and/or oligodendrocytes produced using the compositions and methods provided herein. As disclosed herein, the addition of retinoic acid to nestin-positive progenitors produced by the methods disclosed herein results in reduced expression of the pluripotency marker Oct-4 and increased expression of Pax6. This neural progenitor can then be directed to become either a neuron, astrocyte, or oligodendrocyte. For example, the addition of sonic hedgehog to the Pax6-positive progenitors results in the formation of motor neurons, which are positive for neuronal class III β-Tubulin (TUJ1).

d) Muscle Progenitor Cells

Provided herein are compositions and methods for producing a substantially homogenous population of muscle progenitor cells (myoblasts) from pluripotent stem cells. Thus, also disclosed is a substantially homogenous population of myoblasts produced using the compositions and methods provided herein. Thus, also provided are skeletal, cardiac, and/or smooth muscle cells produced using the compositions and methods provided herein.

As disclosed herein, the addition of forskolin and bromo-cyclin AMP to nestin-positive progenitors produced by the methods disclosed herein results in the formation of a substantially homogenous smooth muscle cells (i.e., stain positive for α-actinin).

7. Single Cell Suspension

An advantage of the herein provided stem cells, such as multipotent stem cells (e.g., adult stem cells, MAPCs, MSCs, and HSCs), pluripotential stem cells (e.g., ES cells, EG cells, PC cells, and EC cells), and OISCs, is the ability to passage the cells in a single cell suspension. As used herein, a single cell suspension refers to a population of cells, wherein at least 20, 30, 40, 50, 60, 70, 80, 90% of the cells are not adhered to any other cell or solid support. Unlike ES cells in the art, the disclosed stem cells can be disaggregated, for example by trypsinization, and replated without substantial loss of cell viability. ES and EG cells remain in clumps or aggregates of cells in order to passage successfully. Thus, in one aspect, the herein disclosed stem cells, such as multipotent stem cells (e.g., adult stem cells, MAPCs, MSCs, and HSCs), pluripotential stem cells (e.g., ES cells, EG cells, PC cells, and EC cells), and OISCs, can be passaged as a cell suspension, wherein at least 20, 30, 40, 50, 60, 70, 80, 90, 95% of the cells are not adhered to any other cell. Thus, provided are compositions of stem cells, such as multipotent stem cells (e.g., adult stem cells, MAPCs, MSCs, and HSCs), pluripotential stem cells (e.g., ES cells, EG cells, PC cells, and EC cells), and OISCs, wherein the cells are in single cell suspension and have the ability to be further cultured without substantial loss of viability. For example at least 50, 60, 70, 80, 90, 95, 96, 97, 98, 99% of the stem cells remain viable when further cultured from single cell suspension.

The ability to passage the cells in a single cell suspension is related to the herein disclosed ability of these cells to differentiate into specific cell types without first generating embryoid bodies. Another advantage of this property of the cells is an increased efficiency in the delivery of compositions such as nucleic acids to the cells. For example, aggregates interfere with the transfection of stem cells inside of an aggregate.

8. Modification of Stem Cells

The herein disclosed stem cells, such as multipotent stem cells (e.g., adult stem cells, MAPCs, MSCs, and HSCs), pluripotential stem cells (e.g., ES cells, EG cells, PC cells, and EC cells), and OISCs, can be genetically modified. A modified stem cell is a stem cell that has a genetic background different than the original background of the cell. For example, a modified stem cell can be a stem cell that expresses a marker from either an extra chromosomal nucleic acid or an integrated nucleic acid. The stem cell can be modified in a number of ways including through the expression of a marker. A marker can be anything that allows for selection or screening of the stem cell or a cell derived from the stem cell. For example, a marker can be a transformation gene, such as Ras, which provides a cell the ability to grow in conditions in which non-transformed cells cannot.

a) Selective Pressure

Cells can be put under a selective pressure which means that the cells are grown or placed under conditions designed to alter the cell population in some way which is related to the marker. For example, if the marker confers antibiotic resistance to the cells that express the marker, then the cell population can be put under conditions where the antibiotic was present. Only cells expressing the gene conveying antibiotic resistance can survive or can have a survival advantage relative to cells not expressing the antibiotic resistance gene. Cells that express the marker gene and have a selective advantage can in some forms of the method be selectively amplified relative to other cells not having the marker meaning they would grow at a rate or survive at a rate greater than the cells not having the marker. In some forms of the method the selection of the cells having the marker has a certain selective stringency. The selective stringency is the efficiency with which the marker identifies cells having the marker from cells that do not have the marker. For example, the selective stringency can be such that the marker producing cells have at least 2, 4, 8, 10, 15, 20, 25, 30, 40, 50, 75, 100, 200, 400, 500, 800, 1000, 2000, 4000, 10,000, 25000, 50,000 fold growth advantage over the non-marker expressing cells. In some forms of the method the selective stringency can be expressed as a selective ratio of the percent of cells expressing the marker that survive over a period of time, for example, a passage, over the percent of cells not expressing the marker that survive over the same time period. For example disclosed are markers that can confer a selective ratio of at least 1, 1.5, 2, 4, 8, 10, 15, 20, 25, 30, 40, 50, 75, 100, 200, 400, 500, 800, 1000, 2000, 4000, 10,000, 25000, 50,000, or 100,000. The markers allow the cells expressing the markers to be selectively grown or visualized which means that the cells expressing the marker can be preferentially or selectively grown or identified over the cells not expressing the marker.

b) Markers

The marker or marker product can used to determine if the marker or some other nucleic acid has been delivered to the cell and once delivered is being expressed. For example, the marker can be the expression product of a marker gene or reporter gene. Examples of useful marker genes include the E. Coli lacZ gene, which encodes β-galactosidase, adenosine phosphoribosyl transferase (APRT), and hypoxanthine phosphoribosyl transferase (HPRT). Fluorescent proteins can also be used as markers and marker products. Examples of fluorescent proteins include green fluorescent protein (GFP), green reef coral fluorescent protein (G-RCFP), cyan fluorescent protein (CFP), red fluorescent protein (RFP or dsRed2) and yellow fluorescent protein (YFP).

(1) Negative Selection Markers

The marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

(2) Dominant Selection Markers

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Other examples include the neomycin analog G418 and puromycin.

(3) Transforming Genes

A transforming gene can be used as a marker. A transforming gene is any sequence that encodes a protein or RNA that causes a cell to have at least one property of a cancer cell, such as the ability to grow in soft agar. Other properties include loss of contact inhibition and independence from growth factors, for example. Also, changes in morphology can occur in transformed cells, such as the cells become less round. Transforming genes can also be referred to as transformation genes. Transforming genes, transformation genes, and their products can be referred to as transforming agents or transformation agents. Transformation agents can also be referred to as immortalization agents.

An oncogene can be a transforming gene and typically a transforming gene will be an oncogene. An oncogene typically codes for a component of a signal transduction cascade. Typically the normal gene product of the oncogene regulates cell growth and a mutation in the protein or expression occurs which deregulates this activity or increases the activity. Oncogenes typically code for molecules in signal transduction pathways, such as the MAPK pathway or Ras pathway, and, for example, can be growth factors, growth factor receptors, transcription factors (erbA: codes a thyroid hormone receptor (steroid receptor), rel: form pairwise combinations that regulate transcription (NF-kB), v-rel: avian reticuloendotheliosis, jun & fos), protein kinases, signal transduction, serine/threonine kinases, nuclear proteins, growth factor receptor kinases, or cytoplasmic tyrosine kinases. It is understood that many oncogenes in combination can become transforming. All sets of combinations of the disclosed oncogenes and transforming genes specifically contemplated. Some oncogenes, such as Ras, are transforming by themselves.

Membrane associated transducing molecules can often be oncogenes. Membrane associated transducing molecules, such as Ras, are indirectly activated by the binding of other molecules to nearby receptors. The activation of the nearby receptors causes the oncogene to become active that starts a signaling cascade which leads to changes in the normal cell behavior. Receptor tyrosine kinases can also be oncogenes. Receptor tyrosine kinases are enzymes that are capable of transferring phosphate groups to target molecules. When a target molecule, such as a growth factor, binds to the extracellular portion of the kinase a signal is transmitted through the cell membrane causing a signal transduction cascade. An example of this type of oncogene is the HER2 protein. Receptor-associated kinases are also membrane associated enzymes but they are activated by binding other nearby receptors. This binding causes the kinase to phosphorylate a target protein causing signal transduction to the nucleus. Src is an example of this type of oncogene. Transcription factors are proteins that bind to specific sequences along the DNA helix causing the bound genes to be expressed in the nucleus. An example of this type of oncogene is myc. Some transcription factors are repressors, such as Rb. Telomerase is a protein-RNA complex that maintains the termini of chromosomes. If telomerase is not present or present in low amounts, chromosomes shorten with each cell division until serious damage occurs. Telomerase is not expressed or present or lowly expressed or present in most normal cells, but is present in concentrations, higher than in a cognate untransformed cell in most transformed cells. Apoptosis regulating proteins are proteins functioning to control programmed cell death. When DNA is damaged or other insults occur, apoptosis can occur. Many oncogenes in their normal state function to block cell death, such as Bcl-2.

A non-limiting list of oncogenes is abl (Tyrosine kinase activity); abl/bcr (New protein created by fusion); Af4/hrx (Fusion effects transcription factor product of hrx); akt-2 (Encodes a protein-serine/threonine kinase Ovarian cancer 1); alk (Encodes a receptor tyrosine kinase); ALK/NPM (New protein created by fusion); aml1 (Encodes a transcription factor); aml1/mtg8 (New protein created by fusion); axl (Encodes a receptor tyrosine kinase); bcl-2, 3, 6 (Block apoptosis (programmed cell death); bcr/abl (New protein created by fusion); c-myc (Cell proliferation and DNA synthesis); dbl (Guanine nucleotide exchange factor); dek/can (New protein created by fusion); E2A/pbx1 (New protein created by fusion); egfr (Tyrosine kinase); enl/hrx (New protein created by fusion); erg/c16 (New protein created by fusion); erbB (Tyrosine kinase); erbB-2 (originally neu) (Tyrosine kinase Breast); ets-1 (Transcription factor for some promoters); ews/fli-1 (New protein created by fusion); fms (Tyrosine kinase); fos (Transcription factor for API); fps (Tyrosine kinase); gip (Membrane associated G protein); gli (Transcription factor); gsp (Membrane associated G protein); HER2/neu (New protein created by gene fusion); hox11 (Over-expression of DNA binding protein); hrx/enl (New protein created by fusion); hrx/af4 (New protein created by fusion); hst (Encodes fibroblast growth factor); IL-3 (Over expression of protein); int-2 (Encodes a fibroblast growth factor); jun (Transcription factor); kit (Tyrosine kinase); KS3 (Growth factor); K-sam (Encodes growth factor receptors); Lbc (Guanine nucleotide exchange factor); ick (Relocation of tyrosine kinase to the T-cell receptor gene); imo-1, (2 Relocation of transcription factor near the T-cell receptor gene); L-myc (Cell proliferation and DNA synthesis); lyl-1 (Over-expression of DNA binding protein); lyt-10 (Relocation of transcription factor near the IgH gene); It-10/C alpha1 (New protein created by fusion); mas (Angiotensin receptor); mdm-2 (Encodes a p53 inhibitor) Sarcomas 1; MLH1 (Mismatch repair in DNA); m11 (New protein created by gene fusion); MLM (Encodes p16 a negative growth regulator that arrests the cell cycle); mos (Serine/threonine kinase); MSH2 (Mismatch repair in DNA); mtg8/aml1 (New protein created by fusion); myb (Encodes a transcription factor with DNA binding domain); MYH11/CBFB (New protein created by fusion); neu (now erb-2) (Tyrosine kinase); N-myc (Cell proliferation and DNA synthesis); NPM/ALK (New protein created by fusion); nrg/rel (New protein created by fusion); ost (Guanine nucleotide axchange factor); pax-5 (Relocation of transcription factor to the IgH gene); pbx1/E2A (New protein created by fusion); pim-1 (Serine/threonine kinase); PML/RAR (New protein created by fusion); PMS1, 2 (Mismatch repair in DNA); PRAD-1 (Encodes cyclin D1 that is important in GI of the cell cycle); raf (Serine/threonine kinase); RAR/PML (New protein created by fusion); rasH (Involved in signal transduction of the cell); rasK (Involved in signal transduction of the cell); rasN (Involved in signal transduction of the cell); rel/nrg (New protein created by fusion); ret (DNA rearrangements that encode a receptor tyrosine kinase); rhom-1, 2 (Over-expression of DNA binding protein); ros (Tyrosine kinase); ski (Transcription factor); sis (Growth factor); set/can (New protein created by gene fusion); Src (Tyrosine kinase); tal-1, 2 (Over-expression of transcription factor); tan-1 (Over-expression of protein); Tiam-1 (Guanine nucleotide exchange factor); TSC2 (GTPase activator); trk (Recombinant fusion protein).

An example of a transforming gene is the Ras gene, an example of which is shown in SEQ ID NO:2. The ras family of oncogenes is comprises 3 main members: -K-ras, H-ras and N-ras. All of three of the oncogenes are involved in a variety of cancers. The K-ras oncogene is found on chromosome 12p12, encoding a 21-kD protein (p21ras). P21 is involved in the G-protein signal transduction pathway. Mutations of the K-ras oncogene produce constitutive activation of the G-protein transduction pathway which results in aberrant proliferation and differentiation.

Activating K-ras mutations are present in greater than 50% of colorectal adenomas and carcinomas, and the vast majority occur at codon 12 of the oncogene. K-ras mutations are one of the most common genetic abnormalities in pancreatic and bile duct carcinomas (greater than 75%). K-ras mutations are also frequent in adenocarcinomas of the lung.

Likewise, the disclosed transforming genes could be paired with other genes or sets of transforming genes that have desirable properties in the particular experiment. Different transformation strategies will be useful in different instances. For example, a cell transformed with an activated/dominant negative pair allows for multiple cycles of reversion. These cells then have the advantages of both primary cells and a cell line. Cells can be expanded, arrested, manipulated, then expanded again. Cells that are reverted using Cre/lox become analogs of primary cells, with only the 34 bp lox site remaining in the genome. These cells could be useful in a cell therapy setting.

c) Expression Systems

The nucleic acids that are delivered to cells typically contain expression controlling systems and often these expression controlling systems are tissues specific. The cells contain an expression controlling system which is tissue specific and possibly another which is not necessarily tissue specific. An expression controlling system is a system which causes expression of a target nucleic acid. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements. Sequences for affecting transcription can be referred to as transcription control elements.

(1) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

The promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

d) Delivery of Compositions to Cells

An advantage of the herein provided stem cells, such as multipotent stem cells (e.g., adult stem cells, MAPCs, MSCs, and HSCs), pluripotential stem cells (e.g., ES cells, EG cells, PC cells, and EC cells), and OISCs, is an increased efficiency in the delivery of compositions such as nucleic acids to the cells. For example, as disclosed herein, at least 40, 45, 50, 55, 60, 70, 80, 81, 82, 83, 84, 85, 86, 87, 88, 89, 90, 91, 92, 93, 94, 95% of the disclosed stem cells, such as multipotent stem cells (e.g., adult stem cells, MAPCs, MSCs, and HSCs), pluripotential stem cells (e.g., ES cells, EG cells, PC cells, and EC cells), and OISCs, in a given culture can be transfected by nucleoporation. This is sharp contrast to the transfection of stem cells in the art that must remain in aggregates to survive, which interferes with the delivery of nucleic acids to the cells inside of an aggregate.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as a Ras expressing nucleic acid, into the cell without degradation and include a promoter yielding expression of the gene in the cells into which it is delivered. The vectors can be derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A viral vector can be used which has been engineered so as to suppress the immune response of the host organism, elicited by the viral antigens. Preferred vectors of this type will carry coding regions for Interleukin 8 or 10.

Viral vectors can have higher transaction abilities (ability to introduce genes) than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, Science 260:926-932 (1993); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Both the E1 and E3 genes can be removed from the adenovirus genome.

(c) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome-19. Vectors which contain this site specific integration property are preferred. An useful form of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(d) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (QIAGEN, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation or nucleoporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described herein, the compositions can be administered in a pharmaceutically acceptable carrier and can be delivered to the subject cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

9. Cells Produced by the Disclosed Methods and Compositions

The adult human body produces many different cell types. Information on human cell types can be found at http://encyclopedia.thefreedictionary.com/List%20of%distinct%20cell%20types%20in%20the%20adult%20human%20body). These different cell types include, but are not limited to, Keratinizing Epithelial Cells, Wet Stratified Barrier Epithelial Cells, Exocrine Secretory Epithelial Cells, Hormone Secreting Cells, Epithelial Absorptive Cells (Gut, Exocrine Glands and Urogenital Tract), Metabolism and Storage cells, Barrier Function Cells (Lung, Gut, Exocrine Glands and Urogenital Tract), Epithelial Cells Lining Closed Internal Body Cavities, Ciliated Cells with Propulsive Function, Extracellular Matrix Secretion Cells, Contractile Cells, Blood and Immune System Cells, Sensory Transducer Cells, Autonomic Neuron Cells, Sense Organ and Peripheral Neuron Supporting Cells, Central Nervous System Neurons and Glial Cells, Lens Cells, Pigment Cells, Germ Cells, and Nurse Cells. Also included are any stem cells and progenitor cells of the cells disclosed herein, as well as the cells they lead to. Cells and cell types of interest produced in the disclosed method can be identified by reference to one or more characteristics of such cells. Many such characteristics are known, some of which are described herein.

a) Cell Types

The usual estimate based on histological studies is that there are ˜200 distinct kinds of cells in an adult human body that show alternate structures and functions (David S. Goodsell, The Machinery of Life, Springer-Verlag, New York, 1993; Bruce Alberts, Dennis Bray, Julian Lewis, Martin Raff, Keith Roberts, James D. Watson, The Molecular Biology of the Cell, Second Edition, Garland Publishing, Inc., New York, 1989; Arthur J. Vander, James H. Sherman, Dorothy S. Luciano, Human Physiology: The Mechanisms of Body Function, Fifth Edition, McGraw-Hill Publishing Company, New York, 1990). These represent discrete categories of cell types of markedly different character, not arbitrary subdivisions along a morphological continuum. Traditional classification is based on microscopic shape and structure, and on crude chemical nature (e.g., affinity for various stains), but newer immunological techniques have revealed, for instance, that there are more than 10 distinct types of lymphocytes. Pharmacological and physiological tests have revealed many different varieties of smooth muscle cells—for example, uterine wall smooth muscle cells are highly sensitive to estrogen and (in late pregnancy) oxytocin, while gut wall smooth muscle cells are not.

Cells of the human body include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting metanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), and Thymus epithelial cell.

This list of cells is organized by cellular function and omits subdivisions of smooth muscle cells, neuron classes in the CNS, various related connective tissue and fibroblast types, and intermediate stages of maturing cells such as keratinocytes (only the stem cell and differentiated cell types are given). Otherwise, the catalog is represents an exhaustive listing of the ˜219 cell varieties found in the adult human phenotype (complexity theory and phylogenetic comparisons suggest that the maximum number of cell types N_{cell}˜N_{gene}^{1/2}=370 cell types for humans with N_{gene}˜10⁵ genes) (S. A. Kauffman, “Metabolic Stability and Epigenesis in Randomly Constructed Genetic Nets,” J. Theoret. Biol. 22(1969):437-467; Stuart A. Kauffman, The Origins of Order: Self-Organization and Selection in Evolution, Oxford University Press, New York, 1993).

b) Cell Markers

There are several identifying characteristics by which a cell can be distinguished and identified. Different cell types are unique in size, shape, density and have distinct expression profiles of intracellular, cell-surface, and secreted proteins. Described are markers that can be used to identify and define a differentiated cell provided herein. These markers can be evaluated using methods known in the art using antibodies, probes, primers, or other such targeting means known in the art. Examples of markers that are routinely used to identify and distinguish differentiated cell types are provided in Table 3.

TABLE 3
Markers Commonly Used to Identify and Characterize Differentiated Cell Types

| | | |
|---|---|---|
| Marker Name | Cell Type | Significance |

Blood Vessel

| | | |
|---|---|---|
| Fetal liver kinase-1 | Endothelial | Cell-surface receptor protein that identifies |
| (Flk1) | | endothelial cell progenitor; marker of cell-cell |
| | | contacts |
| Smooth muscle cell- | Smooth muscle | Identifies smooth muscle cells in the wall of blood |
| specific myosin heavy | | vessels |
| chain | | |
| Vascular endothelial | cadherin | Smooth muscle Identifies smooth muscle cells in |
| cell | | the wall of blood vessels |

Bone

| | | |
|---|---|---|
| Bone-specific alkaline | Osteoblast | Enzyme expressed in osteoblast; activity indicates |
| phosphatase (BAP) | | bone formation |
| Hydroxyapatite | Osteoblast | Minerlized bone matrix that provides structural |
| | | integrity; marker of bone formation |
| Osteocalcin (OC) | Osteoblast | Mineral-binding protein uniquely synthesized by |
| | | osteoblast; marker of bone formation |

Bone Marrow and Blood

| | | |
|---|---|---|
| Bone morphogenetic | Mesenchymal stem | Important for the differentiation of committed |
| protein receptor | and progenitor cells | mesenchymal cell types from mesenchymal stem |
| (BMPR) | | and progenitor cells; BMPR identifies early |
| | | mesenchymal lineages (stem and progenitor cells) |
| CD4 and CD8 | White blood cell | Cell-surface protein markers specific for mature T |
| | (WBC) | lymphocyte (WBC subtype) |
| CD34 | Hematopoietic stem | Cell-surface protein on bone marrow cell, |
| | cell (HSC), satellite, | indicative of a HSC and endothelial progenitor; |
| | endothelial | CD34 also identifies muscle satellite, a muscle |
| | progenitor | stem cell |
| CD34⁺Scal⁺ Lin⁻ | Mesencyhmal stem | Identifies MSCs, which can differentiate into |
| profile | cell (MSC) | adipocyte, osteocyte, chondrocyte, and myocyte |
| CD38 | Absent on HSC | Cell-surface molecule that identifies WBC lineages. |
| | Present on WBC | Selection of CD34⁺/CD38⁻ cells allows for |
| | lineages | purification of HSC populations |
| CD44 | Mesenchymal | A type of cell-adhesion molecule used to identify |
| | | specific types of mesenchymal cells |
| c-Kit | HSC, MSC | Cell-surface receptor on BM cell types that |
| | | identifies HSC and MSC; binding by fetal calf |
| | | serum (FCS) enhances proliferation of ES cells, |
| | | HSCs, MSCs, and hematopoietic progenitor cells |
| Colony-forming unit | HSC, MSC | CFU assay detects the ability of a single stem cell |
| (CFU) | progenitor | or progenitor cell to give rise to one or more cell |
| | | lineages, such as red blood cell (RBC) and/or white |
| | | blood cell (WBC) lineages |
| Fibroblast colony- | Bone marrow | An individual bone marrow cell that has given rise |
| forming unit (CFU-F) | fibroblast | to a colony of multipotent fibroblastic cells; such |
| | | identified cells are precursors of differentiated |
| | | mesenchymal lineages |
| Hoechst dye | Absent on HSC | Fluorescent dye that binds DNA; HSC extrudes the |
| | | dye and stains lightly compared with other cell |
| | | types |
| Leukocyte common | WBC | Cell-surface protein on WBC progenitor |
| antigen (CD45) | | |
| Lineage surface antigen | HSC, MSC | Thirteen to 14 different cell-surface proteins that |
| (Lin) | Differentiated RBC | are markers of mature blood cell lineages; detection |
| | and WBC lineages | of Lin-negative cells assists in the purification of |
| | | HSC and hematopoietic progenitor populations |
| Mac-1 | WBC | Cell-surface protein specific for mature granulocyte |
| | | and macrophage (WBC subtypes) |
| Muc-18 (CD146) | Bone marrow | Cell-surface protein (immunoglobulin superfamily) |
| | fibroblasts, | found on bone marrow fibroblasts, which may be |
| | endothelial | important in hematopoiesis; a subpopulation of |
| | | Muc-18+ cells are mesenchymal precursors |
| Stem cell antigen (Sca- | HSC, MSC | Cell-surface protein on bone marrow (BM) cell, |
| 1) | | indicative of HSC and MSC Bone Marrow and |
| | | Blood cont. |
| Stro-1 antigen | Stromal | Cell-surface glycoprotein on subsets of bone |
| | (mesenchymal) | marrow stromal (mesenchymal) cells; selection of |
| | precursor cells, | Stro-1+ cells assists in isolating mesenchymal |
| | hematopoietic cells | precursor cells, which are multipotent cells that |
| | | give rise to adipocytes, osteocytes, smooth |
| | | myocytes, fibroblasts, chondrocytes, and blood |
| | | cells |
| Thy-1 | HSC, MSC | Cell-surface protein; negative or low detection is |
| | | suggestive of HSC |

Cartilage

| | | |
|---|---|---|
| Collagen types II and | Chondrocyte | Structural proteins produced specifically by |
| IV | | chondrocyte |
| Keratin | Keratinocyte | Principal protein of skin; identifies differentiated |
| | | keratinocyte |
| Sulfated proteoglycan | Chondrocyte | Molecule found in connective tissues; synthesized |
| | | by chondrocyte |

Fat

| | | |
|---|---|---|
| Adipocyte lipid-binding | Adipocyte | Lipid-binding protein located specifically in |
| protein (ALBP) | | adipocyte |
| Fatty acid transporter | Adipocyte | Transport molecule located specifically in |
| (FAT) | | adipocyte |
| Adipocyte lipid-binding | Adipocyte | Lipid-binding protein located specifically in |
| protein (ALBP) | | adipocyte |

Liver

| | | |
|---|---|---|
| Albumin | Hepatocyte | Principal protein produced by the liver; indicates |
| | | functioning of maturing and fully differentiated |
| | | hepatocytes |
| B-1 integrin | Hepatocyte | Cell-adhesion molecule important in cell-cell |
| | | interactions; marker expressed during development |
| | | of liver |

Nervous System

| | | |
|---|---|---|
| CD133 | Neural stem cell, | Cell-surface protein that identifies neural stem |
| | HSC | cells, which give rise to neurons and glial cells |
| Glial fibrillary acidic | Astrocyte | Protein specifically produced by astrocyte |
| protein (GFAP) | | |
| Microtubule-associated | Neuron | Dendrite-specific MAP; protein found specifically |
| protein-2 (MAP-2) | | in dendritic branching of neuron |
| Myelin basic protein | Oligodendrocyte | Protein produced by mature oligodendrocytes;. |
| (MPB) | | located in the myelin sheath surrounding neuronal |
| | | structures |
| Nestin | Neural progenitor | Intermediate filament structural protein expressed |
| | | in primitive neural tissue |
| Neural tubulin | Neuron | Important structural protein for neuron; identifies |
| | | differentiated neuron |
| Neurofilament (NF) | Neuron | Important structural protein for neuron; identifies |
| | | differentiated neuron |
| Noggin | Neuron | A neuron-specific gene expressed during the |
| | | development of neurons |
| O4 | Oligodendrocyte | Cell-surface marker on immature, developing |
| | | oligodendrocyte |
| O1 | Oligodendrocyte | Cell-surface marker that characterizes mature |
| | | oligodendrocyte |
| Synaptophysin | Neuron | Neuronal protein located in synapses; indicates |
| | | connections between neurons |
| Tau | Neuron | Type of MAP; helps maintain structure of the axon |

Pancreas

| | | |
|---|---|---|
| Cytokeratin 19 (CK19) | Pancreatic | CK19 identifies specific pancreatic epithelial cells |
| | epithelium | that are progenitors for islet cells and ductal cells |
| Glucagon | Pancreatic islet | Expressed by alpha-islet cell of pancreas |
| Insulin | Pancreatic islet | Expressed by beta-islet cell of pancreas |
| Pancreas Insulin- | Pancreatic islet | Transcription factor expressed by beta-islet cell of |
| promoting factor-1 | | pancreas |
| (PDX-1) | | |
| Nestin | Pancreatic | Structural filament protein indicative of progenitor |
| | progenitor | cell lines including pancreatic |
| Pancreatic polypeptide | Pancreatic islet | Expressed by gamma-islet cell of pancreas |
| Somatostatin | Pancreatic islet | Expressed by delta-islet cell of pancreas Pluripotent |
| | | Stem Cells |
| Alpha-fetoprotein | Endoderm | Protein expressed during development of primitive |
| (AFP) | | endoderm; reflects endodermal differentiation |

Pluripotent Stem Cells

| | | |
|---|---|---|
| Bone morphogenetic | Mesoderm | Growth and differentiation factor expressed during |
| protein-4 | | early mesoderm formation and differentiation |
| Brachyury | Mesoderm | Transcription factor important in the earliest phases |
| | | of mesoderm formation and differentiation; used as |
| | | the earliest indicator of mesoderm formation |
| GATA-4 gene | Endoderm | Expression increases as ES differentiates into |
| | | endoderm |
| Hepatocyte nuclear | Endoderm | Transcription factor expressed early in endoderm |
| factor-4 (HNF-4) | | formation |
| Nestin | Ectoderm, neural | Intermediate filaments within cells; characteristic |
| | and pancreatic | of primitive neuroectoderm formation |
| | progenitor | |
| Neuronal cell-adhesion | Ectoderm | Cell-surface molecule that promotes cell-cell |
| molecule (N-CAM) | | interaction; indicates primitive neuroectoderm |
| | | formation |
| Pax6 | Ectoderm | Transcription factor expressed as ES cell |
| | | differentiates into neuroepithelium |
| Vimentin | Ectoderm, neural | Intermediate filaments within cells; characteristic |
| | and pancreatic | of primitive neuroectoderm formation |
| | progenitor | |

Skeletal Muscle/Cardiac/Smooth Muscle

| | | |
|---|---|---|
| MyoD and Pax7 | Myoblast, myocyte | Transcription factors that direct differentiation of |
| | | myoblasts into mature myocytes |
| Myogenin and MR4 | Skeletal myocyte | Secondary transcription factors required for |
| | | differentiation of myoblasts from muscle stem cells |
| Myosin heavy chain | Cardiomyocyte | A component of structural and contractile protein |
| | | found in cardiomyocyte |
| Myosin light chain | Skeletal myocyte | A component of structural and contractile protein |
| | | found in skeletal myocyte |

Cell surface antigens are routinely used as markers to identify and distinguish cells. Antigenic specificities exist for species (xenotype), organ, tissue, or cell type for almost all cells—possibly involving as many as ˜10⁴ distinct antigens. Examples of cell surface antigens that can be used to distinguish cell types are provided in Table 4.

TABLE 4
Human Cell Surface Antigens

| | |
|---|---|
| B cell | CD1C, CHST10, HLA-A, HLA-DRA, NT5E |
| Activated B Cells | CD28, CD38, CD69, CD80, CD83, CD86, DPP4, FCER2, IL2RA, |
| | TNFRSF8, TNFSF7 |
| Mature B Cells | CD19, CD22, CD24, CD37, CD40, CD72, CD74, CD79A, CD79B, |
| | CR2, IL1R2, ITGA2, ITGA3, MS4A1, ST6GAL1 |
| T cell | CD160, CD28, CD37, CD3D, CD3G, CD3Z, CD5, CD6, CD7, FAS, |
| | KLRB1, KLRD1, NT5E, ST6GAL1 |
| Cytotoxic T Cells | CD8A, CD8B1 |
| Helper T Cells | CD4 |
| Activated T Cells | ALCAM, CD2, CD38, CD40LG, CD69, CD83, CD96, CTLA4, DPP4, |
| | HLA-DRA, IL12RB1, IL2RA, ITGA1, TNFRSF4, TNFRSF8, |
| | TNFSF7 |
| Natural Killer (NK) | CD2, CD244, CD3Z, CD7, CD96, CHST10, FCGR3B, IL12RB1, |
| cell | KLRB1, KLRC1, KLRD1, LAG3, NCAM1 |
| Monocyte/macrophage | ADAM8, C5R1, CD14, CD163, CD33, CD40, CD63, CD68, CD74, |
| | CD86, CHIT1, CHST10, CSF1R, DPP4, FABP4, FCGR1A, HLA- |
| | DRA, ICAM2, IL1R2, ITGA1, ITGA2, S100A8, TNFRSF8, TNFSF7 |
| Activated | CD69, ENG, FCER2, IL2RA |
| Macrophages | |
| Endothelial cell | ACE, CD14, CD34, CD31, CDH5, ENG, ICAM2, MCAM, NOS3, |
| | PECAM1, PROCR, SELE, SELP, TEK, THBD, VCAM1, VWF. |
| Smooth muscle cell | ACTA2, MYH10, MYH11, MYH9, MYOCD. |
| Dendritic cell | CD1A, CD209, CD40, CD83, CD86, CR2, FCER2, FSCN1 |
| Mast cell | C5R1, CMA1, FCER1A, FCER2, TPSAB1 |
| Fibroblast (stromal) | ALCAM, CD34, COL1A1, COL1A2, COL3A1, PH-4 |
| Epithelial cell | CD1D, K6IRS2, KRT10, KRT13, KRT17, KRT18, KRT19, KRT4, |
| | KRT5, KRT8, MUC1, TACSTD1. |
| Adipocyte | ADIPOQ, FABP4, RETN. |

In the case of red blood cells, antigens in the Rh, Kell, Duffy, and Kidd blood group systems are found exclusively on the plasma membranes of erythrocytes and have not been detected on platelets, lymphocytes, granulocytes, in plasma, or in other body secretions such as saliva, milk, or amniotic fluid (P. L. Mollison, C. P. Engelfriet, M. Contreras, Blood Transfusions in Clinical Medicine, Ninth Edition, Blackwell Scientific, Oxford, 1993). Thus detection of any member of this four-antigen set establishes a unique marker for red cell identification. MNSs and Lutheran antigens are also limited to erythrocytes with two exceptions: GPA glycoprotein (MN activity) also found on renal capillary endothelium (P. Hawkins, S. E. Anderson, J. L. McKenzie, K. McLoughlin, M. E. J. Beard, D. N. J. Hart, “Localization of MN Blood Group Antigens in Kidney,” Transplant. Proc. 17(1985):1697-1700), and Lu^{b}-like glycoprotein which appears on kidney endothelial cells and liver hepatocytes (D. J. Anstee, G. Mallinson, J. E. Yendle, et al., “Evidence for the occurrence of Lub-active glycoproteins in human erythrocytes, kidney, and liver,” International Congress ISBT-BBTS Book of Abstracts, 1988, p. 263). In contrast, ABH antigens are found on many non-RBC tissue cells such as kidney and salivary glands (Ivan M. Roitt, Jonathan Brostoff, David K. Male, Immunology, Gower Medical Publishing, New York, 1989). In young embryos ABH can be found on all endothelial and epithelial cells except those of the central nervous system (Aron E. Szulman, “The ABH antigens in human tissues and secretions during embryonal development,” J. Histochem. Cytochem. 13(1965):752-754). ABH, Lewis, I and P blood group antigens are found on platelets and lymphocytes, at least in part due to adsorption from the plasma onto the cell membrane. Granulocytes have I antigen but no ABH (P. L. Mollison, C. P. Engelfriet, M. Contreras, Blood Transfusions in Clinical Medicine, Ninth Edition, Blackwell Scientific, Oxford, 1993).

Platelets also express platelet-specific alloantigens on their plasma membranes, in addition to the HLA antigens they already share with body tissue cells. Currently there are five recognized human platelet alloantigen (HPA) systems that have been defined at the molecular level. The phenotype frequencies given are for the Caucasian population; frequencies in African and Asian populations may vary substantially. For instance, HPA-1b is expressed on the platelets of 28% of Caucasians but only 4% of the Japanese population (Thomas J. Kunicki, Peter J. Newman, “The molecular immunology of human platelet proteins,” Blood 80(1992):1386-1404).

Lymphocytes with a particular functional activity can be distinguished by various differentiation markers displayed on their cell surfaces. For example, all mature T cells express a set of polypeptide chains called the CD3 complex. Helper T cells also express, the CD4 glycoprotein, whereas cytotoxic and suppressor T cells express a marker called CD8 (Wayne M. Becker, David W. Deamer, The World of the Cell, Second Edition, Benjamin/Cummings. Publishing Company, Redwood City Calif., 1991). Thus the phenotype CD3⁺ CD4⁺CD8⁻ positively identifies a helper T cell, whereas the detection of CD3⁺ CD4⁻CD8⁺ uniquely identifies a cytotoxic or suppressor T cell. All B lymphocytes express immunoglobulins (their antigen receptors, or Ig) on their surface and can be distinguished from T cells on that basis, e.g., as Ig⁺ MHC Class II⁺.

Lymphocyte surfaces also display distinct markers representing specific gene products that are expressed only at characteristic stages of cell differentiation. For example, Stage I Progenitor B cells display CD34⁺PhiL⁻CD19⁻; Stage II, CD34⁺PhiL⁺CD19⁻; Stage III, CD34⁺PhiL⁺CD19⁺; and finally CD34⁻PhiL⁺CD19⁺ at the Precursor B stage (Una Chen, “Chapter 33. Lymphocyte Engineering, Its Status of Art and Its Future,” in Robert P. Lanza, Robert Langer, William L. Chick, eds., Principles of Tissue Engineering, R. G. Landes Company, Georgetown Tex., 1997, pp. 527-561).

There are neutrophil-specific antigens and various receptor-specific immunoglobulin binding specificities for leukocytes. For instance, monocyte FcRI receptors display the measured binding specificity IgG1⁺⁺⁺IgG2⁻IgG3⁺⁺⁺IgG4⁺, monocyte FcRIII receptors have IgG⁺⁺IgG2⁻IgG3⁺⁺IgG4⁻, and FcRII receptors on neutrophils and eosinophils show IgG1⁺⁺⁺IgG2⁺IgG3⁺⁺⁺IgG4⁺. Neutrophils also have β-glucan receptors on their surfaces (Vicki Glaser, “Carbohydrate-Based Drugs Move CLoser to Market,” Genetic Engineering News, 15 Apr. 1998, pp. 1, 12, 32, 34).

Tissue cells display specific sets of distinguishing markers on their surfaces as well. Thyroid microsomal-microvillous antigen is unique to the thyroid gland (Ivan M. Roitt, Jonathan Brostoff, David K. Male, Immunology, Gower Medical Publishing, New York, 1989). Glial fibrillary acidic protein (GFAP) is an immunocytochemical marker of astrocytes (Carlos Lois, Jose-Manuel Garcia-Verdugo, Arturo Alvarez-Buylla, “Chain Migration of Neuronal Precursors,” Science 271(16 Feb. 1996):978-981), and syntaxin 1A and 1B are phosphoproteins found only in the plasma membrane of neuronal cells (Nicole Calakos, Mark K. Bennett, Karen E. Peterson, Richard H. Scheller, “Protein-Protein Interactions Contributing to the Specificity of Intracellular Vesicular Trafficking,” Science 263(25 Feb. 1994): 1146-1149). Alpha-fodrin is an organ-specific autoantigenic marker of salivary gland cells (Norio Haneji, Takanori Nakamura, Koji Takio, et al., “Identification of alpha-Fodrin as a Candidate Autoantigen in Primary Sjogren's Syndrome,” Science 276(25 Apr. 1997):604-607). Fertilin, a member of the ADAM family, is found on the plasma membrane of mammalian sperm cells (Tomas Martin, Ulrike Obst, Julius Rebek Jr., “Molecular Assembly and Encapsulation Directed by Hydrogen-Bonding Preferences and the Filling of Space,” Science 281(18 Sep. 1998):1842-1845). Hepatocytes display the phenotypic markers ALB⁺⁺⁺GGT⁻CK19⁻ along with connexin 32, transferrin, and major urinary protein (MUP), while biliary cells display the markers AFP⁻GGT⁺⁺⁺CK19⁺⁺⁺ plus BD.1 antigen, alkaline phosphatase, and DPP4 (Lola M. Reid, “Chapter 31. Stem Cell/Lineage Biology and Lineage-Dependent Extracellular Matrix Chemistry: Keys to Tissue Engineering of Quiescent Tissues such as Liver,” in Robert P. Lanza, Robert Langer, William L. Chick, eds., Principles of Tissue Engineering, R. G. Landes Company, Georgetown Tex., 1997, pp. 481-514). A family of 100-kilodalton plasma membrane guanosine triphosphatases implicated in clathrin-coated vesicle transport include dynamin I (expressed exclusively in neurons), dynamin II (found in all tissues), and dynamin III (restricted to the testes, brain, and lungs), each with at least four distinct isoforms; dynamin II also exhibits intracellular localization in the trans-Golgi network (Martin Schnorf, Ingo Potrykus, Gunther Neuhaus, “Microinjection Technique: Routine System for Characterization of Microcapillaries by Bubble Pressure Measurement,” Experimental Cell Research 210(1994):260-267). Table 5 lists numerous unique antigenic markers of hepatopoietic (e.g., hepatoblast) and hemopoietic (e.g., erythroid progenitor) cells.

TABLE 5
Unique antigenic markers of hepatopoietic and hemopoietic human cells.

| | |
|---|---|
| Hepatopoietic Cells | α-fetoprotein, albumin, stem cell factor, hepatic heparin sulfate-PGs |
| (e.g., Hepatoblasts) | (syndecan/perlecans), IGF I, IGF II, TGF-α, TGF-α receptor, α1 |
| | integrin, α5 integrin, connexin 26, and connexin 32 |
| Hematopoietic Cells | OX43 (MCA 276), OX44 (MCA 371, CD37), OX42 (MCA 275, |
| (e.g., Erythroid | CD118), c-Kit, stem cell factor receptor, hemopoietic heparin sulfate- |
| Progenitors) | PG (serglycin), GM-CSF, CSF, α4 integrin, and red blood cell antigen |

At least four major families of cell-specific cell adhesion molecules had been identified by 1998—the immunoglobulin (Ig) superfamily (including N-CAM and ICAM-1), the integrin superfamily, the cadherin family and the selectin family (see below).

Integrins are ˜200 kilodalton cell surface adhesion receptors expressed on a wide variety of cells, with most cells expressing several integrins. Most integrins, which mediate cellular connection to the extracellular matrix, are involved in attachments to the cytoskeletal substratum. Cell-type-specific examples include platelet-specific integrin (α_{IIb}β₃), leukocyte-specific β₂ integrins, late-activation (α_{L}β₂) lymphocyte antigens, retinal ganglion axon integrin (α₆β₁) and keratinocyte integrin (α₅β₁) (Richard O. Hynes, “Integrins: Versatility, Modulation, and Signaling in Cell Adhesion,” Cell 69(3 Apr. 1992): 11-25). At least 20 different heterodimer integrin receptors were known in 1998.

The cadherin molecular family of 723-748-residue transmembrane proteins provides yet another avenue of cell-cell adhesion that is cell-specific (Masatoshi Takeichi, “Cadherins: A molecular family important in selective cell-cell adhesion,” Ann. Rev. Biochem. 59(1990):237-252). Cadherins are linked to the cytoskeleton. The classical cadherins include E-(epithelial), N-(neural or A-CAM), and P— (placental) cadherin, but in 1998 at least 12 different members of the family were known (Elizabeth J. Luna, Anne L. Hitt, “Cytoskeleton-Plasma Membrane Interactions,” Science 258 (1992):955-964). They are concentrated (though not exclusively found) at cell-cell junctions on the cell surface and appear to be crucial for maintaining multicellular architecture. Cells adhere preferentially to other cells that express the identical cadherin type. Liver hepatocytes express only E-; mesenchymal lung cells, optic axons and neuroepithelial cells express only N-; epithelial lung cells express both E- and P-cadherins. Members of the cadherin family also are distributed in different spatiotemporal patterns in embryos, with the expression of cadherin types changing dynamically as the cells differentiate (Masatoshi Takeichi, “Cadherins: A molecular family important in selective cell-cell adhesion,” Ann. Rev. Biochem. 59(1990):237-252).

Carbohydrates are crucial in cell recognition. All cells have a thin sugar coating (the glycocalyx) consisting of glycoproteins and glycolipids, of which ˜3000 different motifs had been identified by 1998. The repertoire of carbohydrate cell surface structures changes characteristically as the cell develops, differentiates, or sickens. For example, a unique trisaccharide (SSEA-1 or Lex) appears on the surfaces of cells of the developing embryo exactly at the 8- to 16-cell stage when the embryo compacts from a group of loose cells into a smooth ball.

Carbohydrate motifs are in theory more combinatorially diverse than nucleotide or protein-based structures. While nucleotides and amino acids can interconnect in only one way, the monosaccharide units in oligosaccharides and polysaccharides can attach at multiple points. Thus two amino acids can make only two distinct dipeptides, but two identical monosaccharides can bond to form 11 different disaccharides because each monosaccharide has 6 carbons, giving each unit 6 different attachment points for a total of 6+5=11 possible combinations. Four different nucleotides can make only 24 distinct tetranucleotides, but four different monosaccharides can make 35,560 unique tetrasaccharides, including many with branching structures (Nathan Sharon, Halina Lis, “Carbohydrates in Cell Recognition,” Scientific American 268(January 1993):82-89). A single hexasaccharide can make ˜10¹² distinct structures, vs. only 6.4×10⁷ structures for a hexapeptide; a 9-mer carbohydrate has a mole of isomers (Roger A. Laine. Glycobiology 4(1994):1-9).

The CD44 family of transmembrane glycoproteins are 80-95 kilodalton cell adhesion receptors that mediate ECM binding, cell migration and lymphocyte homing. CD44 antigen shows a wide variety of cell-specific and tissue-specific glycosylation patterns, with each cell type decorating the CD44 core protein with its own unique array of carbohydrate structures (Jayne Lesley, Robert Hyman, Paul W. Kincade, “CD44 and Its Interaction with Extracellular Matrix,” Advances in Immunology 54(1993):271-335; Tod A. Brown, Todd Bouchard, Tom St. John, Elizabeth Wayner, William G. Carter, “Human Keratinocytes Express a New CD44 Core Protein (CD44E) as a Heparin-Sulfate Intrinsic Membrane Proteoglycan with Additional Exons,” J. Cell Biology 113(April 1991):207-221). Distinct CD44 cell surface molecules have been found in lymphocytes, macrophages, fibroblasts, epithelial cells, and keratinocytes. CD44 expression in the nervous system is restricted to the white matter (including astrocytes and glial cells) in healthy young people, but appears in gray matter accompanying age or disease (Jayne Lesley, Robert Hyman, Paul W. Kincade, “CD44 and Its Interaction with Extracellular Matrix,” Advances in Immunology 54(1993):271-335). A few tissues are CD44 negative, including liver hepatocytes, kidney tubular epithelium, cardiac muscle, the testes, and portions of the skin.

The selectin family of ˜50 kilodalton cell adhesion receptor glycoprotein molecules (Ajit Varki, “Selectin ligands,” Proc. Natl. Acad. Sci. USA 91(August 1994):7390-7397; Masatoshi Takeichi, “Cadherins: A molecular family important in selective cell-cell adhesion,” Ann. Rev. Biochem. 59(1990):237-252) can recognize diverse cell-surface antigen carbohydrates and help localize leukocytes to regions of inflammation (leukocyte trafficking). Selectins are not attached to the cytoskeleton (Elizabeth J. Luna, Anne L. Hitt, “Cytoskeleton-Plasma Membrane Interactions,” Science 258(6 Nov. 1992):955-964). Leukocytes display L-selectin, platelets display P-selectin, and endothelial cells display E-selectin (as well as L and P) receptors. Cell-specific molecules recognized by selectins include tumor mucin oligosaccharides (recognized by L, P, and E), brain glycolipids (P and L), neutrophil glycoproteins (E and P), leukocyte sialoglycoproteins (E and P), and endothelial proteoglycans (P and L) (Ajit Varki, (1994). The related MEL-14 glycoprotein homing receptor family allows lymphocyte homing to specific lymphatic tissues coded with “vascular addressin”—cell-specific surface antigens found on cells in the intestinal Peyer's patches, the mesenteric lymph nodes, lung-associated lymph nodes, synovial cells and lactating breast endothelium. Homing receptors also allow some lymphocytes to distinguish between colon and jejunum (Ted A. Yednock, Steven D. Rosen, “Lymphocyte Homing,” Advances in Immunology 44(1989):313-378; Lloyd M. Stoolman, “Adhesion Molecules Controlling Lymphocyte Migration,” Cell 56(24 Mar. 1989):907-910). Selectin-related interactions, along with chemoattractant receptors and with integrin-Ig, regulate leukocyte extravasation in series, establishing a three-digit “area code” for cell localization in the body (Timothy A. Springer, “Traffic Signals on Endothelium for Lymphocyte Recirculation and Leukocyte Emigration,” Annu. Rev. Physiol. 57(1995):827-872).

Finally, cells may be typed according to their indigenous transmembrane cytoskeleton-related proteins. For example, erythrocyte membranes contain glycophorin C (˜25 kilodaltons, ˜3000 molecules/micron²) and band 3 ion exchanger (90-100 kilodaltons, ˜10,000 molecules/micron²) (Elizabeth J. Luna, Anne L. Hitt, “Cytoskeleton-Plasma Membrane Interactions,” Science 258(6 Nov. 1992):955-964; M. J. Tanner, “The major integral proteins of the human red cell,” Baillieres Clin. Haematol. 6(June 1993):333-356); platelet membranes incorporate the GP Ib-IX glycoprotein complex (186 kilodaltons); cell membrane extensions in neutrophils require the transmembrane protein ponticulin (17 kilodaltons); and striated muscle cell membranes contain a specific laminin-binding glycoprotein (156 kilodaltons) at the outermost part of the transmembrane dystrophin-glycoprotein complex (Elizabeth J. Luna, Anne L. Hitt, “Cytoskeleton-Plasma Membrane Interactions,” Science 258(6 Nov. 1992):955-964). There are also a variety of carbohydrate-binding proteins (lectins) that appear frequently on cell surfaces, and can distinguish different monosaccharides and oligosaccharides (Nathan Sharon, Halina Lis, “Carbohydrates in Cell Recognition,” Scientific American 268(January 1993):82-89). Cell-specific lectins include the galactose (asialoglycoprotein)-binding and fucose-binding lectins of hepatocytes, the mannosyl-6-phosphate (M6P) lectin of fibroblasts, the mannosyl-N-acetylglucosamine-binding lectin of alveolar macrophages, the galabiose-binding lectins of uroepithelial cells, and several galactose-binding lectins in heart, brain and lung (Nathan Sharon, (1993); Mark J. Poznansky, Rudolph L. Juliano, “Biological Approaches to the Controlled Delivery of Drugs: A Critical Review,” Pharmacological Reviews 36(1984):277-336; Karl-Anders Karlsson, “Glycobiology: A Growing Field for Drug Design,” Trends in Pharmacological Sciences 12(July 1991):265-272; N. Sharon, H. Lis, “Lectins—proteins with a sweet tooth: functions in cell recognition,” Essays Biochem. 30(1995):59-75).

Further description of cell types that can be produced in the disclosed method is provided below and elsewhere herein.

c) Keratinizing Epithelial Cells

Keratinizing Epithelial Cells include which includes Epidermal keratinocytes ((differentiating epidermal cell)). The keratinocyte makes up approximately 90% of the cells of the epidermis. The epidermis is divided into four layers based on keratinocyte morphology: which includes the basal layer (at the junction with the dermis), the stratum granulosum, the stratum spinosum, and the stratum corneum. Keratinocytes begin their development in the basal layer through keratinocyte stem cell differentiation. They are pushed up through the layers of the epidermis, undergoing gradual differentiation until they reach the stratum corneum where they form a layer of dead, flattened, highly keratinised cells called squames. This layer forms an effective barrier to the entry of foreign matter and infectious agents into the body and minimizes moisture loss. Keratinizing Epithelial Cells also include Epidermal basal cells which are epidermal stem cells. Keratinizing Epithelial Cells also include Keratinocytes of fingernails and toenails, Nail bed basal cells (a stem cell), Medullary hair shaft cells, Cortical hair shaft cells, Cuticular hair shaft cells, Cuticular hair root sheath cells, Hair root sheath cells of Huxley's layer, Hair root sheath cells of Henle's layer, External hair root sheath cells, and Hair matrix cells (a stem cell). Also included are any stem cells and progenitor cells of the cells disclosed herein, as well as the cells they lead to.

d) Wet Stratified Barrier Epithelial Cells

The human Wet Stratified Barrier Epithelial Cells include surface epithelial cells of the stratified squamous epithelium of the cornea, tongue, oral cavity, esophagus, anal canal, distal urethra, and vagina, as well as basal cells (stem cells) of the epithelia of cornea, tongue, oral cavity, esophagus, anal canal, distal urethra and vagina, and urinary epithelium cells (lining the bladder and urinary tracks. Also included are any stem cells and progenitor cells of the cells disclosed herein, as well as the cells they lead to.

In zootomy, epithelium is a tissue composed of epithelial cells. Such tissue typically covers parts of the body, like a cell membrane covers a cell. It is also used to form glands. The outermost layer of human skin and mucous membranes of mouths and body cavities are made up of dead squamous epithelial cells. Epithelial cells also line the insides of the lungs, the gastrointestinal tract, the reproductive and urinary tracts, and make up the exocrine and endocrine glands. Also included are any stem cells and progenitor cells of the cells disclosed herein, as well as the cells they lead to.

e) Exocrine Secretory Epithelial Cells

Exocrine secretory epithelial cells include Salivary gland mucous cells (which produce polysaccharide-rich secretions), Salivary gland serous cell (glycoprotein-enzyme rich secretion), Von Ebner's gland cell in tongue (washes taste buds), Mammary gland cells (milk secretion), Lacrimal gland cell (tear secretion), and Ceruminous gland cell in ear (wax secretion), Eccrine sweat gland dark cells, (Glycoprotein secretion) Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose, Brunner's gland cell in duodenum (enzymes and alkaline mucus), Seminal vesicle cell (secretes seminal fluid components), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), and Clara cell of lung. Also included are any stem cells and progenitor cells of the cells disclosed herein, as well as the cells they lead to.

f) Hormone Secreting Cells

Hormone secreting cells include Anterior pituitary cells, Somatotropes, Lactotropes, Thyrotropes, Gonadotropes, Corticotropes, Intermediate pituitary cell, secreting melanocyte-stimulating hormone, Magnocellular neurosecretory cells, secreting oxytocin, secreting vasopressin, Gut and respiratory tract cells secreting serotonin, secreting endorphin, secreting somatostatin, secreting gastrin, secreting secretin, secreting cholecystokinin, secreting insulin, secreting glucagon, secreting bombesin, Thyroid gland cells, thyroid epithelial cell, parafollicular cell, Parathyroid gland cells, Parathyroid chief cell, oxyphil cell, Adrenal gland cells, chromaffin cells, secreting steroid hormones (mineralcorticoids and glucocorticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, and Mesangial cell of kidney. Also included are any stem cells and progenitor cells of the cells disclosed herein, as well as the cells they lead to.

g) Epithelial Absorptive Cells (Gut, Exocrine Glands and Urogenital Tract)

Epithelial Absorptive Cells include, Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, and Epididymal basal cell. Also included are any stem cells and progenitor cells of the cells disclosed herein, as well as the cells they lead to:

h) Metabolism and Storage Cells

Metabolism and Storage cells include, Hepatocyte (liver cell), White fat cell, Brown fat cell, and Liver lipocyte. Also included are any stem cells and progenitor cells of the cells disclosed herein, as well as the cells they lead to.

i) Barrier Function Cells (Lung, Gut, Exocrine Glands and Urogenital Tract)

Barrier Function Cells include Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, and Duct cell (of seminal vesicle, prostate gland, etc.). Also included are any stem cells and progenitor cells of the cells disclosed herein, as well as the cells they lead to.

j) Epithelial Cells Lining Closed Internal Body Cavities

Epithelial Cells Lining Closed Internal Body Cavities include Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, and Corneal endothelial cell. Also included are any stem cells and progenitor cells of the cells disclosed herein, as well as the cells they lead to.

k) Ciliated Cells with Propulsive Function

Ciliated Cells with Propulsive Function include, Respiratory tract ciliated cell, Oviduct ciliated cell (in female), Uterine endometrial ciliated cell (in female), Rete testis cilated cell (in male), Ductulus efferens ciliated cell (in male), and Ciliated ependymal cell of central nervous system (lining brain cavities). Also included are any stem cells and progenitor cells of the cells disclosed herein, as well as the cells they lead to.

l) Extracellular Matrix Secretion Cells

Extracellular Matrix Secretion Cells include Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other nonepithelial fibroblasts, Blood capillary pericyte, Nucleus, pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, and Stellate cell of perilymphatic space of ear. Also included are any stem cells and progenitor cells of the cells disclosed herein, as well as the cells they lead to.

m) Contractile Cells

Contractile Cells include Red skeletal muscle cell (slow), White skeletal muscle cell (fast), Intermediate skeletal muscle cell, nuclear bag cell of Muscle spindle, nuclear chain cell of Muscle spindle, Satellite cell (stem cell), Ordinary heart muscle cell, Nodal heart muscle cell, Purkinje fiber cell, Smooth muscle cell (various types), Myoepithelial cell of iris, and Myoepithelial cell of exocrine glands. Also included are any stem cells and progenitor cells of the cells disclosed herein, as well as the cells they lead to.

n) Blood and Immune System Cells

Blood and Immune System Cells include, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, B cells, Natural killer cell, Reticulocyte, and Stem cells and committed progenitors for the blood and immune system (various types). Also included are any stem cells and progenitor cells of the cells disclosed herein, as well as the cells they lead to.

o) Sensory Transducer Cells

Sensory Transducer Cells include Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory receptor neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), and Proprioceptive primary sensory neurons (various types). Also included are any stem cells and progenitor cells of the cells disclosed herein, as well as the cells they lead to.

p) Autonomic Neuron Cells

Autonomic Neuron Cells include Cholinergic neural cell (various types), Adrenergic neural cell (various types), and Peptidergic neural cell (various types). Also included are any stem cells and progenitor cells of the cells disclosed herein, as well as the cells they lead to.

q) Sense Organ and Peripheral Neuron Supporting Cells

Sense Organ and Peripheral Neuron Supporting Cells include Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), and Enteric glial cell. Also included are any stem cells and progenitor cells of the cells disclosed herein, as well as the cells they lead to.

r) Central Nervous System Neurons and Glial Cells

Central Nervous System Neurons and Glial Cells include Neuron cells (large variety of types), Astrocyte glial cell (various types), and Oligodendrocyte glial cell. Also included are any stem cells and progenitor cells of the cells disclosed herein, as well as the cells they lead to.

s) Lens Cells

Lens Cells include Anterior lens epithelial cell, and Crystallin-containing lens fiber cell. Also included are any stem cells and progenitor cells of the cells disclosed herein, as well as the cells they lead to.

t) Pigment Cell

Pigment Cells include Melanocyte and Retinal pigmented epithelial cell. Also included are any stem cells and progenitor cells of the cells disclosed herein, as well as the cells they lead to.

u) Germ Cells

Germ Cells include Oogonium/oocyte, Spermatocyte, and Spermatogonium cell (stem cell for spermatocyte). Also included are any stem cells and progenitor cells of the cells disclosed herein, as well as the cells they lead to.

v) Nurse Cells

Nurse Cells include Ovarian follicle cell, Sertoli cell (in testis), and Thymus epithelial cell. Also included are any stem cells and progenitor cells of the cells disclosed herein, as well as the cells they lead to.

The disclosed stem cells can be differentiated into cell types described above.

10. Characteristics and Techniques for Compositions and Methods

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that can arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences can be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions can be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example, Ras, as well as any other proteins disclosed herein, as well as various functional nucleic acids. The disclosed nucleic acids are made up of, for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to, for example, Ras, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and Probes

Disclosed are compositions including primers and probes, which are capable of interacting with the genes disclosed herein. The primers can be used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. The primers can be used for the DNA amplification reactions, such as PCR or direct sequencing. It is understood that the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

(4) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules. It is also understood that vectors expressing functional nucleic acids can be transfected into the disclosed stem cells.

Functional nucleic acid molecules can interact with any macromolecule, such as DNA, RNA, polypeptides, or carbohydrate chains, or cells. Thus, functional nucleic acids can interact with the disclosed stem cells. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule can be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of United States patents: U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of Ras aptamers, the background protein could be Serum albumin. Representative examples of how to make and use aptamers to bind a variety of different target molecules can be found in the following non-limiting list of United States patents: U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following United States patents: U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following United States patents: U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following United States patents: U.S. Pat. Nos. 5,595,873 and 5,652,107). There are also a number of ribozymes that are not found in natural systems, but which have been engineered to catalyze specific reactions de novo (for example, but not limited to the following United States patents: U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of United States patents: U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of United States patents: U.S. Pat. Nos. 5,176,996, 5,645,985, 5,650,316, 5,683,874, 5,693,773, 5,834,185, 5,869,246, 5,874,566, and 5,962,426.

External guide sequences (EGSS) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of United States patents: U.S. Pat. Nos. 5,168,053, 5,624,824, 5,683,873, 5,728,521, 5,869,248, and 5,877,162.

It is also understood that the disclosed nucleic acids can be used for RNAi or RNA interference. It is thought that RNAi involves a two-step mechanism for RNA interference (RNAi): an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that can act in RNAi. For description of making and using RNAi molecules see See, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least form material related to delivery and making of RNAi molecules.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

d) Peptides

(1) Protein Variants

There are numerous variants of the disclosed proteins that are known and herein contemplated. In addition, to the known functional strain variants there are derivatives of the proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 6 and 7 and are referred to as conservative substitutions.

TABLE 6
Amino Acid Abbreviations

| | |
|---|---|
| Amino Acid | Abbreviations |

| | | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 7
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 7, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues can be deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular cell from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 6 and Table 7. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations (Rizo and Gierasch, Ann. Rev. Biochem. 61:387 (1992), incorporated herein by reference).

e) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone can range from about 1 μg/kg to up to 100 mg/kg of body weight or more per day, depending on the factors mentioned above.

f) Chips and Microarrays

Disclosed are chips where at least one address is the sequences or part of the sequences set forth in any of the nucleic acid sequences, peptides, or cells disclosed herein. Also disclosed are chips where at least one address is the sequences or portion of sequences set forth in any of the peptide sequences disclosed herein. For example, one could have different 96 well plates, one of which has liver cells, one of which has lung cells, and one of which has heart cells heart cells, for example, and ship these as a kit with reagents and media. The end user, would then add things to be tested, for example, into the wells. Another example includes screening using a high density array of chemicals on a film which is then washed with various solutions containing compositions, such as cells or other things, which then give an indicator if they interact with something on the chip.

Also disclosed are chips where at least one address is a variant of the sequences or part of the sequences set forth in any of the nucleic acid sequences, peptides, or cells disclosed herein. Also disclosed are chips where at least one address is a variant of the sequences or portion of sequences set forth in any of the peptide sequences disclosed herein.

g) Computer Readable Media

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable media comprising the sequences and information regarding the sequences set forth herein.

h) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include nucleic acids encoding the desired molecules or modified ES cells discussed in certain forms of the methods, as well as the buffers and enzymes required to use them. Other examples of kits, include cells derived by the methods described herein useful for toxicity screening. These cells can represent a variety of terminally differentiated cells that give a relevant profile of the drug being screened. The cells could, for example, still comprise the marker or could have the marker excised. Since the methods allow the use of a pluripotent cell as the starting cell, multiple cell types all derived from a common pluripotent cell and thus sharing a common genotype can be generated. Kits, can include, for example, plates, such as 96 well plates, which can be coated with the compositions disclosed herein.

### B. METHODS OF USING DIFFERENTIATED CELLS

The methods for making the modified stem cells as disclosed herein can produce cells which are suitable for in vivo methods and/or ex vivo methods and/or in vitro methods. For example, the activated/dominant negative transforming gene strategy, for example, can be best suited to in vitro applications but would not be as desirable for cell therapy because the marker, such as the transforming gene, would remain within the cell. On the other hand CRE/lox is suitable for cell therapy because the marker, such as a transforming gene, is excised from the final cell. Furthermore, for in vivo mechanisms the marker can be placed on an extrachromosomal cassette, such as a mammalian artificial chromosome, which can then be removed entirely from the final cells using a variety of mechanisms.

1. Reconstituted Immune System

Disclosed herein are methods and compositions capable of generating and modifying any desired human cell type. For example, disclosed is the in vitro reconstitution of the human immune system. Monoclonal antibodies currently are produced in mice by a three-step process. The mouse is first inoculated with the desired antigen. After a few days, its spleen is removed and the immune cells residing in the spleen are fused with a mouse B cell lymphoma line. This serves to immortalize the B cells in the spleen. These are then cultured and the fusion that is producing the appropriate antibody is selected.

Mouse monoclonal antibodies are poor therapeutics in humans since they are recognized as foreign and destroyed. Monoclonal antibodies that are currently being used for therapies, such as Herceptin® for breast cancer, are humanized or chimerized to minimize these problems, but they are not completely eliminated. Fully human monoclonal antibodies are the solution. Unfortunately, this would mean inoculating people with the antigen. This has been both unpopular and unsuccessful, in the few instances where it has been attempted. As disclosed herein, directed differentiation of stem cells will allow the selection of a matched set of human immune cells: B, T and macrophage lines. This can only be accomplished from stem cells since the B, T, and macrophage cells should be from the same genetic background in order to function correctly. When the appropriate cells are established, they can be cultured together to produce an in vitro immune system. Antigen incubated in the system can be processed and presented to the B cells correctly, expanding the cognate cells. With time in culture, these cells can proliferate preferentially or selectively, comprising a larger percentage of the total B cell population. These cells can then be cloned and the appropriate antibody producing cell can be selected. Because they are transformed, they can be characterized, frozen, and then expanded indefinitely, producing fully human monoclonal antibodies. This system can dramatically expand the applicability of monoclonal antibodies for therapy.

2. Toxicology Testing

The desire of the pharmaceutical industry to drive down the staggering cost of new drug discovery and development has forced an examination of the factors that cause drug candidates to fail. After efficacy problems, the most common reason for failure is toxicity (van de Waterbeemd, H, Gifford, E. (2003) Nat. Rev. Drug Disc. 2, 192-204). Even more problematic are compounds that go onto the market, only to be withdrawn due to unrecognized toxicities. Troglitazone and trovafloxacin are well known examples of compounds which were pulled or whose use was severely curtailed due to liver toxicity, grepafloxacin had problems with muscle toxicity, terfenadine and astemizole were pulled due to cardiac toxicity (Suchard, J. (2001) Int. J. Med. Toxicol. 4, 15-20).

Ideally, the toxic properties of new compounds can be recognized and avoided early in development. ACTIVTox, based on a human liver cell line, is designed to provide a high throughput, metabolically active platform for the development of structure toxicity relationships. Compounds are screened through a battery of tests at multiple concentrations to develop a structural ranking that can be used by the chemists to direct the next round of synthesis. In this way, the toxic properties of a compound can be minimized while the therapeutic properties are maximized.

By developing a panel of related cell lines, the idea of ACTIVTox can be generalized. New compounds can be tested against a panel of matched, non-transformed cell lines in a high throughput system, raising the probability of success in clinical trials. Using the methods described herein, the panel can consist of cell lines, representing a number of tissues, matched as closely as possible. These cells would constitute a set of tissue samples from a single individual, minimizing problems with differences in genetic background.

Predictive toxicology using the disclosed method can also be performed with a larger cell collection. Disclosed are methods of toxicology testing on heart, neuron, intestine, kidney, liver, muscle, or lung lines. These lines can be produced and screened in the same toxicity assays using the same compounds, as those which are used for liver.

An example is beating heart cell cultures. A major concern among pharmaceutical companies is the phenomenon known as QT prolongation, which can lead to heart arrythmias and possibly death (Belardinelli, L., et al. Trends in Pharmocol. Sci. 24, 619-625, 2003). Several compounds, such as terfenadine, were withdrawn from the market for this serious side effect. Currently, it is difficult to test for QT prolongation except in animals or people, since it is an electrical phenomenon. Beating heart cell cultures would allow a direct test for this problem.

By testing the same compounds in the same assays using many different cell types, a clear picture of the toxic potential of new compounds can be determined before testing in humans. This will have a dramatic effect on the cost and speed of new drug development since clinical testing is by far the most expensive phase.

3. Specific Target Cells for Discovery Applications

a) Dopamine Specific Neurons

The use of the disclosed methods and compositions also allows the development of specific cell types for drug discovery applications. Currently, new drugs are frequently tested on cells that have been genetically manipulated to contain the target of interest because the natural target-containing cell is unavailable. An example is dopaminergic neurons. Many neuroactive drugs are directed against the dopamine receptor, such as the tricyclic antidepressants or dopamine reuptake inhibitors for drug addiction. The availability of an unlimited and reproducible supply of the specific cell type of interest, such as dopaminergic neurons uncontaminated by any other cell type, are disclosed herein.

4. Knockouts for Target Validation

The use of the disclosed methods and compositions, in combination with gene targeted, homologous recombination, allows the development of cells with a particular gene deleted or modified. A central problem in drug development is the validation of therapeutic targets. This is the determination of whether a particular protein, when blocked or activated by a drug, will in fact deliver the desired therapeutic effect. Knockout or knock in mice are frequently used in this application (Zambrowicz, B P, et al. Nat. Rev. Drug Disc. 2, 38-51, 2003). The disclosed cells and cell lines, which have been produced as disclosed herein, will provide similar validation opportunities in vitro. A specific example is the knockout of the human low density lipoprotein receptor. The LDL receptor is used as an entryway for a number of human viruses, including the human hepatitis B virus. Using the techniques of homologous recombination in the cells disclosed herein, such as stem cells, the LDL receptor gene can be damaged, such that no LDL receptor protein is synthesized. Using directed differentiation in these cells, human hepatocytes without the LDL receptor can be created. These cells can be used to examine the role of the LDL receptor in HBV infection. If, for example, these cells were uninfectable with HBV, the LDL receptor would be declared to be a validated target for anti HBV therapies. Similar strategies could be devised to create gain of function or loss of function mutations for other purposes. Using the same example as above, the LDL receptor could be activated in cells that normally do not express this protein.

5. Ex Vivo Cell Therapy

a) Liver Assist Device

Disclosed is a liver assist device based on the liver cell lines disclosed herein. There are about 5,000 liver transplantations carried out in the United States each year. There are currently about 17,000 on the waiting list. About 1500 die on the list each year.

Currently, there is no means to support a patient who has entered into end stage liver disease, such as hemodialysis for kidney patients. Because of the liver's ability to regenerate, support for this short, crucial period can allow the patient to survive, either until a suitable organ is available or, in the best of circumstances, with their own liver.

A liver assist device in animals and on 52 patients in the United States and Great Britain has been developed and tested (Sussman, N L, et al., (1992) Hepatology 16, 60-65; Sussman, N L, et al., (1994) Artificial Organs 18, 390-396; Millis, J M, et al., (2002) Transplantation 74, 1735-1746). In this device, a hollow fiber cartridge, as is used in kidney dialysis, is filled with a human liver cell line that carries out the function of the liver. The cells are separated from the patient's immune system by the cellulose acetate fibers. Blood is pumped through the lumen of the fibers, small molecules diffuse through the fibers to the cells, where they are appropriately metabolized. The device is safe and while trials of sufficient power to prove its effectiveness have not been carried out, anecdotal evidence suggests that it is able to save lives. Other similar devices, using animal hepatocytes, also appear to be effective (Hui, T, et al., (2001) J. Hepatobiliary Pancreat Surg. 8, 1-15).

A practical problem arises in the source of the hepatocytes to fill the device. In order to be effective, each device requires about 200 g of cells, 15 to 20% of the total liver mass. Hepatocytes, despite their regenerative capabilities in vivo, do not divide to any extent in culture, even after decades of research on this topic. The statistics described in the opening paragraph are not encouraging in using human livers to supply cells for support devices. Transplantation is totally organ limited. The use of animal livers can supply sufficient cells but requires the constant harvest of new organs and presents problems of reproducibility and quality control. This problem has been approached by employing a human liver cell line, which is immortalized and could be frozen in cell banks (Sussman, N L & Kelly, J H. (1995) Scientific American: Science and Medicine 2, 68-77). These cells can supply a constantly renewable, reproducible and unlimited supply of devices.

Unfortunately, the tumor-derived source of these cells has presented acceptance and regulatory problems for its use in human therapy. The disclosed hepatocytes produced from the compositions and methods disclosed herein can circumvent this hurdle.

6. Genetically Matched Cell Lines

Genetically matched cell lines can be used for gene expression studies and proteomic studies since the genetic noise level can be dramatically reduced.

A major drawback to use of cells in culture, prior to the disclosed cells, to study gene expression is that the cells do not have the same genetic background. Different sets of genes are expressed at different levels in different individuals. This has both a genetic and environmental component. Moreover, most cells in culture are derived from tumors, which are, by definition, genetically abnormal and usually contain multiple inversions, duplications and completely duplicated or missing chromosomes.

A set of cells that were isolated from the same stem cell would be that same as having tissue samples from an individual. The genetic background of cells from the liver and the intestine, for example, would be the same. This allows for a much clearer determination of tissue specific expression of genes and proteins, since individual variability is eliminated. The disclosed methods and compositions can be used to produce genetically matched cells of a specific cell type from any cell disclosed herein, such as stem cells, from any source, such as any unique individual.

7. Identification of Developmental Pathways and Control

As described earlier, transcription factors act combinatorially to effect tissue specific gene expression. The disclosed compositions and methods can be used to identify cell stages that activate certain genes specific for a given cell type. Using the hepatocyte as an example, albumin is primarily a product of the adult hepatocyte. Several transcription factors are known to regulate its expression. One such factor is C/EBP, a factor in the regulation of many genes involved in intermediary metabolism (Darlington, G J, (1998) J. Biol. Chem. 273, 30057-30060). Using the promoter for C/EBP in the EG system, for example, one can identify cells that activate this gene. One of these is the hepatoblast, a precursor to the hepatocyte. By then selecting a gene whose expression regulates C/EBP, we can follow the developmental pathway backwards to the origin, stepwise.

### C. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular modified ES cell is disclosed and discussed and a number of modifications that can be made to a number of molecules including the modified ES cell are discussed, specifically contemplated is each and every combination and permutation of modified ES cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that there are many different compositions and method steps disclosed herein and each and every combination and permutation for each composition and method as disclosed herein is contemplated and disclosed. For example, there are lists of transformation genes, promoters, cell types, recombinase combinations, modified stem cells, markers, cell specific genes, and each combination of each of these singularly or in total, is disclosed, which provides many thousands of specific embodiments and sets of embodiments. Once the lists and pieces are disclosed, the combinations are also disclosed without specifically reciting each combination.

Furthermore, it is understood that unless specifically indicated to the contrary or unless understood as being contrary to the skilled artisan, where one specific embodiment is discussed, such as a Ras transformation gene, then all other transformation genes are also disclosed for that recitation or embodiment, and likewise for each composition and method step disclosed herein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, primary culture, or cultures derived from such cells unless specifically indicated. A “culture” refers to a composition comprising isolated cells of the same or a different type.

A cell line is a culture of a particular type of cell that can be reproduced indefinitely, thus making the cell line “immortal.”

A cell culture can be a population of cells grown on a medium such as agar.

A primary cell culture is a culture from a cell or taken directly from a living organism, which is not immortalized.

The term “pro-drug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

The term “metabolite” refers to active derivatives produced upon introduction of a compound into a biological milieu, such as a patient.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Nucleic acid segments for use in the disclosed method can also be referred to as nucleic acid sequences and nucleic acid molecules. Unless the context indicates otherwise, reference to a nucleic acid segment, nucleic acid sequence, and nucleic acid molecule is intended to refer to an oligo- or polynucleotide chain having specified sequence and/or function which can be separate from or incorporated into or a part of any other nucleic acid.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide can be independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. For example, disclosed are the cells produced by the disclosed methods. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequences disclosed herein and a sequence controlling the expression of the nucleic acid.

Also disclosed are nucleic acid molecules produced by the process comprising linking in an operative way a nucleic acid molecule comprising a sequence having 80% identity to the sequences disclosed herein, and a sequence controlling the expression of the nucleic acid.

Disclosed are nucleic acid molecules produced by the process comprising linking in an operative way a nucleic acid molecule comprising a sequence that hybridizes under stringent hybridization conditions to the disclosed sequences and a sequence controlling the expression of the nucleic acid.

Disclosed are nucleic acid molecules produced by the process comprising linking in an operative way a nucleic acid molecule comprising a sequence encoding a peptide disclosed herein and a sequence controlling an expression of the nucleic acid molecule.

Disclosed are nucleic acid molecules produced by the process comprising linking in an operative way a nucleic acid molecule comprising a sequence encoding a peptide having 80% identity to a peptide disclosed herein and a sequence controlling an expression of the nucleic acid molecule.

Disclosed are nucleic acids produced by the process comprising linking in an operative way a nucleic acid molecule comprising a sequence encoding a peptide having 80% identity to a peptide disclosed herein, wherein any change from the peptide sequence are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids. Combinations of different cells produced by the methods described herein are also disclosed. Also combinations of cells produced by the methods described herein mixed with other cells are also provided. These cells can have various purities based on the particular need or application.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting, a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

Disclosed are any of the stem cells disclosed herein produced by transforming the cells with the nucleic acids disclosed herein. Also disclosed are any of the cells produced by the methods disclosed herein, such as the methods for isolating selecting a specific cell type and using the disclosed modified stem cells.

### E. METHODS OF USING THE COMPOSITIONS

1. Methods of Using the Compositions as Research Tools

The disclosed compositions can be used in a variety of ways as research tools.

The compositions can be used for example as targets in combinatorial chemistry protocols or other screening protocols to isolate molecules that possess desired functional properties related to the specific cell type.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, a particular gene in a particular cell type disclosed herein. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein. Similarly, a stem cell, such as a pluripotent stem cell, can be used to knock out a gene to create a transgenic animal and the same cell can be used in methods described herein to create cell lines that can be compared to the animal in various assays.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### F. SPECIFIC EMBODIMENTS

Provided herein is an isolated human pluripotent stem cell derived from gonadal ridge or testes of fetal or embryonic material that can be maintained without a feeder layer for at least 20 passages, wherein the cell is grown in a culture medium that has not been conditioned by a feeder layer, maintains the potential to differentiate into derivatives of endodermal, mesodermal, and ectodermal cells throughout the culture, and maintains a normal karyotype.

The stem cell can be derived from a primordial germ cell (PGC). The stem cell can be a PC™. The stem cell can stain positive for the SSEA-1 antigen, stain negative for SSEA-4 antigen, and stain positive for alkaline phosphatase. The stem cell can be directly contacting a solid substrate. The culture medium can comprise an amount of oncostatin M sufficient to maintain the stem cell without a feeder layer for at least 20 passages. The culture medium can comprise an amount of forskolin sufficient to maintain the stem cell without a feeder layer for at least 20 passages. The culture medium can comprise an amount of FGF sufficient to maintain the stem cell without a feeder layer for at least 20 passages. The culture medium can comprise an amount of stem cell factor (SCF) sufficient to maintain the stem cell without a feeder layer for at least 20 passages.

Also provided is a composition comprising the herein provided isolated stem cell growing on a solid substrate such as plastic, glass or the like without a feeder layer.

Also provided herein is a culture medium for growing stem cells in the absence of a feeder layer, comprising a base medium suitable for growing stem cells and an amount of oncostatin M sufficient to maintain the stem cell without a feeder layer for at least 20 passages. The culture can comprise at least 5 uM forskolin. The culture can comprise at least 5 ng per ml FGF. The culture can at least 5 ng per ml stem cell factor (SCF). The culture medium can comprise at least 5 uM of oncostatin M.

Also provided is a composition comprising an isolated stem cell in the herein provided culture medium. In one aspect, the stem cell does not contact a feeder layer.

Also provided herein is a method of isolating a pluripotent stem cell, comprising providing primordial germ cells (PGCs) from a human embryo; culturing said cells directly on a solid substrate in the herein provided culture medium; selecting cells that exhibit the following characteristics: maintains a normal karyotype for at least 20 passages and maintains the potential to differentiate into derivatives of endodermal, mesodermal, and ectodermal cells throughout the culture. Also provided is an isolated pluripotent human stem cell derived by the herein provided method. The pluripotent cell can be a clone. In one aspect, the cell does not comprise Neu5Gc.

Also provided is method of deriving terminally differentiated cells, comprising directing the differentiation of the herein disclosed stem cells using conditional immortalization.

Also provided is an isolated pluripotent stem cell derived from gonadal ridge or testes of fetal or embryonic material that can be maintained without a feeder layer for at least 20 passages, wherein the cell: is grown in a culture medium that has not been conditioned by a feeder layer, maintains the potential to differentiate into derivatives of endodermal, mesodermal, and ectodermal cells throughout the culture, and maintains a normal karyotype.

Also provided is an isolated pluripotent stem cell that can be maintained without a feeder layer for at least 20 passages, wherein the cell: maintains the potential to differentiate into derivatives of endodermal, mesodermal, and ectodermal cells throughout the culture, stains negative for SSEA-4 antigen, and maintains a normal karyotype. The isolated stem cell can stain positive for the SSEA-1 antigen. The isolated stem cell can be grown in a culture medium that has not been conditioned by a feeder layer.

Also provided is an isolated stem cell that stains negative for the SSEA-4 antigen. The isolated stem cell can stain positive for the SSEA-1 antigen. The isolated stem cell can maintain a normal karyotype. The isolated stem cell can maintain the potential to differentiate into derivatives of endodermal, mesodermal, and ectodermal cells throughout the culture. The isolated stem cell can stain positive for alkaline phosphatase. The isolated stem cell can be derived from a primordial germ cell (PGC). The isolated stem cell can stain negative for Neu5Gc. Also provided is a composition comprising the isolated stem cell and at least 5 uM of oncostatin M.

Also provided is a method of isolating an ocostatin-independent stem cell (OISC), comprising providing a PC™; culturing said cells in medium comprising at least 5 ng per ml FGF and comprising less than 0.001, 0.01, 0.05, 0.1, 1 ng per ml oncostatin M and SCF; selecting cells that stains positive for alkaline phosphatase, SSEA-1, Oct-4, and Nestin; and isolating said OISC.

Also provided is a cell produced by any of the herein provided methods.

Also provided is an isolated stem cell that can be maintained without a feeder layer for at least 20 passages, wherein the cell maintains the potential to differentiate into derivatives of endodermal, mesodermal, and ectodermal cells throughout the culture, stains negative for SSEA-4, stains positive for alkaline phosphatase, SSEA-1, Oct-4, and Nestin, and maintains a normal karyotype. The isolated stem cell can be grown in a culture medium that has not been conditioned by a cell line or feeder layer. The stem cell can be an ocostatin-independent stem cell (OISC)

Also provided is an isolated stem cell wherein the cell stains positive for alkaline phosphatase, SSEA-1, Oct-4, and Nestin and stains negative for the SSEA-4 antigen. The stem cell can be an ocostatin-independent stem cell (OISC), The isolated OISC can maintain a normal karyotype. The isolated OISC can maintain the potential to differentiate into derivatives of endodermal, mesodermal, and ectodermal cells throughout the culture. The isolated OISC can be derived from fetal gonadal tissue, e.g. a primordial germ cell (PGC).

Also provided is a method of producing a homogenous population of neural progenitor cells (NPCs), comprising: providing an oncostatin-independent stem cell (OISC), culturing said cells in medium comprising FGF and retinoic acid; selecting cells that exhibit the following characteristics: stain positive for Nestin, stains negative for alkaline phosphatase and Oct-4; and isolating said NPCs. Also provided is an homogenous population of neural progenitor cells (NPCs) produced by the provided method.

Also provided is a method of producing a homogenous population of muscle progenitor cells (myoblasts), comprising, provided an oncostatin-independent stem cell (OISC), culturing said cells in medium comprising FGF, forskolin and bromo-cyclic AMP; selecting cells that exhibit the following characteristics: stain positive for alpha-actinin, stains negative for alkaline phosphatase and Oct-4; and isolating said myoblasts. Also provided is an homogenous population of muscle progenitor cells (myoblasts) produced by the provided method.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Establishment of the Human Embryonic Germ Cell Culture Hay1

Using the techniques defined by Matsui, et al. ((1992) Cell 70, 841-847), a human EG line was established. Briefly, the gonadal ridges were dissected from a 10 week male fetus, dissociated with trypsin-EDTA and plated onto irradiated STO feeder layers. Cells were fed daily with DMEM, 15% fetal bovine serum, supplemented with non-essential amino acids and β-mercaptoethanol, 60 ng/ml human Stem Cell Factor (SCF), 10 ng/ml human Leukemia Inhibitory Factor (LIF) and 10 ng/ml human basic Fibroblast Growth Factor (FGF). On day 5, one of the two flasks was stained for alkaline phosphatase. Many positive cells were observed. Cells were passaged with trypsin-EDTA on day 6 and split 1 to 4 onto fresh irradiated STO layers. This process was repeated, following alkaline phosphatase at each passage. At passage 5, several vials of cells were frozen in DMEM, 15% fetal bovine serum, 10% dimethylsulfoxide, using a controlled rate freezer. Cells are routinely passaged now on mitomycin C treated STO layers.

a) Characteristics of Hay1

Hay1 cells, both on feeder layers and on plastic, as described below, grow as elongated cells resembling migratory primordial germ cells (Shamblott et al. (1998) Proc. Natl. Acad. Sci. 95, 13726-13731; Turnpenny et al. (2003) Stem Cells 21, 598-609). Hay1 displays morphology identical to the cells described by Turnpenny, et al. In addition to alkaline phosphatase, the cells stain positively for SSEA-1, TRA 1-60 and TRA 1-80. Determination of karyotype and multi-tissue tumor formation is underway. When switched to low adherence plastic in the absence of feeders or hormone supplements, they readily form cystic embryoid bodies. When these embryoid bodies are re-plated in tissue culture plastic, the cells exhibit dramatically different morphology and lose expression of alkaline phosphatase.

b) Culture of Hay1 in defined conditions

The use of feeder layers complicates the use of stem cells for a variety of applications. Use of feeder layers dramatically raise the background in standard in vitro toxicology assays, such as MTT or resazurin reductions confounding the results. Hay1 can be grown routinely under defined conditions. Standard medium consists of KO-DMEM, 15% KO-serum replacement, glutamine, nonessential amino acids, β-MeSH, 10 ng/ml oncostatin M, 10 ng/ml SCF and 25 ng/ml FGF-2. Using this medium, Hay1 continues to express the markers listed above and doubles approximately every three to four days. This is slightly slower than their doubling on feeder layers.

c) Hay1 expresses Oct 4 and Nanog

While surface markers and alkaline phosphatase are convenient markers for stem cells, it has become clear that expression of the transcription factors Oct 4, Sox2, and Nanog are fundamental characteristics of pluripotent stem cells (Rodda et al. (2005) J. Biol. Chem. 280, 24731-24737; Chambers et al. (2003) Cell 113, 643-655). Hay1 was examined for expression of these factors using real time RT-QPCR. Expression of cells under standard defined conditions was compared to that in cells that have been subjected to differentiation via EB formation followed by culture in Med3 (Kelly and Sussman, (2000) J. Biomol. Screen. 5, 249-254), a medium that is a mixture of Weymouth's MAB, Ham's F12 and William's E. It also contains 5% defined calf serum (Hyclone). Actin was used as a standard. The results show that both Oct 4 and Nanog are expressed in Hay1 and that expression falls dramatically upon differentiation.

d) Hay1 is dependent on Oncostatin for growth

Growth of Hay1 was examined under various conditions known to affect stem cell growth and differentiation. When each of the three peptide hormone factors (One M, SCF, FGF-2) was removed individually from the medium, each had some effect on growth. However, removal of oncostatin M completely arrested the growth of the cultures and they became alkaline phosphatase negative within several days.

e) FGF induces Oct 4 and Nanog

Removal of FGF-2 from the culture had a slight negative effect on growth of the culture and an effect on morphology, with the cells becoming flatter and more spread out on the dish. Cultures were examined for Oct 4 and Nanog expression after FGF-2 withdrawal and a dramatic reduction in expression was observed. Replacement of FGF-2 returned Oct 4 expression to its former level. Since Oct 4 controls Nanog expression (Rodda et al. (2005)), it was expected that induction of Oct 4 would also raise nanog, and this is what was observed.

f) Zeocin sensitivity

In preparation for the establishment of the frt insert line, the sensitivity of Hay1 to zeocin was tested. A standard titration curve indicated that a concentration of 75 μg/ml will be an effective selection concentration.

### Example 2

### Establishment of the Human Embryonic Germ Cell Line PCHay1D

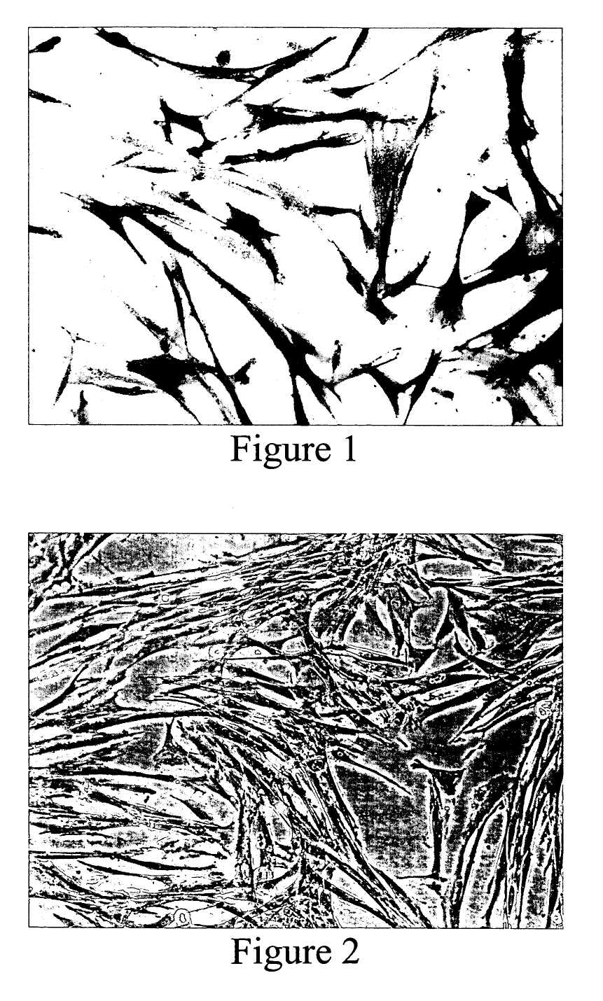

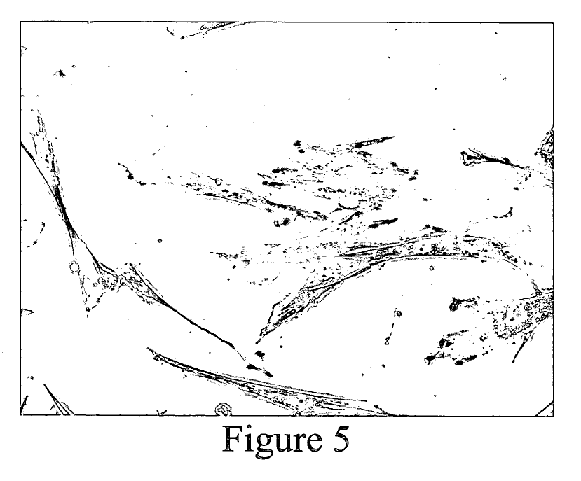

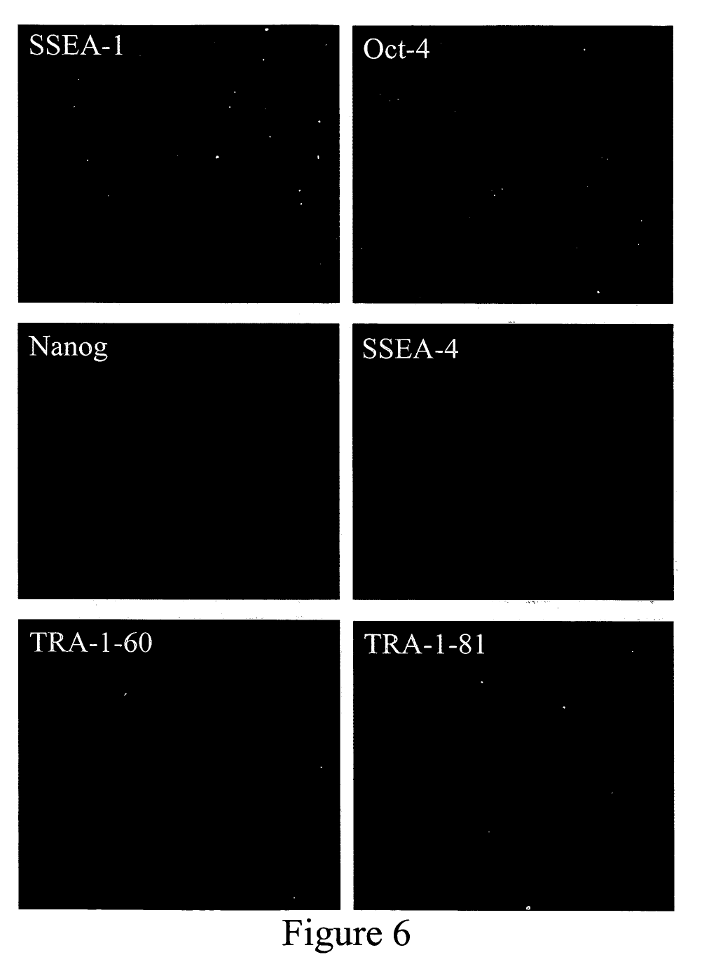

As shown in FIGS. 1 and 5, PCHay1D is a clone established from the Hay1 cell culture. Hay1 cells were trypsinized and diluted, then plated in five 96 well plates such that each well of the plate should receive one cell. After three weeks in cell culture medium containing 10 ng/ml human oncostatin M, 10 ng/ml human stem cell factor, 25 ng/ml basic FGF-2, 10 μM forskolin, 5% defined calf serum, colonies arose in approximately half the wells of the plates. Approximately 10% of these were strongly alkaline phosphatase positive (AP+). Twenty four individual colonies were selected and replated into duplicate 24 well plates. After one week, one of these plates was stained for alkaline phosphatase. Hay1D had both strong growth and alkaline phosphatase activity.

PCHay1D is positive for SSEA1, Tra-1-60, Tra-1-81, Oct 4, Nanog and Cripto. It is SSEA-4 negative.

### Example 3

### Establishment of PCs

To date, ten independent cell lines have been derived from human fetal gonadal tissue in the absence of a feeder layer using oncostatin M. The PCs were derived as follows. Tissue of varied ages within the first trimester was collected from Planned Parenthood. It is preferable to identify and isolate gonadal ridge or testes from the embryo. Out of about 200 grams of tissue, the embryo is about 1 gram, and the gonadal ridge or testes represent milligrams of tissue. The tissue was homogenized. A piece of tissue was placed in trypsin/EDTA and incubated 5 min. The trypsin was then inactivated by adding culture medium containing serum, which is described elsewhere herein. Soybean trypsin inhibitor can also be used. The tissue was triturated to break up and release the cells, which were examined under the microscope. The cell suspension was then plated in a T25 culture dish with 5 mls of culture medium comprising Knockout DMEM (Invitrogen), 15% knockout serum replacement (Invitrogen), 10 ng per ml short (32,000 Mr) human oncostatin M, 10 ng/ml human stem cell factor (SCF), 25 ng/ml human FGF-2, 10 uM forskolin, 1 mM glutamine, 0.1 M mercaptoethanol, and 0.1 mM non-essential amino acids.

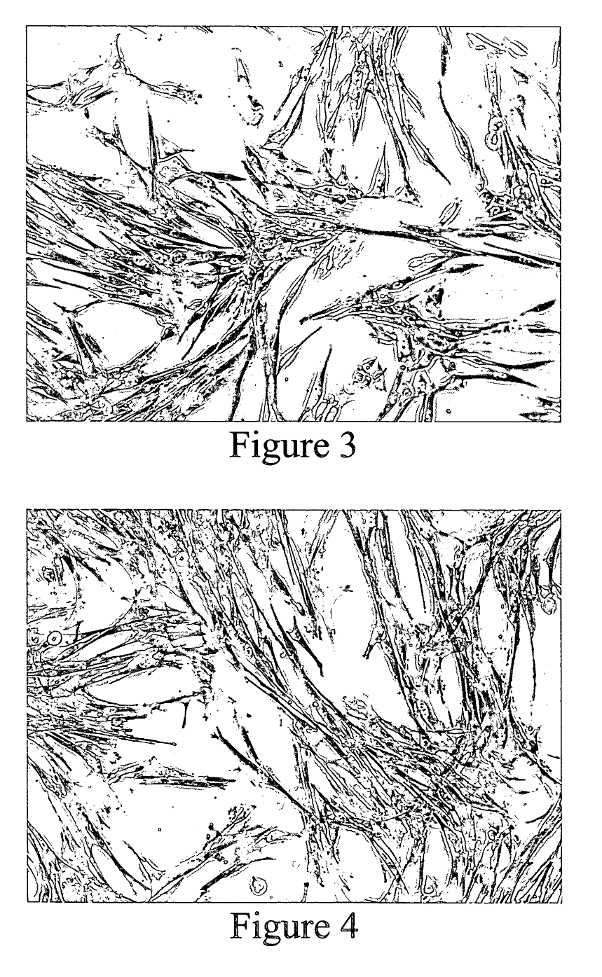

PC1 (FIG. 1), PC3, PC9 (FIG. 3) and PC10 (FIG. 4) were all established from male fetal gonads (testes). PC14 was established from female fetal gonads. The tissue was identified and dissected then incubated in 50011 of trypsin EDTA for 5 minutes at 37° C. The tissue was further disrupted by repeated pippeting, then plated in duplicate 25 cm² cell culture flasks in medium containing either 10 ng/ml human oncostatin M or 10 ng/ml human LIF, 10 ng/ml human stem cell factor, 25 ng/ml basic FGF-2, 10 μM forskolin, 15% Knockout Serum Replacement (Invitrogen).

Primordial germ cells, recognized by size and alkaline phosphatase staining, attached and proliferated over the first few days of culture. They originally have the classic elongated shape equated with migratory germ cells. In the presence of LIF, these cells assumed a more rounded morphology and lost alkaline phosphatase after 7 to 10 days in culture. In the presence of oncostatin M, the same cells proliferated, maintained alkaline phosphatase activity and assumed a larger, stick like morphology. The cells expressed high levels of oncostatin M receptor on their surface. Growth was inhibited by WHI—P 131 and AG490, both STAT inhibitors. Removal of oncostatin resulted in rapid loss of alkaline phosphatase activity.

The cells were trypsinized and replated into larger flasks to expand the population. Cells were frozen in liquid nitrogen after three passages.

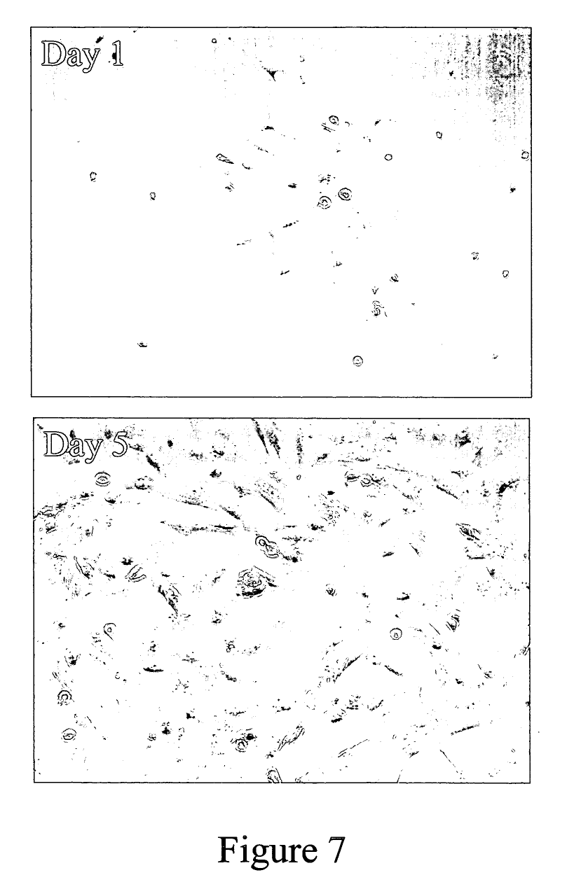

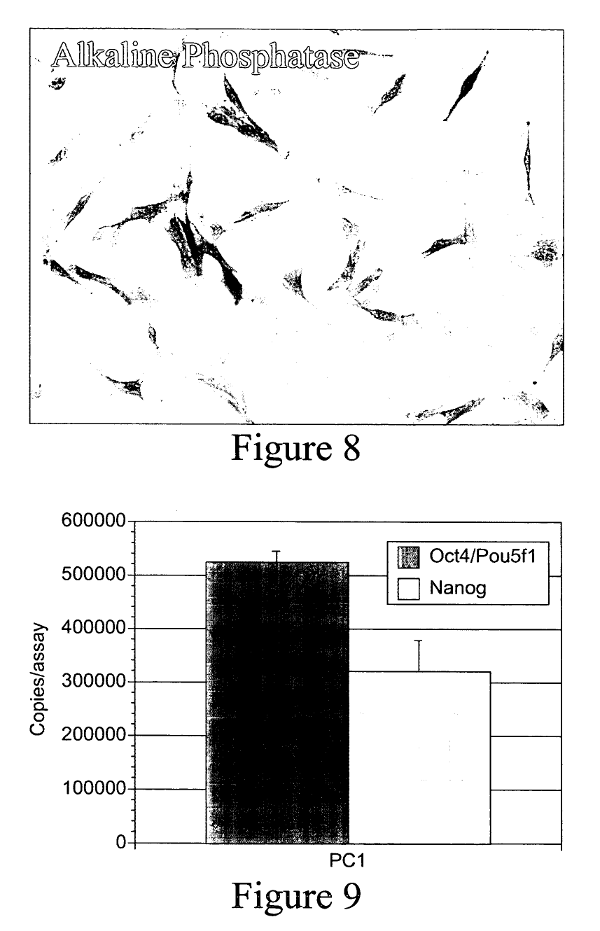

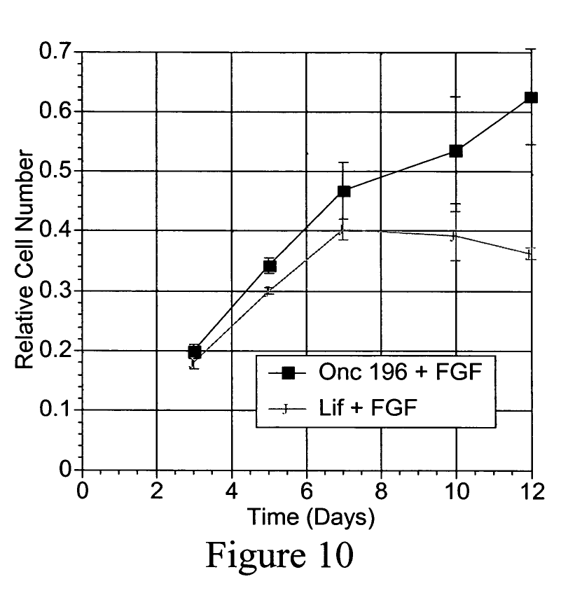

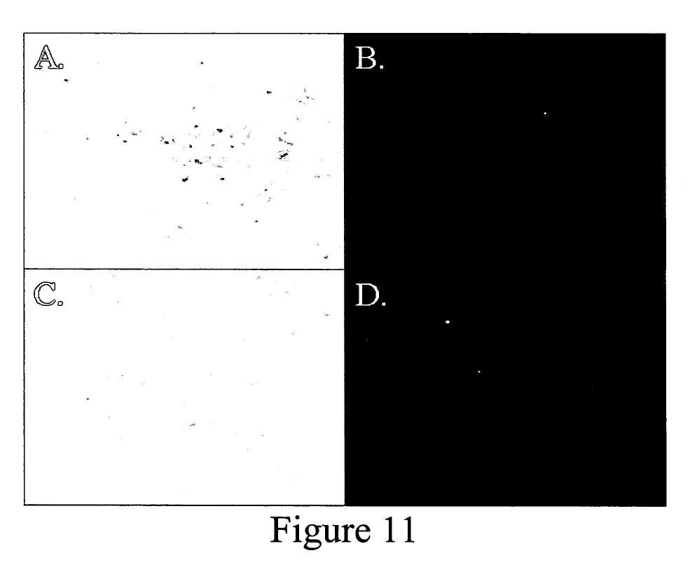

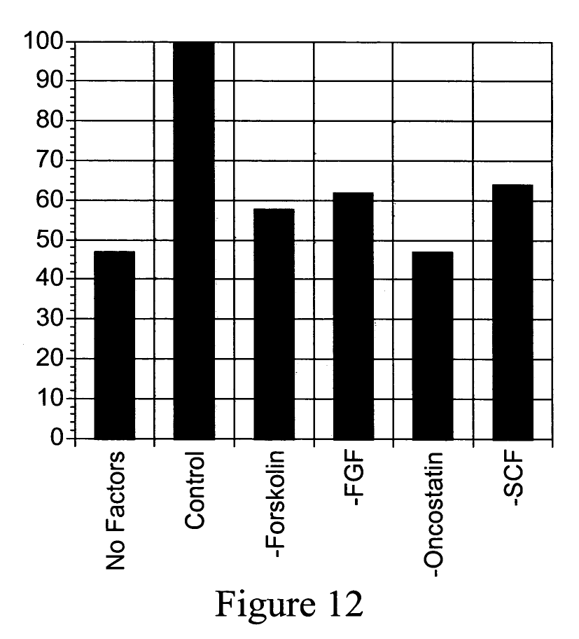

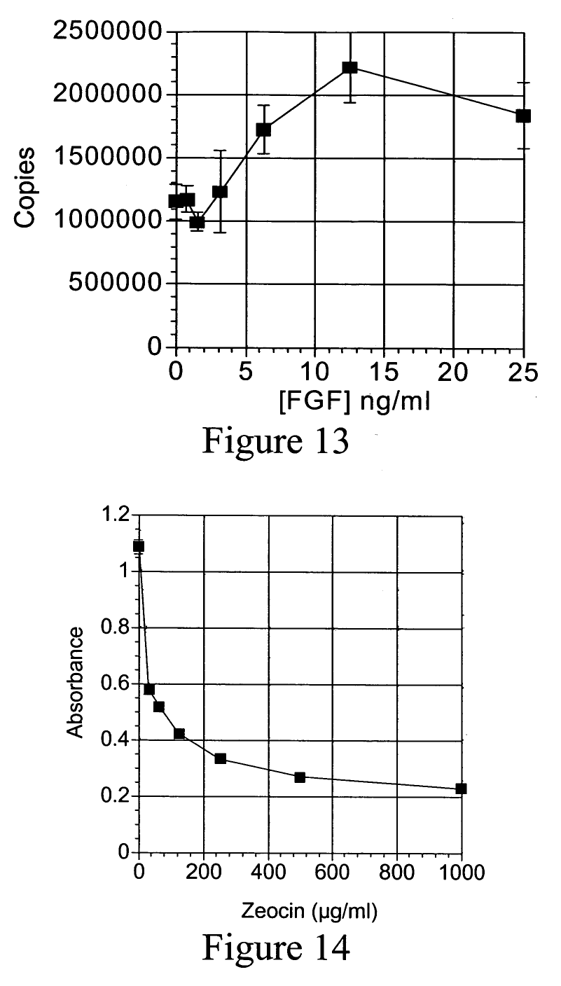

PCs are all positive for SSEA1, Tra-1-60, Tra-1-81, Oct 4, Nanog, Sox2, Tcl1, Tbx3, Cripto, Stellar, and Daz1, and are uniformly negative for SSEA-4 (see FIG. 1). PCs also stain positively for alkaline phosphatase (FIG. 1) and maintain a normal karyotype. As shown in FIG. 7, there is massive proliferation of PCs between the day 1 and day 5 after explant.

### Example 4

### Establishment of Non-Xenogenic PCs

Human embryonic cell suspension can be provided as described above and then plated in duplicate 25 cm² cell culture flasks in the defined medium of Table 1, 10 ng/ml human short (32,000 Mr) oncostatin M, 10 ng/ml human stem cell factor, 25 ng/ml FGF-2, 10 μM forskolin. One of the flasks can be stained for alkaline phosphatase on day 2 after plating. In each case, there are AP+cells. These can be trypsinized and replated into larger flasks to expand the population. Cells can be frozen in liquid nitrogen after three passages.

Non-Xenogenic PCs are positive for SSEA1, Tra-1-60, Tra-1-81, Oct 4, Nanog and Cripto and are uniformly negative for SSEA-4. Non-Xenogenic PCs cells also stain positively for alkaline phosphatase. Non-Xenogenic PCs are also negative for Neu5Gc and will not be immunotargeted by antibodies specific for Neu5Gc. Non-Xenogenic PCs can therefore be suitable for human in vivo use.

The PCs and Non-Xenogenic PCs can be used in the methods described herein to produce the more differentiated cells described herein.

For example, hepatocytes were produced by treating PCs with hepaocyte growth factor and FGF4 for two weeks, then switching them into the hepatocyte cell culture medium, which contained insulin, selenium, transferring, and dexamethasone. This method can produce cell populations comprising at least 80% heptocytes in the absence of cell sorting.

### Example 5

### Nestin Positive Stem Cells and Directed Differentiation Thereof

PCs were cultured in medium containing oncostatin M, stem cell factor and FGF-2 to maintain their undifferentiated state. When these factors are removed and the cells are cultured in medium containing insulin, selenium, transferrin (ITS), FGF-2 and retinoic acid, they lose markers of pluripotentcy, such as Oct4 and alkaline phosphatase and become Nestin positive. These cells, referred to herein as neural progenitor cells (NPCs), can be expanded quite dramatically. When sonic hedgehog (Shh) and Noggin are added to the medium, cells displaying markers of motor neurons (MN) become apparent within a few days and become the dominant cell type in the population over two weeks, after the hormones are withdrawn.

Moreover, because the differentiation is carried out in situ rather than using the formation of embryoid bodies, a large percentage of the cells make the final transition to motor neurons. Immunofluorescence and QPCR can be used to characterize the cells. Early neural transition markers are nestin, Sox1 and Pax6. Intermediate markers are Pax7, MAP2, Lim3, Nk×6.1 and Olig2. Definitive markers are HB9, ChAT and Tuj1.

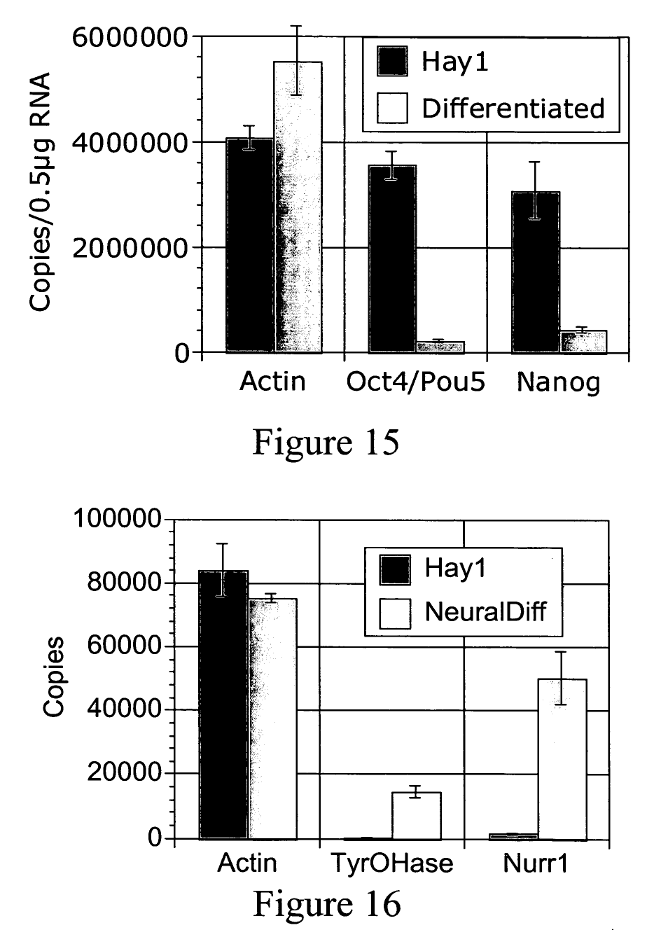

When oncostatin M and SCF are removed from PC cultures and they are maintained in the presence of FGF-2 to drive division, the cells quickly lose alkaline phosphatase activity. However, as shown in FIG. 15, Oct4 and Nanog expression decline but are still expressed.

When the cells are cultured in FGF-2 plus retinoic acid, after two weeks in culture they develop certain aspects of neuronal differentiation. Both Nurr1 and tyrosine hydroxylase could be detected via RT-PCR in these populations (FIG. 16).

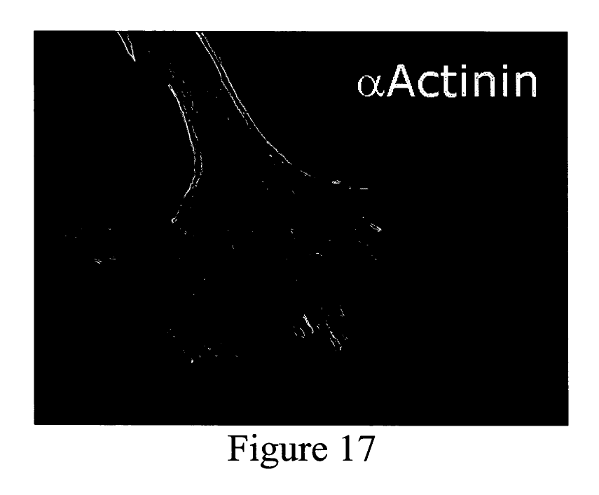

When the cells are cultured in the absence of oncostain and SCF but including FGF-2, forskolin and bromo-cyclic AMP, the cells develop aspects of smooth muscle differentiation. Shown in FIG. 17 is a cell after two weeks in such a medium, stained with alpha-actinin and with apparent sarcomeres.

### Example 6

### PC Cell Line EG1 Transfected with a CMV Promoter Driven GFP (pmaxGFP)

Cells were removed from the culture dish with trypsin/EDTA. The cell layer was washed with 5 ml/25 cm² of a solution containing 0.05% trypsin/0.5 mM EDTA. The trypsin/EDTA was evacuated and the wash repeated. After evacuation, the flask was placed incubated at 37° C. for 5 minutes. The cells were suspended in medium containing 5% defined calf serum. The cell suspension was centrifuged at 100×g for ten minutes. The cells were resuspended in Amaxa nucleofector solution for mouse ES cells at a concentration of 2×10⁷/ml. The 5 μg of plasmid was added to the cells and the mixture was placed in an electroporation cuvette. The cuvette was inserted into the Amaxa nucleofector and the cells were nucleofected using program A23. The cells were gently suspended in 500 μl of medium and incubated at 37° C. for 15 minutes. The solution was added to 30 ml of EG medium and 5 ml was distributed into each well of a 6 well plate.

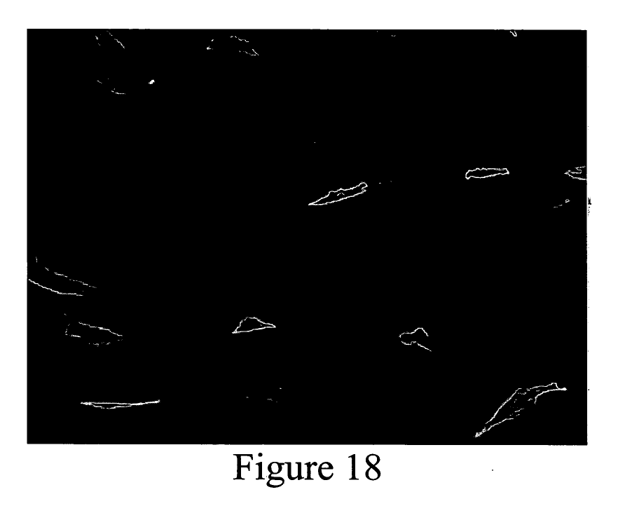

Fluorescence from the GFP plasmid was visible within a few hours of transfection. After 24 hours, the cells were examined and cells that had not attached to the culture dish were counted. Using this procedure, an 80% survival rate was obtained. After counting the cells in a particular field under both phase and fluorescent illumination, a 46% transfection rate was calculated. A typical field is shown in FIG. 18.

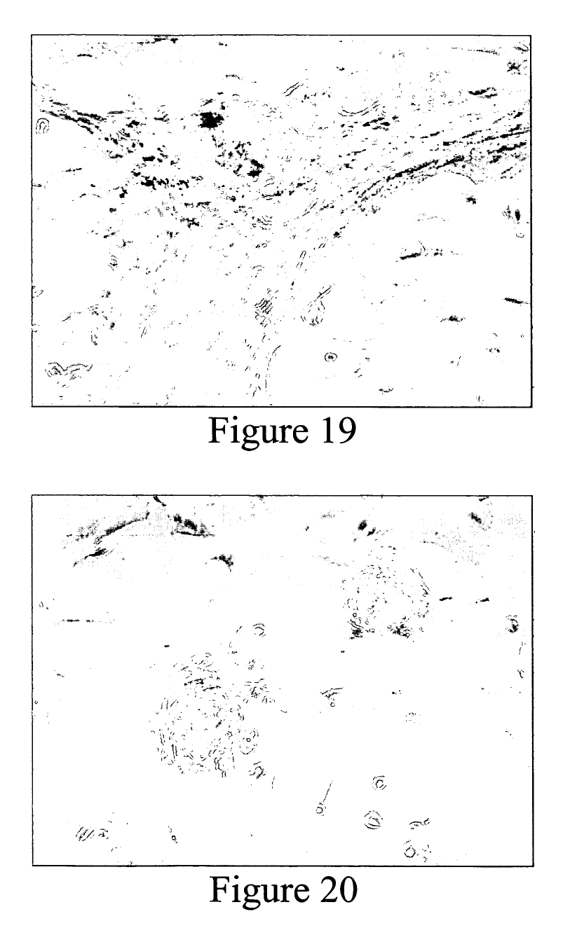

The cells were switched to medium containing 5% defined calf serum (Hyclone) and examined every day for 30 days. The cells continued to proliferate and began to differentiate within one week. A variety of structures formed in situ, as well as hollow bodies resembling embryoid bodies, shown in FIGS. 19 and 20.

### H. REFERENCES

Alberts, B, Bray, D, Lewis, J, Raff, M, Roberts, K, Watson, J D. (1994) MOLECULAR BIOLOGY OF THE CELL, 3^{rd} Ed., Garland Publishing, New York, N.Y., 1294p.
Andrew, P W (2002) From teratocarcinomas to embryonic stem cells. Philos. Trans. R. Soc. Lond. B. Biol. Sci. 357, 405-417.
Asahina, K., Fujimora, H., et al. (2004) Expression of the liver specific gene cyp7A1 reveals hepatic differentiation in embryoid bodies derived from mouse embryonic stem cells. Genes Cells 9, 1297-1308.
Assady S, Maor G, Amit M, Itskovitz-Eldor J, Skorecki K L, Tzukerman M 2001 Insulin production by human embryonic stem cells. Diabetes 50:1691-7
Aubert, J., Stavridis, M. P. (2003) Screening for mammalian neural genes via fluorescence activated cell sorter purification of neural precursors from Sox1-gfp knock-in mice. Proc. Natl. Acad. Sci. 100, Suppl. 1, 11836-11841.
Bain, G., Kitchens, D., Yao, M., Huettner, J. E. and Gottlieb, D. I. (1995) Dev. Biol. 168, 342-357
Barone, M V, Courtneidge, S A. (1995) Myc but not fos rescue of PDGF signaling block by kinase inactive src. Nature. 1995 Nov. 30; 378(6556):509-12
Ben-Hur T, Idelson M, Khaner H, et al. 2004 Transplantation of human embryonic stem cell-derived neural progenitors improves behavioral deficit in Parkinsonian rats. Stem Cells 22:1246-55
Branda, C. S., Dymecki, S. M. (2004) Talking about a revolution: the impact of site specific recombinases on genetic analyses in mice. Developmental Cell 6, 7-28.
Brolen G K, Heins N, Edsbagge J, Semb H 2005 Signals from the embryonic mouse pancreas induce differentiation of human embryonic stem cells into insulin-producing beta-cell-like cells. Diabetes 54:2867-74
Carpenter M K, Inokuma M S, Denham J, Mujtaba T, Chiu C P, Rao M S 2001 Enrichment of neurons and neural precursors from human embryonic stem cells. Exp Neurol 172:383-97
Carpenter, M. K. et al. (2001) Exp. Neurol. 172:383.
Cattaneo, C. et al. (1990) Nature 347:762.
Cerdan C, Rouleau A, Bhatia M 2004 VEGF-A165 augments erythropoietic development from human embryonic stem cells. Blood 103:2504-12
Chadwick K, Wang L, Li L, et al. 2003 Cytokines and BMP-4 promote hematopoietic differentiation of human embryonic stem cells. Blood 102:906-15
Chambers, I., et al. (2003) Functional expression cloning of Nanog, a pluripotency sustaining factor in embryonic stem cells. Cell 113, 643-655
Coraux C, Nawrocki-Raby B, Hinnrasky J, et al. 2005 Embryonic stem cells generate airway epithelial tissue. Am J Respir Cell Mol Biol 32:87-92
Darlington, G J, Ross, S E, MacDougald, O A. (1998) the role of C/EBP genes in adipocyte differentiation. J. Biol. Chem. 273, 30057-30060.
de Miguel, M P, Donovan, P J. (2002) Isolation and culture of embryonic germ cells. Meth. Enzymol. 365, 353-363.
DeClue, J E, Stone, J C, Blanchard, R A, Papageorge, A G, Martin, P, Zhang, K, Lowy, D R. (1991) A ras effector domain mutant which is temperature sensitive for cellular transformation: interactions with GTPase-activating protein and NF-1. Mol. Cell. Biol. 11, 3132-3138.
Denham M, Trounson A, Mollard R. Respiratory Lineage Differentiation of Embryonic Stem Cells In Vitro. Keystone Symposia—Stem Cells, Colorado, USA, 2002
Downward, J. (2002) Targeting RAS signalling pathways in cancer therapy. Nat. Rev. Cancer 3, 11-22.
Downward, J. (2003) Targeting ras signaling pathways in cancer therapy. Nat. Rev. Cancer 3, 11-22.
Dymecki, S (1996) Flp recombinase promotes site specific DNA recombination in embryonic stem cells and transgenic mice. Proc. Natl. Acad. Sci. 93, 6191-6196.
Engelmann, G. L., et al. (1993) Formation of fetal rat cardiac cell clones by retroviral transformation: retention of select myocyte characteristics. J. Mol. Cell. Cardiol. 25, 197-213.
Evans, M J, Kaufman, M H (1981) Establishment in culture of pluripotential cells from mouse embryos. Nature 292, 154-156.
Fahnestock, M L, Lewis, J B. (1989) Limited temperature sensitive transactivation by adenovirus type 2 E1a proteins. J. Virol. 63, 2348-2351.
Fairchild, P J, Nolan, K F, Waldmann, H. (2003) Probing dendritic cell function by guiding the differentiation of embryonic stem cells. Meth. Enzymol. 365, 169-186.
Fiordalisi, J J, Holly, S P, Johnson, R L, Parise, L V, Cox A D. (2002) A distinct class of dominant negative Ras mutants: cytosolic GTP-bound Ras effector domain mutants that inhibit Ras signaling and transformation and enhance cell adhesion. J Biol Chem. 29, 10813-23.
Fraichard, A., Chassande, O., Bilbaut, G., Dehay, C., Savatier, P. and Samarut, J. (1995) J. Cell Sci. 108, 3181-3188
Frederiksen, K. et al. (1988) J. Neurosci. 8:1144.
Frederiksen, K. et al. (1988) Neuron 1:439.
Gearhart, J (1998) New potential for human embryonic stem cells. Science 282, 1061-1062.
Gearhart, J D. (2000) Human embryonic pluripotent germ cells. U.S. Pat. No. 6,090,622.
Gilbert, S F. (1994) DEVELOPMENTAL BIOLOGY, 4^{th} Ed. Sinauer Associates, Inc. Sunderland, M A., p 354.
Green H, Easley K, Iuchi S 2003 Marker succession during the development of keratinocytes from cultured human embryonic stem cells. Proc Natl Acad Sci USA 100:15625-30
Hartley, J. L., Temple, G. F., Brasch, M. A. (2000) DNA cloning using in vitro site-specific recombination. Genome Res. 10, 1788-1795.
Hassink R J, Brutel de la Riviere A, Mummery C L, Doevendans P A 2003 Transplantation of cells for cardiac repair. J Am Coll Cardiol 41:711-7
He J Q, Ma Y, Lee Y, Thomson J A, Kamp T J 2003 Human embryonic stem cells develop into multiple types of cardiac myocytes: action potential characterization. Circ Res 93:32-9
Hematopoietic colony-forming cells derived from human embryonic stem cells. Proc Natl Acad Sci USA 98:10716-21
Hilger, R A, Scheulen, M E, Strumberg, D. (2002) The Ras-raf-mek-erk pathway in the treatment of cancer. Onkologie 25, 511-518.
Hockfield, S. et al. (1985) J. Neurosci. 5:3310.
Hogan, B L M, Costantini, F, Lacy, E. (1986) MANIPULATING THE MOUSE EMBRYO, Cold Spring Harbor Laboratory Press, Cold Spring Harbor, N.Y., 332p.
Hogan, B L M. (1995) Pluripotential embryonic stem cells and methods of making same. U.S. Pat. No. 5,453,357.
Hogan, B L M. (1997) Pluripotential embryonic cells and methods of making same. U.S. Pat. No. 5,690,926.
Hogan, B L M. (1997) Pluripotential embryonic stem cells and methods of making same. U.S. Pat. No. 5,670,372.
Hui, T, Rozga, J, & Demetriou, A A (2001) Bioartificial liver support. J. Hepatobiliary Pancreat Surg. 8, 1-15.
Jat, P S, Noble, M D, Ataliotis, P, Tanaka, Y, Yannoutsos, N, Larsen, L, Kioussis, D. (1991) Direct derivation of conditionally immortal cell lines from an H-2 Kb-tsA58 transgenic mouse. Proc. Natl. Acad. Sci. 88, 5096-5100.
Kaufman D S, Hanson E T, Lewis R L, Auerbach R, Thomson J A 2001
Kawasaki H, Suemori H, Mizuseki K, et al. 2002 Generation of dopaminergic neurons and pigmented epithelia from primate ES cells by stromal cell-derived inducing activity. Proc Natl Acad Sci USA 99:1580-5
Kehat I, Khimovich L, Caspi O, et al. 2004 Electromechanical integration of cardiomyocytes derived from human embryonic stem cells. Nat Biotechnol 22:1282-9
Kelly, J. H., Darlington, G. J. (1985) Hybrid genes: molecular approaches to tissue specific gene regulation. Ann. Rev. Gen. 19, 273-296.
Kelly, J. H., Sussman, N. L. (2000) A fluorescent cell based assay for cytochrome P450 isozyme 1A2 induction and inhibition. J. Biomol. Screen. 5, 249-254.
Kelly, J H, Darlington, G J. (1985) Hybrid genes: molecular approaches to tissue specific gene regulation. Ann. Rev. Gen. 19, 273-296.
Kelly, J H, Spiering, A L, Sussman, N L (1997) Pathogen free human serum protein production using a hollow-fiber bioreactor system. IVD Technology 3, 30-37.
Kelly, J H, Sussman, N L (2000) A fluorescent cell-based assay for cyp1A2 induction and inhibition. J. Biomol. Scr. 5, 249-253.
Kelly, J H. (1994) Permanent human hepatocyte cell line and its use in a liver assist device (LAD). U.S. Pat. No. 5,290,684.
Koshimuzu, U., et al. (1996) Functional requirement of gp 130-mediated signaling for growth and survival of mouse primordial germ cells in vitro and derivation of embryonic germ (EG) cells. Development 122, 1235-1242.
Kubo A, Shinozaki K, Shannon J M, et al. 2004 Development of definitive endoderm from embryonic stem cells in culture. Development 131:1651-62
Kyba, M, Perlingeiro, R C R, Daley, G Q (2003) Development of hematopoietic repopulating cells from embryonic stem cells. Meth. Enzymol. 365, 114-129.
Labosky, P A, Barlow, D P, Hogan B LM (1994) Mouse embryonic germ (EG) cell lines: transmission through the germline and differences in the methylation imprint of insulin-like growth factor 2 receptor (Igfr2) gene compared with embryonic stem (ES) cell lines. Development 120, 3197-3204.
Lavon N, Yanuka O, Benvenisty N 2004 Differentiation and isolation of hepatic-like cells from human embryonic stem cells. Differentiation 72:230-8
Lechner, A. et al. (2002) Biochem. Biophys. Res. Commun. 293:670.
Lee, S. H., Lumelsky, N., Studer, L., Auerbach, J. M. and McKay, R. D. (2000) Nat. Biotechnol. 18, 675-679
Lendahl, U. et al. (1990) Cell 60:585.
Lendahl, U., Zimmerman, L. B. and McKay, R. D. G. (1990) Cell 60, 585-595
Lewandowski, M. (2001) Conditional control of gene expression in the mouse. Nat. Rev. Genet. 2, 743-755.
Li X J, Du Z W, Zarnowska E D, et al. 2005 Specification of motoneurons from human embryonic stem cells. Nat Biotechnol 23:215-21
Li, H., Roblin, G., Liu, H, Heller, S. (2003) Generation of hair cells by stepwise differentiation of embryonic stem cells. Proc. Natl. Acad. Sci. 100, 13495-13500.
Liu, S., Qu, Y., Stewart, T. J., Howard, M. J., Chakrabortty, S., Holekamp, T. F. and McDonald, J. W. (2000) Proc. Natl. Acad. Sci. U.S.A. 97, 6126-6131
Liu, Y. P., et al. (2004) Maintenance of pluripotentcy in human embryonic stem cells stably over-expressing enhanced green fluorescent protein. Stem Cells Dev. 13, 636-645.
Loebel D A, Watson C M, De Young R A, Tam P P 2003 Lineage choice and differentiation in mouse embryos and embryonic stem cells. Dev Biol 264:1-14
Lumelsky N, Blondel O, Laeng P, Velasco I, Ravin R, McKay R 2001 Differentiation of embryonic stem cells to insulin-secreting structures similar to pancreatic islets. Science 292:1389-94
Lumelsky, N. et al. (2001) Science 292:1389.
Macneish, J. (2004) Stem cells in drug discovery. Nat. Rev. Drug Disc. 3, 70-80.
Martin, G R (1981) Isolation of a pluripotent cell line from early mouse embryos cultured in medium conditioned by teratocarcinoma stem cells. Proc. Natl. Acad. Sci. 78, 7634-7638.
Matsui, Y, Zsebo, K, Hogan, BLM (1992) Derivation of pluripotential embryonic stem cells from murine primordial germ cells in culture. Cell 70, 841-847.
Matsui, Y., Zsebo, K., Hogan, B. L. (1992) Derivation of pluripotent embryonic stem cells from murine primordial germ cells in culture. Cell 70, 841-847.
McDonald J W, Becker D, Holekamp T F, et al. 2004 Repair of the injured spinal cord and the potential of embryonic stem cell transplantation. J Neurotrauma 21:383-93
Millis, J M, Cronin D C, Johnson, R, Conjeevarum, H, Conlin, C, Trevino, S, & Maguire, P. (2002) Initial experience with the modified extracorporeal liver assist device for patients with fulminant hepatic failure: system modifications and clinical impact. Transplantation 74, 1735-1746.
Mintz, B, Illmensee, K. (1975) Normal genetically mosaic mice produced from malignant teratocarcinoma cells. Proc. Natl. Acad. Sci. 72, 3585-3589.
Misra, R P, Duncan, S A (2002) Gene targeting in the mouse: advances in introduction of transgenes into the genome by homologous recombination. Endocrine 19, 229-238.
Mitalipova, M. M., et al. (2005) preserving the genetic integrity of human embryonic stem cells. Nat. Biotechnol. 23, 19-20.
Mizuseki K, Sakamoto T, Watanabe K, et al. 2003 Generation of neural crestderived peripheral neurons and floor plate cells from mouse and primate embryonic stem cells. Proc Natl Acad Sci USA 100:5828-33
Mummery C, Ward D, van den Brink C E, et al. 2002 Cardiomyocyte differentiation of mouse and human embryonic stem cells. J Anat 200:233-42
Mummery C, Ward-van Oostwaard D, Doevendans P, et al. 2003 Differentiation of human embryonic stem cells to cardiomyocytes: role of coculture with visceral endoderm-like cells. Circulation 107:2733-40
Muraca, M., Neri, D., et al. (2002) Intraportal hepatocyte transplantation in the pig: hemodynamic and histopathological study. Transplantation 73, 890-896.
Ng E S, Azzola L, Sourris K, Robb L, Stanley E G, Elefanty A G 2005 The primitive streak gene Mixl1 is required for efficient haematopoiesis and BMP4-induced ventral mesoderm patterning in differentiating ES cells. Development 132:873-84
Ng E S, Davis R P, Azzola L, Stanley E G, Elefanty A G 2005 Forced aggregation of defined numbers of human embryonic stem cells into embryoid bodies fosters robust, reproducible hematopoietic differentiation. Blood 106:1601-03
Nistor G I, Totoiu M O, Haque N, Carpenter M K, Keirstead H S 2005 Human embryonic stem cells differentiate into oligodendrocytes in high purity and myelinate after spinal cord transplantation. Glia 49:385-96
Okabe, S., Forsberg-Nilsson, K., Spiro, A. C., Segal, M. and McKay, R. D. (1996) Mech. Dev. 59, 89-102
Organ, E. L., Sheng, J., Ruley, H. E., Rubin, D. H. (2004) Discovery of mammalian genes that participate in virus infection. BMC Cell Biology 5, 41.
Park C H, Minn Y K, Lee J Y, et al. 2005 In vitro and in vivo analyses of human embryonic stem cell-derived dopamine neurons. J Neurochem 92:1265-76
Park S, Lee K S, Lee Y J, et al. 2004 Generation of dopaminergic neurons in vitro from human embryonic stem cells treated with neurotrophic factors. Neurosci Lett 359:99-103
Pera M F, Andrade J, Houssami S, et al. 2004 Regulation of human embryonic stem cell differentiation by BMP-2 and its antagonist noggin. J Cell Sci 117:1269-80
Pera M F, Trounson A O 2004 Human embryonic stem cells: prospects for development. Development 131:5515-25
Pera, M F, Reubinoff, B, and Trounson, A (2000) Human embryonic stem cells. J. Cell Sci. 113, 5-10.
Perrier A L, Tabar V, Barberi T, et al. 2004 Derivation of midbrain dopamine neurons from human embryonic stem cells. Proc Natl Acad Sci USA 101:12543-8
Pettite, J. N., Liu, G., Yang, Z. (2004) Avian pluripotent stem cells. Mech. Dev. 121, 1159-1168.
Pinkert, C A, Omitz, D M, Brinster, R L, Palmiter, R D. (1987) An albumin enhancer located 10 kb upstream functions along with its promoter to direct efficient, liver-specific expression in transgenic mice. Genes Dev. 3, 268-76.
Rajagopal J, Anderson W J, Kume S, Martinez O I, Melton D A 2003 Insulin staining of ES cell progeny from insulin uptake. Science 299:363
Rambhatla L, Chiu C P, Kundu P, Peng Y, Carpenter M K 2003 Generation of hepatocyte-like cells from human embryonic stem cells. Cell Transplant 12:1-11
Rambhatla, L, Chiu, C P, Kundu, P, Peng, Y, Carpenter, M K. (2003) Generation of hepatocyte-like cells from human embryonic stem cells. Cell Transplant. 12, 1-11.
Ray, M. K., Fagan, S. P., Brunicardi, F. c. (2000) The Cre-loxP system: a versatile tool for targeting genes in a cell and stage specific manner. Cell Transplant. 9, 805-815.
Resnick, J. L., Bixler, L. S., Cheng, L., and Donovan, P. J. (1992) Long-term proliferation of mouse primordial germ cells in culture. Nature 359, 550-551.
Reubinoff B E, Itsykson P, Turetsky T, et al. 2001 Neural progenitors from human embryonic stem cells. Nat Biotechnol 19:1134-40
Reubinoff B E, Pera M F, Fong C Y, Trounson A, Bongso A 2000 Embryonic stem cell lines from human blastocysts: somatic differentiation in vitro. Nat Biotechnol 18:399-404
Reynolds, B. A. et al. (1992) Science 255:1707.
Rietze, R. L. et al. (2001) Nature 412:736.
Rodda, D. J., et al. (2005) Transcriptional regulation of nanog by OCT4 and SOX2. J. Biol. Chem. 280, 24731-24737.
Roybon L, Brundin P, Li J Y 2005 Stromal cell-derived inducing activity does not promote dopaminergic differentiation, but enhances differentiation and proliferation of neural stem cell-derived astrocytes. Exp Neurol:Epub ahead of print
Saez, E, Nelson, M C, Eshelman, B, Banayo, E, Koder, A, Cho, G J, Evans, R M. (2000) Identification of ligands and coligands for the ecdysone-regulated gene switch. Proc. Natl. Acad. Sci. 97, 14512-14517.
Sandig, V, Loser, P, Lieber, A, Kay, M A, Strauss, M. (1996) HBV-derived promoters direct liver-specific expression of an adenovirally transduced LDL receptor gene. Gene Therapy 3, 1002-1009.
Sauer. B. (2002) Cre/lox: one more step in the taming of the genome. Endocrine 19, 221-228.
Schaft, J, Ashery-Padan, R, van der Houven, F, Gruss, P, Stewart, A F. (2001) Efficient F LP recombination in mouse ES cells and oocytes. Genesis 31, 6-10.
Schuldiner, M., Itskovitz-Elder, J., Benvenisty, N. (2003) Selective ablation of human embryonic stem cells expressing a “suicide” gene. Stem Cells 21, 257-265.
Schulz T C, Noggle S A, Palmarini G M, et al. 2004 Differentiation of human embryonic stem cells to dopaminergic neurons in serum-free suspension culture. Stem Cells 22:1218-38
Schulz T C, Palmarini G M, Noggle S A, Weiler D A, Mitalipova M M, Condie B G 2003 Directed neuronal differentiation of human embryonic stem cells. BMC Neurosci 4:27
Segev H, Fishman B, Ziskind A, Shulman M, Itskovitz-Eldor J 2004 Differentiation of human embryonic stem cells into insulin-producing clusters. Stem Cells 22:265-74
Shamblott, M. J., Axelman, J., et al. (1998) Derivation of pluripotent stem cells from cultured human primordial germ cells. Proc. Natl. Acad. Sci. 95, 13726-13731.
Shamblott, M J, Axelman, J, Wang, S, Bugg, E M, Littlefield, J W, Donovan, P J, Blumenthal, P D, Huggins, G R, Gearhart, J D. (1998) Derivation of pluripotent stem cells from cultured human primordial germ cells. Proc. Natl, Acad. Sci. 95, 13726-13731.
Shin, S, Dalton, S, and Stice, S L (2005) Human motor neurons differentiation from human embryonic stem cells. Stem Cells and Dev. 14, 266-269.
Shih, C. C. et al. (2001) Blood 98:2412
Stamp L A, Crosby H A, Hawes S M, Strain A J, Pera M F.Characterisation of GCTM5, a putative marker for early liver cells. First National Stem Cell Centre Annual Conference—Stem Cells and Tissue Repair, Melbourne, Australia, 2003, p 28
Steinert, P. M. et al. (1999) J. Biol. Chem. 274:9881.
Sterneckert, J L, Hill, C M, Palmer, R, Gearhart, J D (2005) Bone morphogenetic proteins produced by cells within embryoid bodies inhibit ventral directed differentiation by sonic hedgehog. Clon. Stem Cells 7, 27-34.
Strubing, C., Ahnert-Hilger, G., Shan, J., Wiedemann, B., Hescheler, J. and Wobus, A. M. (1995) Mech. Dev. 53, 275-287
Suchard, J. (2001) Review: wherefore withdrawal, The science behind recent drug withdrawals and warnings. Int. J. Med. Toxicol. 4, 15-20.
Sucharov, C. C., et al. (2004) The Ku protein complex interacts with YY1, is up-regulated in human heart failure, and represses a myosin heavy chain gene expression. Mol. Cell. Biol. 24, 8705-8715.
Sugden, P. H. (2003) Ras, Akt and mechanotransduction in the cardiac myocyte. Circ. Res. 93, 1179-1192.
Sussman, N L & Kelly, J H. (1995) The Artificial Liver. Scientific American: Science and Medicine 2, 68-77.
Sussman, N L, Chong, M G, Koussayer, T, He, D, Shang, T A, Whisennand, H H & Kelly, J H. (1992) Reversal of fulminant hepatic failure using an extracorporeal liver assist device. Hepatology 16, 60-65.
Sussman, N L, Gislason, G T, Conlin, C A, & Kelly, J H. (1994) The Hepatix extracorporeal liver assist device: initial clinical experience. Artificial Organs 18, 390-396.
Sussman, N L, Kelly, J H. (1994) Organ support system. U.S. Pat. No. 5,368,555.
Thomson, J A (1998) Primate embryonic stem cells. U.S. Pat. No. 5,843,780.
Thomson, J A, Itskovitz-Eldor, J, Shapiro, S S, Waknitz, M A, Swiergiel, J J, Marshall, V S, Jones, J M. (1998) Embryonic stem cell lines derived from human blastocysts. Science 282, 1145-1147.
Tohyama, T. et al. (1992) Lab. Invest. 66:303.
Trounson A 2004 Stem cells, plasticity and cancer—uncomfortable bed fellows. Development 131:2763-8
Trounson A 2005 Derivation characteristics and perspectives for mammalian pluripotential stem cells. Reprod Fertil Dev 17:135-41
Trounson, A (2001) Human embryonic stem cells: mother of all cell and tissue types. Reprod Fertil Dev. 2001; 13(7-8):523-32.
Tsung, H. C., et al. (2003) The culture and establishment of embryonic germ (EG) cell lines from Chinese mini swine. Cell Res. 13, 195-202.
Turnpenny, L., Brickwood, S., et al. (2003) Derivation of human embryonic germ cells: an alternative source of pluripotent stem cells. Stem Cells 21, 598-609.
Uchida, N. et al. (2000) Proc. Natl. Acad. Sci. USA 97:14720.
van de Waterbeemd, H, Gifford, E. (2003) ADMET in silico modeling: towards prediction paradise. Nat. Rev. Drug Disc. 2, 192-204.
Wassarman, P M, Keller, G M. (2003) METHODS IN ENZYMOLOGY, Differentiation of Embryonic Stem Cells, vol. 365, Elsevier Academic Press, New York, N.Y., 510p.
Wasserman, P. M., Keller, G. M. (2003) “DIFFERENTIATION OF EMBRYONIC STEM CELLS”. Methods Enzymol., Vol. 235, Elsevier Academic Press, Amsterdam.
Wichterle, H., Lieberam, I., Porter, J. and Jessell, T. (2002) Cell 110, 385
Wiese C, Rolletschek A, Kania G, Blyszczuk P, Tarasov K V, Tarasova Y, Wersto R P, Boheler K R, Wobus A M. Nestin expression—a property of multi-lineage progenitor cells? Cell Mol Life Sci. 2004 October; 61(19-20):2510-22.
Willis A, et al., Mutant p53 exerts a dominant negative effect by preventing wild-type p53 from binding to the promoter of its target genes, Oncogene. 2004 Mar. 25; 23(13):2330-8.
Xu R H, Chen X, Li D S, et al. 2002 BMP4 initiates human embryonic stem cell differentiation to trophoblast. Nat Biotechnol 20:1261-4
Xue T, Cho H C, Akar F G, et al. 2005 Functional integration of electrically active cardiac derivatives from genetically engineered human embryonic stem cells with quiescent recipient ventricular cardiomyocytes: insights into the development of cell based pacemakers. Circulation 111:11-20
Yamauchi-Takihara, K., et al. (1989) Characterization of human cardiac myosin heavy chain genes. Proc. Natl, Acad. Sci. 86, 3504-3508.
Yan Y, Yang D, Zarnowska E D, et al. 2005 Directed differentiation of dopaminergic neuronal subtypes from human embryonic stem cells. Stem Cells 23:781-90
Ye, W., Shimamura, K., Rubenstein, J. L., Hynes, M. A. and Rosenthal, A. (1998) Cell 93, 755-766
Zambrowicz, B P, Sands, AT (2003) Knockouts model the 100-best selling drugs—will they model the next 100? Nat. Rev. Drug Disc. 2, 38-51.
Zeng X, Cai J, Chen J, et al. 2004 Dopaminergic differentiation of human embryonic stem cells. Stem Cells 22:925-40
Zhang S C, Wernig M, Duncan I D, Brustle O, Thomson J A 2001 In vitro differentiation of transplantable neural precursors from human embryonic stem cells. Nat Biotechnol 19:1129-33
Zulewski, H. et al. (2001) Diabetes 50:521.
Zuniga-Pflucker, J. C. (2004) T-cell development made simple. Nat. Rev. Immunol. 4, 67-72.
Zweigerdt, R., Burg, M., Willbold, E., Abts, H. F., Ruediger, M. (2001) Generation of confluent cardiomyocyte monolayers derived from embryonic stem cells in suspension: a cell source for new therapies and screening strategies. Cytotherapy 5, 399-413.

What is claimed is:

1. An isolated stem cell derived from gonadal ridge or testes of fetal or embryonic material that can be maintained without a feeder layer for at least 20 passages, wherein the cell:
(a) is grown in a culture medium that has not been conditioned by a feeder layer,
(b) maintains the potential to differentiate into derivatives of endodermal, mesodermal, and ectodermal cells throughout the culture, and
(c) maintains a normal karyotype.

2. The isolated stem cell of claim 1, wherein the stem cell is derived from a primordial germ cell (PGC).

3. The isolated stem cell of claim 1, wherein the stem cell is a PC.

4. The isolated stem cell of claim 1, wherein the cell stains positive for the SSEA-1 antigen, stains negative for SSEA-4 antigen, and stains positive for alkaline phosphatase.

5. The isolated stem cell of claim 1, wherein the stem cell is directly contacting a solid substrate.

6. The isolated stem cell of claim 1, wherein the stem cell is replated as a single cell suspension.

7. The isolated stem cell of claim 1, wherein the culture medium comprises oncostatin M sufficient to maintain the stem cell without a feeder layer for at least 20 passages.

8. The isolated stem cell of claim 1, wherein the culture medium comprises forskolin sufficient to maintain the stem cell without a feeder layer for at least 20 passages.

9. The isolated stem cell of claim 1, wherein the culture medium comprises FGF sufficient to maintain the stem cell without a feeder layer for at least 20 passages.

10. The isolated stem cell of claim 1, wherein the culture medium comprises stem cell factor (SCF) sufficient to maintain the stem cell without a feeder layer for at least 20 passages.

11. A culture medium for growing stem cells in the absence of a feeder layer, comprising a base medium suitable for growing stem cells, stem cell factor and oncostatin M sufficient to grow stem cells without a feeder layer.

12. The culture medium of claim 11, wherein the culture medium allows the maintenance of the stem cells without a feeder layer for at least 20 passages.

13. The culture medium of claim 11, comprising at least 5 uM of oncostatin M.

14. The culture medium of claim 11, comprising at least 5 uM forskolin.

15. The culture medium of claim 11, comprising at least 5 ng per ml FGF.

16. The culture medium of claim 11, comprising at least 5 ng per ml stem cell factor.

17. A composition comprising the isolated stem cell of claim 1, in the culture medium of claim 10.

18. The composition of claim 17, wherein the stem cell does not contact a feeder layer.

19. A composition comprising the isolated stem cell of claim 1, growing on plastic without a feeder layer.

20. A method of isolating a stem cell, comprising
(a) providing fetal gonadal tissue from an embryo;
(b) culturing said tissue directly on a solid substrate in culture medium comprising a suitable amount of growth factors wherein one of the growth factors is oncostatin M;
(c) selecting cells that exhibit the following characteristics:
(i) maintains a normal karyotype for at least 20 passages and
(ii) maintains the potential to differentiate into derivatives of endodermal, mesodermal, and ectodermal cells throughout the culture.

21. The method of claim 20, wherein the fetal gonadal tissue comprises primordial germ cells.

22. The method of claim 20, wherein one of the growth factors is FGF-2.

23. The method of claim 20, wherein one of the growth factors is stem cell factor.

24. The method of claim 20, wherein one of the growth factors is forskolin.

25. The method of claim 20, wherein the pluripotent stem cell is human.

26. An isolated pluripotent human stem cell derived by the method of claim 20.

27. The stem cell of claim 1, wherein the cell is human.

28. The cell of claim 1, wherein the cell is a clone.

29. A stem cell clone, wherein the cell clone is negative for SSEA-4.

30. The stem cell clone of claim 29, wherein the cell clone is positive for alkaline phosphotase.

31. The stem cell clone of claim 29, wherein the cell clone is positive for SSEA-1.

32. The stem cell clone of claim 29, wherein the cell clone is human.

33. The cell of claim 1, wherein the cell does not comprise Neu5Gc.

34. A method of deriving terminally differentiated cells comprising differentiating the cell of claim 1, using tissue specific reversible immortalization.

35. An isolated stem cell derived from gonadal ridge or testes of fetal or embryonic material that can be maintained without a feeder layer for at least 20 passages, wherein the cell:
(a) is grown in a culture medium that has not been conditioned by a feeder layer,
(b) maintains the potential to differentiate into derivatives of endodermal, mesodermal, and ectodermal cells throughout the culture, and
(c) maintains a normal karyotype.

36. An isolated stem cell that can be maintained without a feeder layer for at least 20 passages, wherein the cell:
(a) maintains the potential to differentiate into derivatives of endodermal, mesodermal, and ectodermal cells throughout the culture,
(b) stains negative for SSEA-4 antigen, and
(c) maintains a normal karyotype.

37. The isolated stem cell of claim 36, wherein the cell stains positive for the SSEA-1 antigen.

38. The isolated stem cell of claim 36, wherein the cell is grown in a culture medium that has not been conditioned by a feeder layer.

39. The isolated stem cell of claim 35 or 36, wherein the cell is a human cell.

40. An isolated pluripotent stem cell which stains negative for the SSEA-4 antigen.

41. The isolated stem cell of claim 40, where in the cell stains positive for the SSEA-1 antigen.

42. The isolated stem cell of claim 40, where in the cell maintains a normal karyotype.

43. The isolated stem cell of claim 40, where in the cell maintains the potential to differentiate into derivatives of endodermal, mesodermal, and ectodermal cells throughout the culture.

44. The isolated stem cell of claim 40, wherein the cell stains positive for alkaline phosphatase.

45. The isolated stem cell of claim 40, wherein the cell is derived from a primordial germ cell.

46. The isolated stem cell of claim 40, wherein the cell stains negative for Neu5Gc.

47. An isolated stem cell comprising the following characteristics:
(a) maintains the potential to differentiate into derivatives of endodermal, mesodermal, and ectodermal cells throughout the culture;
(b) stains negative for SSEA-4 antigen;
(c) stains positive for the SSEA-1 antigen;
(d) stains positive for alkaline phosphatase;
(e) stains positive for Oct-4; and
(f) stains negative for nestin.

48. The isolated stem cell of claim 47, wherein the cell can maintain a normal karyotype in prolonged culture.

49. The isolated stem cell of claim 47, wherein the cell is human.

50. An isolated stem cell comprising the following characteristics:
(a) maintains the potential to differentiate into derivatives of endodermal, mesodermal, and ectodermal cells throughout the culture;
(b) stains negative for SSEA-4 antigen;
(c) stains positive for the SSEA-1 antigen;
(d) stains positive for alkaline phosphatase;
(e) stains positive for Oct-4;
(f) stains negative for nestin; and
(g) can maintain a normal karyotype in prolonged culture.

51. The stem cell of claim 50, wherein the cell is human.

52. A composition comprising the stem cell of claim 1 and at least 5 ng/ml of oncostatin M.

53. A composition comprising:
(a) a stem cell;
(b) at least 5 ng/ml of oncostatin M
(c) at least 5 ng/ml of stem cell factor.

54. The composition of claim 53, wherein the stem cell is human.

55. The composition of claim 53, wherein the cell is:
(a) positive for alkaline phosphatase;
(b) positive for Oct-4;
(c) positive for TRA-1-60;
(d) positive for TRA-1-81;
(e) maintains the potential to differentiate into derivatives of endodermal, mesodermal, and ectodermal cells throughout the culture.

56. A method of isolating a nestin positive stem cell, comprising
(a) providing an SSEA4 negative stem cell;
(b) culturing said cells in medium comprising:
(i) at least 5 ng per ml FGF,
(ii) less than 1 ng per ml oncostatin M and SCF;
(c) selecting cells that stains positive for alkaline phosphatase, SSEA-1, Oct-4, and Nestin; and
(d) isolating said stem cell.

57. A method of isolating a nestin positive stem cell comprising:
(a) culturing an SSEA4 negative stem cell in the substantial absence of Oncostatin;
(b) selecting cells which stain positive for Nestin.

58. The method of claim 57, wherein the cells stain positive for alkaline phosphatase, SSEA-1, and Oct-4.

59. A stem cell produced by the method of claim 57.

60. An isolated stem cell that can be maintained without a feeder layer for at least 20 passages, wherein the cell:
(a) maintains the potential to differentiate into derivatives of endodermal, mesodermal, and ectodermal cells throughout the culture,
(b) stains negative for SSEA-4,
(c) stains positive for alkaline phosphatase, SSEA-1, Oct-4, and Nestin, and
(d) maintains a normal karyotype.

61. The isolated stem cell of claim 60, wherein the cell is grown in a culture medium that has not been conditioned by a cell line or feeder layer.

62. The isolated stem of claim 60, wherein the cell is a human cell.

63. An isolated stem cell, wherein the cell stains positive for alkaline phosphatase, SSEA-1, Oct-4, and Nestin and stains negative for the SSEA-4 antigen.

64. The isolated stem cell of claim 63, where in the cell maintains a normal karyotype.

65. The isolated stem cell of claim 63, wherein the cell maintains the potential to differentiate into derivatives of endodermal, mesodermal, and ectodermal cells throughout the culture.

66. The isolated stem cell of claim 63, wherein the cell is derived from a primordial germ cell.

67. The isolated stem cell of claim 63, wherein the cell is a human cell.

68. A method of producing a homogenous population of neural progenitor cells (NPCs), comprising:
(a) providing the oncostatin-independent stem cell (OISC),
(b) culturing said cells in medium comprising FGF and retinoic acid;
(c) selecting cells that exhibit the following characteristics:
(i) stain positive for Nestin,
(ii) stains negative for alkaline phosphatase and Oct-4; and
(d) isolating said NPCs.

69. A homogenous population of neural progenitor cells (NPCs) produced by the method of claim 68.

70. A method of producing a homogenous population of neural progenitor cells (NPCs), comprising:
(a) providing the isolated stem cell of claim 1,
(b) culturing said cell in medium comprising:
(i) at least 5 ng per ml FGF,
(ii) less than 1 ng per ml oncostatin M and SCF;
(c) selecting a stem cell that stains positive for alkaline phosphatase, SSEA-1, Oct-4, and Nestin;
(d) culturing said stem cell in medium comprising FGF and retinoic acid;
(e) selecting cells that exhibit the following characteristics:
(i) stain positive for Nestin,
(ii) stains negative for alkaline phosphatase and Oct-4; and
(f) isolating said NPCs.

71. A homogenous population of neural progenitor cells (NPCs) produced by the method of claim 70.

72. A method of producing a motor neuron cell, comprising:
(a) providing the NPC of claim 71,
(b) culturing said cell in a medium comprising at least 5 ng per ml retinoic acid and at least 5 ng per ml sonic hedgehog;
(c) selecting motor neurons that stain positive for TUJ1;
(d) isolating said motor neurons.

73. A homogenous population of motor neurons produced by the method of claim 72.

74. A method of producing a homogenous population of muscle progenitor cells (myoblasts), comprising,
(a) providing the oncostatin-independent stem cell of claim 63,
(b) culturing said cells in medium comprising FGF, forskolin and bromo-cyclic AMP;
(c) selecting cells that exhibit the following characteristics:
(i) stain positive for alpha-actinin,
(ii) stains negative for alkaline phosphatase and Oct-4; and
(d) isolating said myoblasts.

75. A homogenous population of muscle progenitor cells (myoblasts) produced by the method of claim 74.

76. A method of producing a homogenous population of muscle progenitor cells (myoblasts), comprising:
(a) providing the isolated stem cell of claim 1,
(b) culturing said cell in medium comprising:
(i) at least 5 ng per ml FGF,
(ii) less than 1 ng per ml oncostatin M and SCF;
(c) selecting a stem cell that stains positive for alkaline phosphatase, SSEA-1, Oct-4, and Nestin;
(d) culturing said stem cell in a medium comprising FGF, forskolin and bromo-cyclic AMP;
(e) selecting cells that exhibit the following characteristics:
(i) stain positive for alpha-actinin,
(ii) stains negative for alkaline phosphatase and Oct-4; and
(f) isolating said myoblasts.

77. A homogenous population of muscle progenitor cells (myoblasts) produced by the method of claim 76.

78. A method of producing a smooth muscle cells, comprising:
(a) providing the myolast of claim 77,
(b) culturing said cell in medium comprising at least 5 ng per ml retinoic acid and at least 5 ng per ml sonic hedgehog;
(c) selecting motor neurons that stain positive for TUJ1;
(d) isolating said motor neurons.

79. A homogenous population of motor neurons produced by the method of claim 78.

80. A composition comprising a pluripotent stem cell growing on a solid substrate in the absence of a feeder layer and conditioned media.

81. The composition of claim 80, wherein the pluripotent stem cell is human.

82. The composition of claim 80, comprising Oncostatin.

83. The composition of claim 80, comprising FGF.

84. The composition of claim 80, comprising stem cell factor.

85. The composition of claim 80, comprising foreskolin.

86. The composition of claim 80, wherein the pluripotent stem cell stains positive for Oct4, Sox2, and nanog.

87. The composition of claim 80, wherein the solid substrate is plastic.

88. The composition of claim 80, wherein the pluripotent stem cell stains positive for SSEA4.

89. A composition comprising:
(a) a pluripotent stem cell growing on a solid substrate in the absence of a feeder layer and conditioned media;
(b) Oncostatin;
(c) FGF; and
(d) wherein the pluripotent stem cell stains positive for Oct4, Sox2, and nanog.

90. The composition of claim 89, wherein the pluripotent stem cell is human.

91. A stem cell comprising the following characteristics:
(a) stains positive for nestin;
(b) stains negative for alkaline phosphotase
(c) stains positive for Oct-4.

92. The stem cell of claim 91, wherein the cell is Nanog positive.

93. The stem cell of claim 91, wherein the cell is Sox2 positive.

94. The stem cell of claim 91, wherein the cell is Tcl1 positive.

95. The stem cell of claim 91, wherein the cell is Tbx3 positive.

96. The stem cell of claim 91, wherein the cell is Cripto positive.

97. The stem cell of claim 91, wherein the cell is Stellar positive.

98. The stem cell of claim 91, wherein the cell is Daz1 positive.

99. The stem cell of claim 91, wherein the cell is SSEA-1 positive.

100. The stem cell of claim 91, wherein the cell is SSEA-4 negative.

101. The stem cell of claim 91, wherein the cell is a human cell.

102. An isolated pluripotent stem cell derived from gonadal ridge or testes of fetal or embryonic material that can be maintained without a feeder layer for at least 20 passages, wherein the cell:
(a) is grown in a culture medium that has not been conditioned by a feeder layer,
(b) maintains the potential to differentiate into derivatives of endodermal, mesodermal, and ectodermal cells throughout the culture, and
(c) maintains a normal karyotype.

103. A culture medium for growing pluripotent stem cells in the absence of a feeder layer, comprising a base medium suitable for growing stem cells, stem cell factor and oncostatin M sufficient to grow pluripotent stem cells without a feeder layer.

104. A method of isolating a pluripotent stem cell, comprising
(a) providing fetal gonadal tissue from an embryo;
(b) culturing said tissue directly on a solid substrate in culture medium comprising a suitable amount of growth factors wherein one of the growth factors is oncostatin M;
(c) selecting cells that exhibit the following characteristics:
(i) maintains a normal karyotype for at least 20 passages and
(ii) maintains the potential to differentiate into derivatives of endodermal, mesodermal, and ectodermal cells throughout the culture.

105. A method of deriving terminally differentiated cells comprising differentiating the cell of claim 1, using tissue specific reversible immortalization.

106. An isolated pluripotent stem cell derived from gonadal ridge or testes of fetal or embryonic material that can be maintained without a feeder layer for at least 20 passages, wherein the cell:
(a) is grown in a culture medium that has not been conditioned by a feeder layer,
(b) maintains the potential to differentiate into derivatives of endodermal, mesodermal, and ectodermal cells throughout the culture, and
(c) maintains a normal karyotype.

107. An isolated pluripotent stem cell that can be maintained without a feeder layer for at least 20 passages, wherein the cell:
(a) maintains the potential to differentiate into derivatives of endodermal, mesodermal, and ectodermal cells throughout the culture,
(b) stains negative for SSEA-4 antigen, and
(c) maintains a normal karyotype.

108. An isolated pluripotent stem cell comprising the following characteristics:
(a) maintains the potential to differentiate into derivatives of endodermal, mesodermal, and ectodermal cells throughout the culture;
(b) stains negative for SSEA-4 antigen;
(c) stains positive for the SSEA-1 antigen;
(d) stains positive for alkaline phosphatase;
(e) stains positive for Oct-4; and
(f) stains negative for nestin

109. An isolated pluripotent stem cell comprising the following characteristics:
(a) maintains the potential to differentiate into derivatives of endodermal, mesodermal, and ectodermal cells throughout the culture;
(b) stains negative for SSEA-4 antigen;
(c) stains positive for the SSEA-1 antigen;
(d) stains positive for alkaline phosphatase;
(e) stains positive for Oct-4;
(f) stains negative for nestin; and
(g) can maintain a normal karyotype in prolonged culture.

110. A composition comprising:
(a) a pluripotent stem cell;
(b) at least 5 ng/ml of oncostatin M
(c) at least 5 ng/ml of stem cell factor.

111. An isolated pluripotent stem cell derived from gonadal ridge or testes of fetal or embryonic material that can be maintained without a feeder layer for at least 20 passages, wherein the cell:
(a) is grown in a culture medium that has not been conditioned by a feeder layer,
(b) maintains the potential to differentiate into derivatives of endodermal, mesodermal, and ectodermal cells throughout the culture, and
(c) maintains a normal karyotype.
